(19) United States
(12) Patent Application Publication
(10) Pub. No.: US 2017/0143022 A1
(43) Pub. Date: May 25, 2017

(54) Compositions Incorporating an Umami Flavor Agent

(71) Applicant: Senomyx, Inc., San Diego, CA (US)

(72) Inventors: Sharon Wicker, Carlsbad, CA (US); Tanya Ditschun, San Diego, CA (US)

(21) Appl. No.: 14/948,101

(22) Filed: Nov. 20, 2015

(51) Int. Cl. A01N 25/00; A23K 20/10; A61K 47/22; A23L 2/56; A23K 50/40

(52) U.S. Cl. CPC A23L 27/20; A23L 27/88; A23L 2/56; A23L 27/80; A23L 27/30; A23K 20/10; A23K 50/40; A61K 47/22

(57) ABSTRACT

The present invention relates to compositions containing flavor or taste modifiers, such as a flavoring or flavoring agents and flavor or taste enhancers, more particularly, savory (“umami”) taste modifiers, savory flavoring agents and savory flavor enhancers, for foods, beverages, and other comestible compositions. Compositions comprising an umami flavor agent or umami taste-enhancing agent in combination with one or more other food additives, preferably including a flavorant, herb, spice, fat, or oil, are disclosed.

### BACKGROUND OF THE INVENTION

Field of the Invention

The present invention relates to compositions containing flavor or taste modifiers, such as a flavoring or flavoring agents and flavor or taste enhancers, more particularly, savory (“umami”) taste modifiers, savory flavoring agents and savory flavor enhancers, for foods, beverages, dairy and other comestible compositions.

Description of the Related Art

For centuries, various natural and unnatural compositions and/or compounds have been added to foods, beverages, and/or comestible (edible) compositions to improve their taste. Although it has long been known that there are only a few basic types of “tastes” (sweet, sour, bitter, salty, and “umami”/savory), the biological and biochemical basis of taste perception was poorly understood, and most taste improving or taste modifying agents have been discovered largely by simple trial and error processes.

For example, one of the five known basic tastes is the “savory” or “umami” flavor of monosodium glutamate (“MSG”), which is now commonly added to many food and beverage compositions to desirably improve their “savory” flavor. MSG is known to produce adverse reactions in some people, but very little progress has been made in identifying artificial substitutes for MSG. It is known that a few naturally occurring materials can increase or enhance the effectiveness of MSG as a savory flavoring agent, so that less MSG is needed for a given flavoring application. For example the naturally occurring nucleotide compounds inosine monophosphate (IMP) or guanosine monophosphate (GMP) are known to have a synergistic and/or multiplier effect on the savory taste of MSG. Nevertheless, IMP and GMP are difficult and expensive to isolate and purify from natural sources, or synthesize, and hence have limited practical application to many commercial needs in food compositions. Less expensive compounds that would provide and/or replace the flavor of MSG itself, or multiply the effectiveness of any MSG that is present so as to replace the need for the addition of IMP or GMP additives could be of very high value, especially if the compounds could be used at extremely low concentrations, so as to minimize costs and any possible health risks. It is also desirable to expand the palette of available flavor compounds, creating new flavors, textures, and sensations by combining novel flavorants and flavor enhancers, including umami flavorants and flavor enhancers, with existing or contemplated flavorants or flavor enhancers.

In recent years substantial progress has been made in biotechnology in general, and in better understanding the underlying biological and biochemical phenomena of taste perception. For example, taste receptor proteins have been recently identified in mammals which are involved in taste perception. Particularly, two different families of G protein coupled receptors believed to be involved in taste perception, T2Rs and Ti1Rs, have been identified. (See, e.g., Nelson, et al., Cell (2001) 106(3):381-390; Adler, et al., Cell (2000) 100(6):693-702; Chandrashekar, et al., Cell (2000) 100:703-711; Matsunami, et al., Number (2000) 404:601-604; Li, et al., Proc. Natl. Acad. Sci. USA (2002) 99:4962-4966; Montmayeur, et al., Nature Neuroscience (2001) 4(S):492-498: U.S. Pat. No. 6,462,148; and PCT publications WO 02/06254, WO 00/63166 art, WO 02/064631, and WO 03/001876, and U.S. Patent publication US 2003-0232407 A1. The entire disclosures of the articles, patent applications, and issued patents cited immediately above are hereby incorporated herein by reference, for all purposes, including their disclosures of the identities and structures of T2Rs and T 1Rs mammalian taste receptor proteins and methods for artificially expressing those receptors in cell lines and using the resulting cell lines for screening compounds as potential “savory” flavoring agents.

Whereas the T2R family includes a family of over 25 genes that are involved in bitter taste perception, the T1Rs only includes three members, T1R1, T1R2 and T1R3. (See Li, et al., Proc. Natl. Acad. Sci. USA (2002) 99:4962-4966.) Recently it was disclosed in WO 02/064631 and/or WO 03/001876 that certain T1R members, when co-expressed in suitable mammalian cell lines, assemble to form functional taste receptors. Particularly it was found that co-expression of T1R1 and T1R3 in a suitable host cell results in a functional T1M1/T1RA3 savory (“umami”) taste receptor that responds to savory taste stimuli, including monosodium glutamate. (See Li; et al. (ld.). The references cited above also disclosed assays and/or high throughput screens that measure T1R1/T1R3 or T1R2/T1R3 receptor activity by fluorometric imaging in the presence of the target compounds. It is also known that there is significant interaction between taste receptors allowing umami taste compounds to interact with receptors for other tastes, leading in some cases to synergistic taste effects (see, for example, Shim, et al. (2015) PLoS ONE 10(4): e0124030. doi: 10.1371/journal.pone.0124030) and Kim, et al. (2015), Biochem. Biophys. Res. Commun. 456(2):586-90)

Certain U.S. and international patent applications have been filed by that disclosed the use of certain compounds as umami and/or sweet tastants, and/or synergistic enhancers of the “Umami” taste of MSG, and/or the sweet taste of a variety of natural and artificial sweeteners. See, for example, U.S. utility patent application Ser. No. 10/913,303, filed Aug. 6, 2004 and published as U.S. Patent Publication No. US-2005-0084506-A1 on Apr. 21, 2005; U.S. utility patent application Ser. No. 12/257,017, filed Oct. 23, 2008 and published as U.S. Patent Publication No. US-2009-0111834-A1 on Apr. 30, 2009; U.S. utility patent application Ser. No. 13/336,272, filed Dec. 23, 2011 and published as U.S. Patent Publication No. US-2012-0201763-A1 on Aug. 9, 2012; U.S. utility patent application Ser. No. 14/509,761, filed Oct. 8, 2014 and published as U.S. Patent Publication No. US-2015-0093339-A1 on Apr. 2, 2015; U.S. utility patent application Ser. No. 11/349,071, filed Feb. 6, 2006 and published as U.S. Patent Publication No. US-2006-0263411-A1 on Nov. 23, 2006; U.S. utility patent application Ser. No. 13/406,002, filed Feb. 27, 2012 and published as U.S. Patent Publication No. US-2012-0226047-A1 on Sep. 6, 2012; U.S. utility patent application Ser. No. 12/396,917, filed Mar. 3, 2009 and published as U.S. Patent Publication No. US-2009-0220662-A 1 on Sep. 3, 2009; and PCT Patent Application Serial No. PCT/US04/25419 filed Aug. 6; 2004 and published as PCT Publication WO 2005/041684 on May 12, 2005, and PCT Publication WO 2005/015158 published on Feb. 17, 2005. The entire disclosures of the patent applications cited above are hereby incorporated by reference, for all purposes, including their disclosures of the identities and structures of amide compounds that can serve as potential “savory” or sweet flavoring agents or enhancers. Nevertheless, there is a continuing need for new and improved taste enhancing compositions. Many of these compounds can be arrived at through the combination of umami flavoring agents or umami flavor enhancers with known food additives, including flavorants, texturants, and flavor enhancers.

### SUMMARY OF THE INVENTION

The invention has many aspects, all of which relate to certain non-naturally occurring compounds comprising the group of compounds disclosed in U.S. Patent Application Publications No. U.S. 2005/0084506A1, U.S. 2009/0111834A1, U.S. 2012/0201763A1, U.S. 2015/0093339A1, U.S. 2006/0263411A1, U.S. 2012/0226047A1, and U.S. 2009/0220662A1, incorporated by reference herein. These compounds are known to bind to and activate the T1R1 and T1R3 umami receptors. (See U.S. Patent Application Publications No. U.S. 2008/0306093 and U.S.2009/0220662). In some embodiments the umami flavoring compound or umami flavor enhancer has the formula:

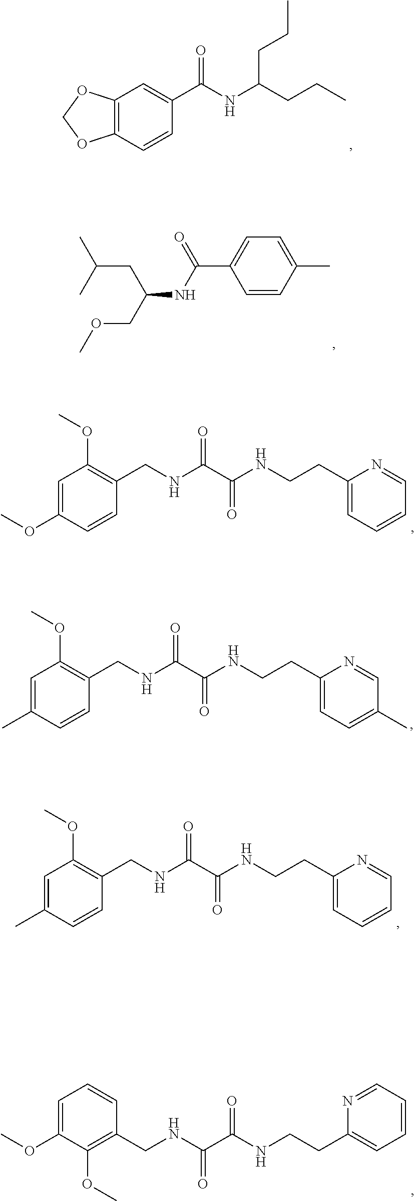

or salts thereof. These compounds, and their method of preparation, are described in the aforementioned published patent applications.

In some embodiments of the invention, the umami flavoring agent or umami flavor enhancer is combined in a suitable amount with a food additive. In other embodiments, the umami flavoring agent or umami flavor enhancer is combined with a flavorant. In other embodiments, the umami flavoring agent or umami flavor enhancer is combined with a sweetener, sweet flavorant, or modulator of sweet taste. In other embodiments, the umami flavoring agent or umami flavor enhancer is combined with a bitterant, bitter flavorant, or modulator of bitter taste. In other embodiments, the umami flavoring agent or umami flavor enhancer is combined with an acid, sour flavorant, or modulator of sour taste. In other embodiments, the umami flavoring agent or umami flavor enhancer is combined with a salt, salty flavorant, or modulator of salty taste. In other embodiments, the umami flavoring agent or umami flavor enhancer is combined with another umami flavoring agent or umami flavor enhancer. In other embodiments, the umami flavoring agent or umami flavor enhancer is combined with an herb or spice. In other embodiments, the umami flavoring agent or umami flavor enhancer is combined with a texturant or modifier of texture or mouthfeel. In other embodiments, the umami flavoring agent or umami flavor enhancer is combined with a fat, oil, essential oil, oleoresin, emulsifier, or emulsion. In other embodiments, the umami flavoring agent or umami flavor enhancer is combined with a savory flavor. In other embodiments the combination of umami flavoring agent or umami flavor enhancer with another food additive, flavorant, flavor enhancer, texturant, fat, oil, emulsion, carrier, or excipient is incorporated into a foodstuff. In other embodiments the combination of umami flavoring agent or umami flavor enhancer with another food additive, flavorant, flavor enhancer, texturant, fat, oil, emulsion, emulsifier, carrier, or excipient is incorporated into a medicament. In other embodiments the combination of umami flavoring agent or umami flavor enhancer with another food additive, flavorant, flavor enhancer, texturant, fat, oil, emulsion, emulsifirer, carrier, or excipient is incorporated into an oral health product. In other embodiments the combination of umami flavoring agent or umami flavor enhancer with another food additive, flavorant, flavor enhancer, texturant, fat, oil, emulsion, carrier, or excipient is incorporated into a confection or candy. In some embodiments the combination of umami flavoring agent or umami flavor enhancer with another food additive, flavorant, flavor enhancer, texturant, fat, oil, emulsion, carrier, or excipient is incorporated into an animal food or animal health product.

### DETAILED DESCRIPTION

Embodiments disclosed herein relate generally to compositions having one or more umami flavor agent or umami flavor enhancer in combination with one or more food additive. In some embodiments, the composition comprises the combination of one or more flavor agents or umami flavor enhancers and one or more flavor modifying compound. The compositions described herein can modify or improve the tastes of flavoring agents, sweeteners, bittering agents, acid/sour flavor agents, or salts or salty flavor agents by imparting an umami taste or a kokumi flavor enhancement. In some embodiments, the flavor modifying compound is one that can activate any taste receptor in vitro and impart an umami or kokumi taste enhancement. In some embodiments, the ingestible compositions provide a more savory flavor profile or a more umami taste. The composition can be further incorporated into a variety of ingestible or non-ingestible compositions. In some embodiments, the ingestible composition comprises one or more flavor modifying compound and one or more umami flavor agents or umami flavor enhancing agents.

In some embodiments, the ingestible composition can be a food or beverage product, or can be incorporated into a food or beverage product.

In some embodiments, the food product can be selected from dried processed foods, such as instant noodles, dehydrated potatoes, dry bread mixes, stuffings, dressings, and the like; chilled processed foods, such as smoked sausages, cold cuts, meat analogues, meat substitutes, pickles, and the like; dairy products, such as milk products, yogurt, sour cream, cheeses, and the like; spreads, such as peanut butter, jams, jellies, mayonnaise and the like; snack foods, such as chips, crisps, crackers, pretzels, nuts and the like; sauces, dressings and condiments, such as ketchup, soy sauce, fish sauce, mustard, salad dressing, and the like; confections, such as cookies, cakes pies, candies, and the like; frozen processed foods such as frozen prepared meats, frozen meat analogues, frozen meat substitutes, vegetables and seafood, frozen pizzas or other prepared dishes, and the like; canned and preserved foods, such as canned vegetables, canned meats, antiseptic-packaged meats, antiseptic-packaged vegetables, and the like; pasta and noodles, including dried noodles, fresh noodles, and pre-prepared noodle mixes and the like; rice and rice mixes and the like, ready meals, including pre-prepared frozen dinners, frozen waffles, frozen sandwiches, and the like; meal replacements, including protein shakes, protein bars, nutritive dietary supplements, and the like; ice cream and frozen treats, including ice cream, sherbet, sorbet, ice cream bars, ice cream sandwiches, frozen custard, gelato, and the like; baked goods, such as breads, rolls, bagels, cakes, muffins, and the like; and baby foods, including prepared vegetables and meats rendered in a soft consistency for consumption by infants and very young children.

In some embodiments, the beverage can be selected from broths, cocktails, including, for example, Bloody Mary, alcoholic beverages, including, for example, beer, malt beverages, hard cider, wine, and distilled spirits, enhanced sparkling beverages, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea-type drinks, coffees, cocoa drinks, beverages containing milk components, beverages containing cereal extracts, soy and soy-based drinks, soy milk, soy milk-based drinks, nut milk and nut-based drinks, including almond and cashew-based drinks and smoothies.

Some embodiments contemplate an ingestible composition, which can be a pharmaceutical product, nutritional product, dietary supplement, or over-the-counter medication. Other embodiments contemplate a non-ingestible composition, which can be an oral care product, hygienic, or cosmetic product.

These and other embodiments, advantages, and features of the present invention are provided in part in the description that follows, and in part will be obvious from the description, or may be learned by practice of the invention. It is to be understood that both the foregoing general description and the following detailed description are exemplary and explanatory only and are not restrictive of the invention as described.

### Definitions:

A “comestibly acceptable carrier or excipient” is a solid or liquid medium and/or composition that is used to prepare a desired dispersed dosage form of the inventive compound, in order to administer the inventive compound in a dispersed/diluted form, so that the biological effectiveness of the inventive compound is maximized. Comestibly acceptable carriers includes many common food ingredients, such as water at neutral, acidic, or basic pH, fruit or vegetable juices, vinegar, marinades, beer, wine, natural water/fat emulsions such as milk or condensed milk, edible oils and shortenings, fatty acids and their alkyl esters, low molecular weight oligomers of propylene glycol, glyceryl esters of fatty acids, and dispersions or emulsions of such hydrophobic substances in aqueous media, salts such as sodium chloride, wheat flours, solvents such as ethanol, solid edible diluents such as vegetable powders or flours, or other liquid vehicles; dispersion or suspension aids; surface active agents; isotonic agents; thickening or emulsifying agents, preservatives; solid binders; lubricants and the like.

A “flavor” herein refers to the perception of taste and/or smell in a subject, which include sweet, sour, salty, bitter, umami, and others. The subject may be a human or an animal.

A “flavoring agent” herein refers to a compound or a biologically acceptable salt thereof that induces a flavor or taste in an animal or a human.

A “flavor modifier” herein refers to a compound or biologically acceptable salt thereof that modulates, including enhancing or potentiating, and inducing, the tastes and/or smell of a natural or synthetic flavoring agent in an animal or a human.

A “flavor enhancer” herein refers to a compound or biologically acceptable salt thereof that enhances and/or multiplies the tastes or smell of a natural or synthetic flavoring agent, or a comestible composition comprising the flavor enhancer.

“Savory flavor” herein refers to the savory “umami” taste typically induced by MSG (monosodium glutamate) in an animal or a human.

“Umami flavoring agent,” “umami compound” or “umami receptor activating compound” herein refers to a compound or biologically acceptable salt thereof that elicits a detectable savory flavor in a subject, e.g., MSG (monosodium glutamate) or a compound that activates a T1R1/T1R3 receptor in vitro. The subject may be a human or an animal.

An “umami flavor modifier” herein refers to a compound or biologically acceptable salt thereof that modulates, including enhancing or potentiating, inducing, and blocking, the savory taste of a natural or synthetic savory flavoring agents, e.g., monosodium glutamate (MSG) in an animal or a human.

An “umami flavor enhancer” herein refers to a compound or biologically acceptable salt thereof that enhances or potentiates the savory taste of a natural or synthetic savory flavoring agents, e.g., monosodium glutamate (MSG) in an animal or a human.

An “umami flavor modulating amount” herein refers to an amount of an umami flavoring agent, umami compound or“umami receptor activating compound that is sufficient to either increase or decrease savory taste in a comestible or medicinal product or composition, or a precursor thereof, sufficiently to be perceived by a human subject. In many, but not all, embodiments of the invention, at least about 0.001 ppm of the compound would need to be present in order for most human subjects to perceive a modulation of the savory flavor of a comestible composition comprising the heterocyclic compound. A broad range of concentration that would typically be employed in order to economically provide a desirable degree of savory flavor modulation can be from about 0.001 ppm to 100 ppm, or a narrow range from about 0.1 ppm to about 10 ppm. Alternative ranges of savory flavor modulating amounts can be from about 0.01 ppm to about 30 ppm, from about 0.05 ppm to about 15 ppm, from about 0.1 ppm to about 5 ppm, or from about 0.1 ppm to about 3 ppm.

An “umami flavor enhancing amount” herein refers to an amount of a compound that is sufficient to enhance the taste of a natural or synthetic flavoring agents, e,g., monosodium glutamate (MSG), disodium inosinate, or disodium guanylate, in a comestible or medicinal product or composition, as perceived by an animal or a human. A broad range of a savory flavor enhancing amount can be from about 0.001 ppm to 100 ppm, or a narrow range from about 0.1 ppm to about 10 ppm. Alternative ranges of savory flavor enhancing amounts can be from about 0.01 ppm to about 30 ppm, from about 0.05 ppm to about 15 ppm, from about 0.1 ppm to about 5 ppm, or from about 0,1 ppm to about 3 ppm.

A “kokumi flavor enhancer” is a compound that produces the kokumi flavor effect, characterized by intensification of flavor experiences generally as described, for example, in Ohsu et at, J. Biol. Chem. 285:1016-1022 (2010).

Umami flavor agents or umami-enhancing agents increase the perceived savory flavor in a taste test. In some embodiments, it is desirable, therefore, that the compositions contemplated herein serve as a replacement or enhancer for MSG, which is not well tolerated by some in, for example, comestible products.

The agonist effect of umami flavor compounds on the T1R1/T1R3 receptor is responsible for the synergistic savory taste effect, which occurs when an umami flavor compound or umami enhancing compound is combined with another savory flavoring agent such as MSG. For example, the nucleotides, inosine monophosphate (IMP) or guanosine monophosphate (GMP), are conventionally added to MSG, to intensify the savory flavor of MSG, so that relatively less MSG is needed to provide the same savory flavor in comparison to MSG alone. Therefore, in some embodiments, combining umami flavor compounds or umami enhancing compounds with another savory flavoring agents such as MSG or the agents disclosed below advantageously eliminates the need to add expensive nucleotides, such as IMP, as a flavor enhancer, while concomitantly reducing or eliminating the amount of a savory compound such as MSG needed to provide the same savory flavor in comparison to the savory compound or MSG alone.

One or more of the compounds that comprise the disclosed compositions may be present as a salt. The term “salt” encompasses those salts that form with the carboxylate anions and amine nitrogens and include salts formed with the organic and inorganic anions and cations discussed below. Furthermore, the term includes salts that form by standard acid-base reactions with basic groups (such as nitrogen containing heterocycles or amino groups) and organic or inorganic acids. Such acids include hydrochloric, hydrofluoric, trifluoroacctic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic and like acids.

The term “organic or inorganic cation” refers to positively charged counter-ions for the carboxylate anion of a carboxylate salt. Inorganic positively charged counter-ions include but are not limited to the alkali and alkaline earth metals, (such as lithium, sodium, potassium, calcium, magnesium, etc.) and other divalent and trivalent metallic cations such as barium, aluminum and the like, and ammonium (NH₄)⁺ cations. Organic cations include ammonium cations derived from acid treatment or alkylation of primary-, secondary, or tertiary amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge, et al., J Pharm. Sci. (1977) 66:1-19, which is incorporated herein by reference. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group is referred to by this term. A preferred cation for the carboxylate anion is the sodium cation.

The compounds that comprise the disclosed compositions can also exist as solvates and hydrates. Thus, these compounds may crystallize with, for example, waters of hydration, or one, a number of, or any fraction thereof of molecules of the mother liquor solvent. Compositions comprising the solvates and hydrates of such compounds are included within the scope of this invention.

The term “amino acid” includes any one of the twenty naturally-occurring amino acids or the D-form of any one of the naturally-occurring amino acids. In addition, the term “amino acid” also includes other non-naturally occurring amino acids besides the D-amino acids, which are functional equivalents of the naturally-occurring amino acids. Such non-naturally-occurring amino acids include, for example, norleucine (“Nle”), norvaline (“Nva”), L- or D-naphthalanine, ornithine (“Orn”), homoarginine (homoArg) and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference. Amino acids and amino acid analogs can be purchased commercially (Sigma Chemical Co.; Advanced Chemtech) or synthesized using methods known in the art.

By the term “effective amount” of a compound as provided herein is meant a sufficient amount of the compound to provide the desired regulation of a desired function, such as gene expression, protein function, or the induction of a particular type of taste perception. As will be pointed out below, the exact amount required will vary from subject to subject, depending on the species, age, general condition of the subject, specific identity and formulation of the comestible composition, etc. Thus, for any given compound or composition it is not possible to specify an exact “effective amount.” However, an appropriate effective amount can be determined by one of ordinary skill in the art using only routine experimentation.

It must be noted that, as used in the specification and the appended claims, the singular forms “a,” “an” and “the” include plural referents unless the context clearly dictates otherwise. Thus, for example, reference to “an aromatic compound” includes mixtures of aromatic compounds.

Often, ranges are expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally including a salt” means that a salt may or may not be included and that the description includes both salt-containing and salt-free compositions.

In some embodiments, the flavor modifying compound is a flavor modifying compound as disclosed in U.S. Application Publication No. 2008/0306093, entitled “Modulation of Chemosensory Receptors and Ligands Associated Therewith”, filed Jun. 8, 2007, which is incorporated herein by reference in its entirety. In some embodiments, the flavor modifying compound is an embodiment of a flavor modifying compound as disclosed in U.S. Application Publication No. 2005/0084506, entitled, “Novel flavors, flavor modifiers, tastants, taste enhancers, umami or sweet tastants, and/or enhancers and use thereof,” filed Aug. 6, 2004, which is incorporated herein by reference in its entirety. In other embodiments, the flavor modifying compound is an embodiment of a flavor modifying compound as disclosed in U.S. Application Publication No. 2006/0263411, entitled “Compounds comprising linked heteroaryl moieties and their use as novel umami flavor modifiers, tastants, and taste enhancers for comestible compositions,” filed. Feb. 6, 2006, which is incorporated herein by reference in its entirety. In other embodiments, the flavor modifying compound is an embodiment of a flavor modifying compound as disclosed in U.S. Application Publication No. 2009/0220662., entitled “Isosorbide derivatives and their use as flavor modifiers, tastants, and taste enhancers,” filed Mar. 3, 2009, which is incorporated herein by reference in its entirety.

### Food Additives

In some embodiments, the composition comprises an umami flavor compound or umami flavor enhancing compound and one or more food additives. Representative food additives include but are not limited to, those declared to the U.S. Food and Drug Administration (See U.S. FDA list of Everything Added to Food in the U.S. (EAFUS), available at http://www.accessdata.fda.gov/scripts/fcn/fcnNavigation.cfm?rpt=eafusListing, last accessed Nov. 16, 2015). Such food additives include: Gum Acacia (Acacia Senegal (L.) Willd.), Acai Berry Extract, Acesulfame Potassium, Acetal, Acetaldehyde, Acetaldehyde, Butyl Phenethyl Acetal, Acetaldehyde Di-Cis-3-Hexenyl Acetal, Acetaldehyde Diisoamyl Acetal, Acetaldehyde Di-Isobutylacetal, Acetaldehyde Ethyl Cis-3-Hexenyl Acetal, Acetaldehyde Ethyl Isobutyl Acetal, (+/−)-Acetaldehyde Ethyl Isopropyl Acetal, Acetaldehyde Hexyl Isoamyl Acetal, Acetaldehyde 1,3-Octanediol Acetal, Acetaldehyde Phenethyl Propyl Acetal, Acetamide, Acetanisole, Acetic Acid, Acetic Anhydride, Acetoin, Acetoin Propyleneglycol Ketal, Alpha-Acetolactate Decarboxylase Enzyme Preparation From Bacillus Subtilis Recombinant, Acetolein, Acetone, Acetone Peroxides, Acetophenone, Acetostearin, 6-Acetoxydihydrotheaspirane, 4-Acetoxy-2,5-Dimethyl-3(2h)-Furanone, (+/−)-1-Acetoxy-1-Ethoxyethane, 4-(P-Acetoxyphenyl)-2-Butanone, 5-Acetyl-2,3-Dihydro-1,4-Thiazine, 2-Acetyl-3,5-Dimethylfuran, 3-Acetyl-2,5-Dimethylfuran, 4-Acetyl-2,5-Dimethyl-3(2h)-Furanone, 2-Acetyl-3,5(Or 6)-Dimethylpyrazine, Mixture Of Isomers, 2-Acetyl-3-Ethylpyrazine, 4-Acetyl-2-Isopropenylpyridine, 2-Acetyl-4-Isopropenylpyridine, 2-Acetyl-4-Isopropylpyridine, 3-Acetylmercaptohexyl Acetate, N-Acetyl-L-Methionine, Acetyl Methyl Carbinyl Acetate, 2-Acetyl-5-Methylfuran, 2-Acetyl-3-Methylpyrazine, 4-Acetyl-2-Methylpyrimidine, 2-Acetyl-5-Methylthiophene, Acetylpyrazine, 2-Acetylpyridine, 3-Acetylpyridine, 2-Acetyl-1-Pyrroline, 4-Acetyl-6-Tert-Butyl-1,1-Dimethylindane, 2-Acetylthiazole, 2-Acetyl-2-Thiazoline, 3-(Acetylthio)-2-Methylfuran, Aconitic Acid, Acrolein, 3-(2-Furyl)Acrolein, Acrylamide-Acrylic Acid Resin, Acrylamide-Sodium Acrylate Resin, Acrylic Acid-2-Acrylamido-2-Methyl Propane Sulfonic Acid Copolymer, Activated Carbon, Adipic Acid, Adipic Anhydride, Advantame, Agar (Gelidium Spp.), Alpha-Bisabolol, Dl-Alanine, L-Alanine, Beta-Alanine; L-Alanyl-L-Glutamine, Albumin, Alcohol, Denatured Formula 23a, Alcohol Sda-3a, Alfalfa, Extract (Medicago Sativa L.), Alfalfa, Herb And Seed (Medicago Sativa L), Algae, Brown, Extract (Macrocystis And Laminaria Spp.), Algae, Red (Porphyra Spp. And Glolopeltis Furcata And Rhodymenia Palmata (L)), Algae, Red, Extract (Porphyra Spp. And Gloiopeltis Furcata And Rhodymenia Palmata (L.)), Alginate, Ammonium, Alginate, Calcium, Alginate, Potassium, Alginate, Sodium, Alginate, Sodium Calcium, Alginic Acid, Alkanet Root, Extract (Alkanna Tinctoria Tausch), Alkanolamide Of Coconut Oil Fatty Acids And Diethanolamine, Alkylene Oxide Adducts Of Alkyl Alcohols/Phosphate Esters Of Same, (Mixture), N-Alkyl(C8-C18 From Coconut Oil) Amine Acetate, Alpha-Alkyl-Omega-Hydroxy-Poly(Oxyethylene), Allspice (Pimenta Officinalis Lindl.), Allspice, Oil (Pimenta Officinalis Lindl.), Allspice, Oleoresin (Pimenta Officinalis Lindl.), Allyl Alpha-Ionone, Allyl Anthranilatc, Allyl Butyrate, Allyl Cinnamate, Allyl Crotonate, Allyl Cyclohexaneacetate, Allyl Cyclohexanebutyrate, Allyl Cyclohexanehexanoate, Allyl Cyclohexanepropionate, Allyl Cyclohexanevalerate, S-Allyl-L-Cysteine, 4-Allyl-2,6-Dimethoxyphenol, Allyl Disulfide, Allyl 2-Ethylbutyrate, Allyl 2-Furoate, Allyl Heptanoate, Allyl Hexanoate, Allyl Hexenoate, Allyl Isothiocyanate, Allyl Isovalerate, Allyl Mercaptan, Allyl Methyl Disulfide, Allyl Methyl Trisulfide, Allyl Nonanoate, Allyl Octanoate, Allyl Phenoxyacetate, Allyl Phenylacetate, Allyl Propionate, Allyl Propyl Disulfide, Allyl Sorbate, Allyl Sulfide, Allyl Thiohexanoate, Allyl Thiopropionate, Allyl Tiglate, Allyl 10-Undecenoate, Allyl Valerate, Almond, Bitter, Oil (Ffpa) (Prunus Spp.), Aloe, Extract (Aloe Spp.), Alpha-Amylcinnamyl isovalerate, Alpha-Ethyl Benzyl Butyrate, Alpha-Hydro-Omega-Hydroxy Poly(Oxyethylene) Poly(Oxypropylene) Poly(Oxyethylene) (15 Mole Minimum) Blocked Copolymer, Low Erucic Acid Rapeseed Oil Polymers, Alpha-Isobutylphenethyl Alcohol, Alpha-Methylbenzyl Butyrate, Alpha-Methylbenzyl Isobutyrate, Alpha-Tocopherol Acid Succinate, Althea Flowers (Althea Officinalis L.), Althea Root (Althea Officinalis L.), Alum (Double Sulfate Of Al And Nh4, K, Or Na), Aluminum Ammonium Sulfate, Aluminum Calcium Silicate, Aluminum Caprate, Aluminum Caprylate, Aluminum Hydroxide, Aluminum Laurate, Aluminum Myristate, Aluminum Nicotinate, Aluminum Oleate, Aluminum Palmitate, Aluminum Potassium Sulfate, Aluminum Salts Of Fatty Acids, Aluminum Sodium Sulfate, Aluminum Stearate, Aluminum Sulfate, Ambergris, Tincture, Ambrette, Absolute, Oil (Hibiscus Abelmoschus L.), Ambrette Seed (Hibiscus Abelmoschus L.), Ambrette Seed, Oil (Hibiscus Abelmoschus L.), Ambrette, Tincture (Hibiscus Abelmoschus L.), 1-Amino-2-Propanol, 2′-Aminoacetophenone, P-Aminobenzoic Acid, 4-Aminobutyric Acid, Dl-(3-Amino-3-Carboxypropypyl)Dimethylsulfonium Chloride, 4-Amino-5,6-Dimethylthieno[2,3-D]Pyrimidin-2 (1 h)-One, 4-Amino-5,6-Dimethyithieno[2,3-D]Pyrimidin-2 (1 h)-One Hydrochloride, 3-[(4-Amino-2,2-Dioxido-1h-2,1,3-Benzothiadiazin-5-Yl)Oxy]-2,2-Dimethl-N-Propylpropanamide, Aminoglycoside 3′-Phosphotransferase Ii, Aminopeptidase From Lactococcus Lactis, Amino Tri(Methylene Phosphoric Acid), Sodium Salt, Ammonia (Also Includes Ammonium Chloride), Ammonium Acetate, Ammonium Bicarbonate, Ammonium Carbonate, Aminonium Caseinate, Ammonium Chloride, Ammonium Citrate, Dibasic, Ammonium Gluconate, Ammonium Hydroxide, Ammonium Isovaterate, Ammonium Pectinate, Ammonium Persulfate, Ammonium Phosphate, Dibasic, Ammonium Phosphate, Monobasic, Ammonium Sulfate, Ammonium Sulfide, Ammonium Sulfite, Amyl Alcohol, Alpha-Amylase Enzyme Preparation From Bacillus Stearothermophilus, Amylase From Aspergillus Flavus, Amylase From Aspergillus Niger, Amylase From Aspergillus Oryzae Amylase From Bacillus Subtilis, Amyl Butyrate, Alpha-Amylcinnamaldehyde, Alpha-Amylcinnamaldehyde Dimethyl Acetal, Alpha-Amylcinnamyl. Acetate, Alpha-Amylcinnamyl Alcohol, Alpha-Amylcinnamyi Formate, Amyl Decanoate, Amyl Formate, Amyl 2-Furoate, Amyl Heptanoate, Amyl Hexanoate, Amyl Isothiocyanate, Amyl Methyl Disulfide, Amyl Octanoate, Amyloglucosidase From Rhizopus Niveus, 2-Amyl-5 Or 6-Keto-1,4-Dioxane, Amyl Salicylate, Amyris (Amyris Balsamifera L.), Amyris, Oil (Amyris Balsamifera L.), (2,4)- And (3,5)- And (3,6)-Dimethyl-3-Cyclohexenylcarbaldehyde, Trans-Anethole, Angelica (Angelica Spp.), Beta-Angelicalactone, Angelica Root (Angelica Spp.), Angelica Root Extract (Angelica Archangelica L.), Angelica Root Oil (Angelica Archangelica L.), Angelica Seed (Angelica Spp.), Angelica Seed Extract (Angelica Archangelica L.), Angelica Seed Oil (Angelica Archangelica L.), Angelica Stem Oil (Angelica Archangelica L.), Angola Weed (Roccella Fuciformis Ach.), Angostura (Galipea Offincinalis Hancock), Angostura. Extract (Galipea Officinalis Hancock), Anisaldehyde Propyleneglycol Acetal, Anise (Pimpinella Anisum L.), Anise Oil (Pimpinella Anisum L.), Anise, Star (Illicium Verum Hook, F.), Star Anise Oil (Illicium Verum Hook, F.), Anisic Acid, Anisole, Anisyl Acetate, Anisyl Alcohol, Anisyl Butyrate, Anisyl Formate, Anisyl Phenylacetate, Anisyl Propionate, Annatto, Extract (Bixa Orellana L.), Annatto, Seed (Bixa Orellana L.), Anoxomer, Sulfonated Anthracite Coal, Beta-Apo-8′-Carotenal, Apple Essence, Apricot Kernel Oil (Prunus Armeniaca L.), Arabinogalactan, L-Arabinose, L-Arginine, Arnica Flowers (Arnica Spp.), Arrowroot Starch, Artemisia (Artemisia Spp.), Artemisia Extract, Artemisia Oil, Artichoke Leaves (Cynara Scolymus L.), Asafetida Fluid Extract (Ferula Assafoetida L.), Asafetida Gum (Ferula Assafoetida L.), Asafetida Oil (Ferula Assafoetida L.), Ascorbic Acid, Ascorbyl Palmitate, Ascorbyl Stearate, L-Asparagine, Asparagus, Seed And Root, Extract, Aspartame, L-Aspartic Acid, Aspergillus Niger For Fermentation Production Of Citric Acid, Astaxatithin, Azodicarbonamide, Bacterial Catalase From Micrococcus Lysodeikticus, Bakers Yeast Extract, Baker's Yeast Glycan, Baker's Yeast Protein, Balm (Melissa Officinalis L.), Balm Leaves (Melissa Officinalis L.), Balm Leaves Extract (Melissa Officinalis L.), Balm Oil (Melissa Officinalis L.), Balsam Fir Needles And Twigs (Abies Balsamea (L) Mill.), Balsam Fir Oil (Abies Balsamea (L.) Mill.), Balsam Fir Oleoresin (Abies Balsamea (L.) MR), Peru Balsam (Myroxylon Pereirae Klotzsch), Peru. Balsam, Oil (Myroxylon Pereirae Klotzsch), Basil (Ocimum Basilicum L.), Basil Bush (Ocimum Minimum L.), Basil Extract (Ocimum Basilicum L.), Basil Oil (Ocimum Basilicum L.), Basil Oleoresin (Ocimum Basilicum L.), Bay (Laurus Nobilis L.), Sweet Bay Leaves Extract (Laurus Nobilis L.), Sweet Bay Leaves Oil (Laurus Nobilis L.), Bay Leaves, West Indian, Extract (Pimenta Acris Kostel), Bay Leaves, West Indian, Oil (Pimenta Racemosa (Mill.) J. V. Moore), Bay Leaves, West Indian, Oleoresin (Pimenta Acris Kostel), Beechwood, Creosote (Fagus Spp.), Beeswax, Bleached Beeswax, Bentonite, Benzaldehyde, Benzaldehyde Dimethyl Acetal, Benzaldehyde Glyceryl Acetal, Benzaldehyde Propylene Glycol Acetal, Benzene, Benzenethiol, 2-Benzofurancarboxaldehyde, Benzoic Acid, Benzoin, Benzoin, Resin (Styrax Spp.), Benzophenone, Benzothiazole, N-Benzoyianthranilic Acid, Benzoyl Peroxide, Benzyl Acetate, Benzyl Acetoacetate, Benzyl Alcohol, Benzyl Benzoate, Benzyl Butyl Ether, Benzyl Butyrate, Benzyl Cinnamate, Benzyl 2,3-Dimethylerotonate, Benzyl Disulfide, Benzyl Ethyl Ether, Benzyl Formate, 3-Benzyl-4-Heptanone, Benzyl Hexanoate, Benzyl Isobutyrate, Benzyl Isothiocyanate, Benzyl Isovalerate, Benzyl Levulinate, Benzyl Mercaptan, Benzyl Methoxyethyl Acetal, Benzyl Trans-2-Methyl-2-Butenoate, Benzyl Methyl Sulfide, Benzyl Nonanoate, Benzyl Phenylacetate, Benzyl Propionate, Benzyl Salicylate, Bergamot, Oil (Citrus Aurantium L. Subsp. Bergamia Wright Et Arn.), Betaine, Biotin, Biphenyl, Birch, Sweet, Oil (Betula Lenta L.), Birch Tar, Oil (Betula Pendula Roth And Related Betula Spp.), Bisabolene, Bis(2,5-Dimethyl-3-Furyl) Disulfide, Bis(2-Methyl-3-Furyl) Disulfide, Bis(1-Mercaptopropyl)Sulfide, Bis-(Methylthio)Methane, Bis(2-Methylphenyl) Disulfide, Bis(2-Methyl-3-Furyl) Tetrasulfide, Blackberry Bark, Extract (Rubus, Spp. Of Section Eubatus), Blackberry Fruit Extract, Bois De Rose, Oil (Aniba Rosaeodora Ducke), Boldus Leaves (Peumus Boldus Mol.), Bonito, Dried, Borax, Boric Acid, Borneol, L-Bornyl. Acetate, Bornyl Acetate, Bornyl Butyrate, Bornyl Formate, Bornyl Isovalerate, Bornyl Valerate, Boronia, Absolute (Boronia Megastigma Nees), Bouillon, Vegetable, Smoke, Beta-Bourbonene, Bromelain, Brominated Vegetable Oil, Bryonia Root (Bryonia Spp.), Buchu Leaves (Barosma Bettaina And Crenulata), Buchu Leaves Extract, Buchu Leaves, Oil (Barosma Spp.), Buckbean Leaves (Menyanthes Trifoliata L.), Buckbean Leaves, Extract (Menyanthes Trifoliata L.), Butadiene-Styrene Rubber, 4-(Methylthio)Butanal, Butanal Dibenzyl Thioacetal, N-Butane, 1,3-Butanedithiol, 1,2-Butanedithiol, 2,3-Butanedithiol, 1-Butanethiol, 2-Butanol, 2-Butanone, Butan-3-One-2-Y1 Butanoate, (E)-2-Butenoic Acid, 4-(2-Butenylidene)-3,5,5-Trimethylcyclohex-2-En-1-One, 3-Butenyl Isothiocyanate, 1-Buten-1-Y1 Methyl Sulfide, Butter Acids, Butter Esters, Butter Fat, Enzyme-Modified, With Added Butyric Acid, Butter Starter Distillate, Butyl Acetate, Butyl Acetoacetate, Butyl Alcohol, Butylamine, Sec-Butylamine, Butyl Anthranilate, Butylated Hydroxyanisole, Butylated Hydroxytoluene, 2-Butyl-2-Butenal, Butyl Butyrate, Butyl Butyryllactate, Alpha-Butylcinnamaldehyde, Butyl Cinnamate, Butyl 2-Decenoate, 1,3-Butylene Glycol, Butyl Ethyl Disulfide, Butyl Ethyl Malonate, Butyl Formate, 2-Butylfuran, Butyl Heptanoate, Butyl Hexanoate, Butyl P-Hydroxybenzoate, Alpha-Butyl-Omega-Hydroxypoly(Oxyethytene) Poly(Oxypropylene), 3-Butylidenephthalide, Butyl Isobutyrate, 2-Butylisothiocyanate, Butyl Isothiocyanate, Butyl Isovalerate, Butyl Lactate, Butyl Laurate, Butyl Levulinate, N-Butyl 2-Methylbutyrate, Butyl Beta-(Methylthio)Acrylate, Butyl Beta-Naphthyl Ether, Butyl Oleate Sulfate, 2-Butyl-5 Or 6-Keto-1,4-Dioxane, Butyl Phenylacetate, 3-N-Butylphthalide, Butyl Propionate, Butyl Salicylate, Butyl Stearate, Butyl Sulfide, Butyl 10-Undecenoate, Butyl Valerate, Butyraldehyde, Butyramide, Butyric Acid, 2-Butyrylfuran, Cadinene, Caffeine, Cajeput, Oil (Melaleuca Leucadendron L.), Calcium Acetate, Calcium Ascorbate, Calcium Benzoate, Calcium Bromate, Calcium Caprate, Calcium Caprylate, Calcium Carbonate, Calcium Caseinate, Calcium Chloride, Calcium Citrate, Calcium Diglutamate, Calcium Fumarate, Calcium Gluconate, Calcium Glycerophosphate, Calcium Hexametaphosphate, Calcium Hydroxide, Calcium Hypophosphite, Calcium Iodate, Calcium Lactate, Calcium Lactobionate, Calcium Laurate, Calcium Lignosulfonate, Calcium Myristate, Calcium Oleate, Calcium Oxide, Calcium Palmitate, Calcium Pantothenate, Calcium Pantothenate, Calcium Chloride Double Salt, Calcium Peroxide, Calcium Phosphate, Dibasic, Calcium Phosphate, Monobasic, Calcium Phosphate, Tribasic, Calcium Phytate, Calcium Propionate, Calcium Pyrophosphate, Calcium Salts Of Fatty Acids, Calcium Silicate, Calcium Sorbate, Calcium Stearate, Calcium Stearoyl-2-Lactylate, Calcium Sulfate, Calumba Root (Jatrorrhiza Palmata (Lam.) Miers), Calumba Root, Extract (Jatrorrhiza Palmata (Lam.) Miers), Camphene, Campholene Acetate, Alpha-Campholenic Alcohol, D-Camphor, Camphor, Japanese, White, Oil (Cinnamomum Camphora (L.) Nees .Et Eberm,), Camphor Oil, Formosan Ho-Sho, Leaves (Cinnamomum Camphora), Cananga, Oil (Cananga Odorata Hook. F. And Thoms.), Candetilla Wax (Wax From Stems And Branches Of Euphorbia Cerifera), Candida Guilliermondii, Candida Lipolytica, Canthaxanthin, Capers (Capparis Spinosa. L.), Caprolactam, Capsicum (Capsicum Spp.), Capsicum Extract (Capsicum Spp.), Capsicum, Oleoresin (Capsicum Spp.), Caramel, Caraway (Carom Carvi L.), Caraway, Black (Nigella Sativa L.), Caraway, Oil (Carum Carvi L.), Carbohydrase And Cellulase From Aspergillus Niger, Carbohydrase And Protease, Mixture, From Bacillus Subtilis, Carbohydrase From Aspergillus Oryzae, Carbohydrase From Bacillus Amyloliquefaciens, Carbohydrase From Bacillus Licheniformis, Carbohydrase From Bacillus Subtilis, Carbohydrase From Rhizopus Oryzae, Carbohydrase From Saccharomyces Spp., Carbohydrase/Proteinase Preparation, Bacillus Licheniformis, Carbon Dioxide, Carboxymethyl Cellulose, Carboxymethyl Cellulose, Sodium Salt, Carboxymethyl Hydroxyethyl Cellulose, Cardamom (Elletaria Cardamomum (L.) Maton), Cardamom Oleoresin, Cardamom Seed, Oil (Elletaria Cardamomum (L.) Maton), 3-Carene, Carmine (Coccus Cacti L.), Carnauba Wax (Copernicia Cerifera (Arruda) Mart.), L-Carnitine, Carob Bean, Extract (Ceratonia Siliqua L.), Beta-Carotene, Carrageenan, Carrageenan, Ammonium Salt Of, Carrageenan, Ammonium Salt Of, With Polysorbate 80, Carrageenan And Salts Of Carrageenan, Carrageenan, Calcium Salt Of, Carrageenan, Calcium Salt Of, With Polysorbate 80, Carrageenan, Potassium Salt Of, Carrageenan, Potassium Salt Of, With Polysorbate 80, Carrageenan Salts With Polysorbate 80, Carrageenan, Sodium Salt Of, Carrageenan, Sodium Salt Of, With Polysorbate 80, Carrageenan With Polysorbate 80, Carrot (Daucus Carota L.), Carrot, Dehydrated, Carrot, Extract, Carrot, Oil (Daucus Carota L.), Carvacrol, Carvacryl Ethyl Ether, Carveol, 4-Carvomenthenol, Carvomenthol, Carvone, Cis-Carvone Oxide, Carvyl Acetate, Carvyl Palmitate, Carvyl Propionate, Beta-Caryophyllene, Beta-Caryophyllene Alcohol, Caryophyllene Alcohol, Beta-Caryophyllene Alcohol Acetate, Catyophyllene Alcohol Acetate, Beta-Caryophyllene Oxide, Cascara, Bitterless, Extract (Rhamnus Purshiana DC,), Cascarilla Bark, Extract (Croton Spp.), Cascarilla Bark, Oil (Croton Spp.), Casein, Cassia Buds (Cinnamomum Cassia Blume), Cassie, Absolute (Acacia Farnesiana (L.) Willd.), Castoreum, Extract (Castor Spp.), Castoreum, Liquid (Castor Spp.), Castor Oil (Ricinus Communis L.), Catalase From Aspergillus Niger, Catalase From Bovine Liver, Catalase From Penicillium Notatum, Catechu, Black, Extract (Acacia Catechu Willd.), Catechu, Black, Powder (Acacia Catechu Willd.), Cedar Leaf Oil (Thuja Occidentals L.), Cedarwood Oil Alcohols, Cedarwood Jit Terpenes, (+)-Cedrol, Cedryl Acetate, Celery Seed (Apium Graveolens L.), Celery Seed, Extract (Apium Graveolens L.), Celery Seed, Extract Solid (Apium Graveolens L.), Celery Seed, Oil (Apium Graveolens L.), Celery Seed, Oleoresin, Cellulose From Trichoderma Longibrachiatum, Cellulose Acetate, Cellulose, Diethylaminoethyl, Cellulose, Methyl, Cellulose, Methyl Ethyl, Cellulose, Microcrystalline, Cellulose Triacetate, Centaury (Centaurium Umbellatum Gilib.), Cereal Solids, Hydrolyzed, Cetyl Alcohol, Chamomile Flower (Matricaria Chamomilia L.), Chamomile Flower (Anthemis Nobilis L.), Chamomile Flower, Hungarian, Oil (Matricaria. Chamomilla L.), Chamomile Flower, Oil (Anthemis Nobilis L.), Chamomile Flower, Roman, Extract (Anthemis Nobilis L.), Char Smoke Flavor, Cherry Bark, Wild, Extract (Prunus Serotina Ehrh.), Cherry-Laurel Leaves (Prunus Laurocerasus L.), Cherry Laurel, Oil (Prunus Laurocerasus L.) (Ffpa), Cherry-Laurel Water (Prunus Laurocerasus L.), Cherry Pits, Extract (Prunus Spp.), Chervil (Anthriscus Cerefolium (L.) Hoffm.), Chervil, Extract (Anthriscus Cerefolium L.), Chestnut Leaves (Castanea Dentata (Marsh.) Borkh.), Chestnut Leaves, Extract (Castanea Dentata (Marsh.) Borkh.), Chestnut Leaves, Extract Solid (Castanea Dentata (Marsh.) Borkh.), Chicle (Manilkara Zapotilla Gilly And M. Chicle Gilly), Chicle, Venezuelan (Manilkara Williamsii Standley And Related Spp.), Chicory, Extract (Cichorium Intybus L.), ChiIte (Cnidoscolus (Also Known As Jatropha) Spp.), Chiquibul (Manilkara Zapotilla Gilly), Chirata (Swertia Chirata Buch.-Ham.), Chirata, Extract (Swertia Chirata Buch.-Ham.), Chives (Allium Schoenoprasum L.), Chlorine, Chlorine Dioxide, Chlorine Solution, Aqueous, Chloroform, Chloromethyl Methyl Ether, Chloropentafluoroethane, Chlorophyll, Cholic Acid, Choline Bitartrate, Choline Chloride, Choline Chloride (Also Includes Choline), Chrysanthemum Extract, Chymosin Preparation, Aspergillus Niger Var. Awamori, Chymosin Preparation, Escherichia Coli K-12, Chymosin Preparation, Kluyveromyces Marxianus Var. Lactis, Cinchona Bark, Red (Cinchona Succirubra Pav. Or Its Hybrids), Cinchona Bark, Red, Extract (Cinchona Succirubra Pav. Or Its Hybrids), Cinchona Bark, Yellow (Cinchona Spp.), Cinchona Bark, Yellow, Extract (Cinchona Spp.), Cinchona, Extract (Cinchona Spp.), 1,4-Cineole, Cinnamaldehyde, Cinnamaldehyde Ethylene Glycol Acetal, Cinnamaldehyde Propyleneglycol Acetal, Cinnamic Acid, Cinnamon (Cinnamomum Spp.), Cinnamon Bark, Extract (Cinnamomum Spp.), Cinnamon Bark, Oil (Cinnamomum Spp.), Cinnamon Bark Oleoresin, Ceylon, Chinese, Or Saigon (Cinnamomum Spp.), Cinnamon Leaf, Oil (Cinnamomum Spp.), Cinnamon Leaf Oil, Rectified, Cinnamyl Acetate, Cinnamyl Alcohol, Cinnamyl Benzoate, Cinnamyl Butyrate, Cinnamyl Cinnamate, Cinnamyl Formate, Cinnamyl Isobutyrate, Cinnamyl Isovalerate, Cinnamyl Phenylacetate, Cinnamyl Propionate, Cis- And Trans-Ethyl 2,4-Dimethyl-1,3-Dioxolane-2-Acetate, Cis-And Trans-5-Ethyl-4-Methyl-2-(2-Butyl)-Thiazoline, Cis And Trans-5-Ethyl-4-Methyl-2-(2-Methylpropyl)-Thiazoline, Cis- And Trans-2-Isobutyl-4-Methyl-1,3-Dioxolane, Cis- And Trans-2-Isopropyl-4-Methyl-1,3-Dioxolane, Cis- And Trans-L-Mercapto-P-Menthan-3-One, (+−)-Cis- And Trans-2-Methyl-2-(4-Methyl-3-Pentenyl)Cyclopropanecarbaldehyde, (+/−)Cis-And Trans-2-Pentyl-4-Propyl-1,3-Oxathiane, Cis-4-Decenol, Cis-4-Heptenal, Cis-3-Hexenoic Acid, Cis-3-Hexen-1-O1, Cis-3-Hexen-1-Y1 Acetate, Cis-3-Hexenyl Acetoacetate, Cis-3-Hexenyl Butyrate, Cis-3-Hexenyl Cis-3-Hexenoate, Cis-3-Hexenyl Formate, Cis-3-Hexenyl Hexanoate, Cis-5-Isopropenyl-Cis-2-Methylcyclopentan-1-Carboxaldehyde, Cis-3-Nonen-1-O1, Cis-9-Octadecenyl Acetate, Cis-5-Octenoic Acid, Cis-4-Octenol, Cis-2-Octenol, Citral, Citral Diethyl Acetal, Citral Dimethyl. Acetal, Citral Glyceryl Acetal, Citral Propylene Glycol Acetal, Citric Acid, Citric And Fatty Acid Esters Of Glycerol, Citronellal, Citronella, Oil (Cymbopogon Nardus Rendle), Citronelloxyacetaldehyde, Citronellyl Acetate, Citronellyl Anthranilate, Citronellyl Butyrate, Citronellyl Formate, Citronellyl Isobutyrate, Citronellyl Phenylacetate, Citronellyl Propionate, Citronellyl Trans-2-Methyl-2-Butenoate, Citronellyl Valerate, Citrus Peels, Extract (Citrus Spp.), Citrus Red No. 2, Civet, Absolute (Viverra Civetta Schreber And Viverra Zihetha Schreber), Clary (Salvia Sclarea L.), Clary Sage, Absolute, Clary, Oil (Salvia Sclarea L.), Clary Sage, Concrete, Clay, Attapulgite, Clove Bud, Extract (Eugenia Spp.), Clove Bud, Oil (Eugenia Spp.), Clove Bud, Oleoresin (Eugenia Spp.), Clove Leaf, Oil (Eugenia Spp.), Clover (Trifolium Spp.), Clover, Extract (Trifolium Spp.), Clover Herb Distillate, Clover, Oil (Trifolium Spp.), Clover Tops, Red, Extract Solid (Trifolium Pratense L.), Cloves (Eugenia Spp.), Clove Stem, Oil (Eugenia Spp.), Coca Leaf, Extract (Decocainized) (Erythroxylon Coca Lam), Cochineal Extract (Coccus Cacti L.), Cocoa Butter Substitute From Coconut Oil, Palm Kernel Oil Or Both Oils, Cocoa Butter Substitute From Palm Oil, Cocoa Butter Substitute Primarily From High-Oleic Safflower Or Sunflower Oil, Cocoa Extract, Cocoa With Dioctyl Sodium Sulfosuccinate, Coconut Oil, Coconut Oil, Refined, Coffee Concentrate, Pure, Coffee Extract (Coffea Spp.), Coffee Extract, Solid, Cognac, Green, Oil, Cognac, White, Oil, Collagen, Combustion Product Gas, Copaiba (South American Spp. Of Copaifera L.), Copaiba, Oil (South American Spp. Of Copaifera L.), Copals, Manila, Copper Gluconate, Copper Sulfate, Coriander (Coriandrum Sativum L.), Coriander Leaf Oil (Coriandrum Sativum L.), Coriander, Oil (Coriandrum Sativum L.), Cork, Oak (Quercus Spp.), Corn Endosperm Oil, Corn Gluten, Corn Mint Oil, Corn Silk, Corn Silk Extract (Zea Mays Corn Silk, Oil (Zea Mays L.), Cornstarch, Cornstarch, Waxy, Corn Steep Liquor, Corn Syrup, Costrnary (Chrysanthemum Balsamita L), Costus Root, Oil (Saussurea Lappa Clarke), Cottonseed Flour, Defatted, Cottonseed Flour, Partially Defatted, Cooked, Cottonseed Flour, Partially Defatted, Cooked, Toasted, Cottonseed Kernels, Glandless, Raw, Cottonseed Kernels, Glandless, Roasted, Coumarone-Indene Resins, M-Cresol, P-Cresol, O-Cresol, Crown Gum, Cubeb (Piper Cubeba L. F.), Cubeb, Oil (Piper Cubeba L F.), Cubebol, Cumin (Cuminum Cyminum L.), Cuminaldehyde, Cumin, Oil (Cuminum Cyminum L.), Cuprous Iodide, Curdlan, Currant Buds, Black, Absolute (Ribes Nigrum L), Currant Juice, Black, Currant Leaves, Black (Ribes Nigrum L.) Beta-Cyclodextrin, Cycloheptadeca-9-En-1-One, Cyclohexane, Cyclohexaneacetic Acid, Cyclohexanecarboxylic Acid, Cyclohexaneethyl Acetate, Cyclohexanone, Cyclohexanone Diethyl Ketal, 2-Cyclohexenone, Cyclohexyl Acetate, Cyclohexylamine, Cyclohexyl Anthranilate, Cyclohexyl Butyrate, Cyclohexyl Cinnamate, Cyclohexyl Formate, Cyclohexyl Isovalerate, Cyclohexylmethyl Pyrazine, Cyclohexyl Propionate, Cycloionone, Cyclopentanethiol, Cyclopentanone, 2-Cyclopentylcyclopentanone, Cyclopropanecarboxylic Acid (2-Isopropyl-5-Methyl-Cyclohexyl)-Amide, N-Cyclopropyl-5-Methyl-2-Isopropylcyclohexanecarboxamide, N-Cyclopropyl-Trans-2-Cis-6-Nonadienamide, Cyclotene Butyrate, Cyclotene Propionate, P-Cymene, L-Cysteine, L-Cysteine Monohydrochloride, Dl-Cystine, L-Cystine, Daidai Peel Oil, Damar Gum (Shorea Dipterocarpaceae), Alpha-Damascone, Delta-Damascone, Trans-Alpha-Damascone, Damiana Leaves (Turnera Diffusa Willd.) Dandelion, Fluid Extract (Taraxacum Spp.), Dandelion Root, Extract Solid (Taraxacum Spp.), Davana Oil (Artemesia Pallens Wall.) 2-Trans,4-Trans-Decadienal, (E,E)-2,4-Decadien-1-O1, Delta-Decalactone, Gamma-Decalactone, Decalepis Hamiltonii Extract, Decanal, Decanal Dimethyl Acetal, Decanal Propyleneglycol Acetal, Decanoic Acid, 1-Decanol, 3-Decanol, 3-Decanone, 2-Decanone, 2-Trans-4-Trans-7-Cis-Decatrienal, 9-Decenal, 2-Decenal, 4-Decenal, 4-Decenoic Acid, (E)-2-Decenoic Acid, 9-Deeenoic Acid, 6-Decenoic Acid, 5-Decenoic Acid, 1-Decen-3-O1, 8-Decen-5-Olide, 9-Decen-5-Olide, 7-Decen-4-Olide, 9-Decen-2-One, 3-Decen-2-One, 6-[5(6)-Decenoyloxy]Decanoic Acid, 4-Decenyl Acetate, Cis-, Decyl Acetate, Decyl Butyrate, 2-Decylfuran, Decyl Propionate, Deertongue Solid Extract, Dehydrated Beets, Dehydroacetic Acid, Dehydrodihydroionol, Dehydrodihydroionone, Debydromenthofurolactone, Dehydronootkatone, 8,9-Dehydrotheaspirone, Desoxycholic Acid, Dextrans (Avg M W Less Than 100,000), Dextrin, Dextrose, Diacetyl, Di N-Alkyl(C8-C18 From Coconut Oil) Dimethyl Ammonium Chloride, Diallyl Polysulfides, Diallyl Trisulfide, Diamyl Ketone, Diastase From Aspergillus Oryzac, Diatomaceous Earth, Dibenzyl Ether, 2,2-Dibromo-3-Nitrilopropionamide, 4,4-Dibutyl-Gamma-Butryolactone, Dibutyl Sebacate, Dichlorodifluoromethane, Dicyclohexyl Disulfide, Diethanolamide Condensate From Soybean Oil Fatty Acids (C16-C18), Diethanolamide Condensate From Stripped Coconut Oil Fatty Acids (C10-C18), Diethylaminoethanol, Diethyl Disulfide, Diethylene Glycol Distearate, Diethylenetriamine, Diethylenetriamine Crosslinked With Epichlorohydrin, Diethyl Malate, Diethyl Malonate, 3,5-Diethyl-2-Methylpyrazine, 2,5-Diethyl-3-Methylpyrazine, 2,3-Diethyl-5-Methylpyrazine, 2,3-Diethylpyrazine, Diethyl Sebacate, Diethyl Succinate, Diethyl Sulfide, Diethyl Tartrate, 2,5-Diethyltetrahydrofuran, Diethyl Trisulfide, (+/−)-Cis- And Trans-3,5-Diethyl-1,2,4-Trithiolane, Difurfuryl Ether, 2,4-Difurfurylfuran, Di-2-Furylmethane, Digeranyl Ether, Dihydro-Alpha-Ionone, Dihydrocarveol, Cis-Dihydrocarvone, Dihydrocarvyl Acetate, Dihydrocoumarin, 6,7-Dihydro-2,3-Dimethyl-5h-Cyclopentapyrazine, 4,5-Dihydro-2,5-Dimethyl-4-Oxo-3-Furanyl Butyrate, (+/−)-Dihydrofarnesol, Dihydrogalangal Acetate, Dihydro-Beta-Ionol, Dihydro-Beta-Ionone, 3,6-Dihydro-4-Methyl-2(2-Methylpropen-1-Yl)-2h-Pyran, 5,7-Dihydro-2-Methylthieno (3,4-D)Pyrimidine, (+/−)-Dihydromintlactone, Dihydronootkatone, (+/−)-Cis- And Trans-1,2-Dihydroperillaldehyde, 4,5-Dihydro-3(2h)Thiophenone, Dihydro-2,4,6-Trimethyl-4h-1,3,5-Dithiazine, Dihydro-2,4,6-Tris(2-Methylpropyl)-4h-1,3,5-Dithiazine, Dihydroxyacetone (Dimer), Dihydroxyacetone (Monomer), Dihydroxyacetophenone, 2,4-Dihydroxybenzoic Acid, 3,4-Dihydroxybenzoic Acid, 2,5-Dihydroxy-1,4-Dithiane, 5,7-Dihydroxy-2-(3-Hydroxy-4-Methxy-Phenyl)-Chroman-4-One, 3′,7-Dihydroxy-4′-Methoxyflavan, Diisoamyl Disulfide, Diisoamyl Trisulfide, Diisobutyl Adipate, Diisobutyl Ketone, Diisopentyl Thiomalate, Diisopropyl Adipate, Diisopropyl Disulfide, Diisopropyl Trisulfide, Dilauryl Thiodipropionate, Dill (Anethum Graveolens L.), Dill, Oil (Anethum Graveolens L.), Dill Seed, Indian (Anethum Spp.), Dill Seed Oil (Anethum Sowa Roxb.), Dimenthyl Glutarate, Dimercaptomethane, M-Dimethoxybenzene, P-Dimethoxybenzene, 1,2-Dimethoxybenzene, N1-(2,4-Dimethoxybenzyl)-N2-(2-(Pyridin-2-Y1) Ethyl)Oxalamide, 1,1-Dimethoxyethane, 1,1-Dimethoxy-Trans-2-Hexene, 2,6-Dimethoxyphenol, N-[2-(3,4-Dimethoxyphenyl)Ethyl]-3,4-Dimethoxycinnamic Acid Amide, 3,4-Dimethoxy-1-Vinylbenzene, 3,6-Dimethyl-2,3,3a,4,5,7a-Hexahydrobenzofuran, 2,4-Dimethylacetophenone, 1,4-Dimethyl-4-Acetyl-1-Cyclohexene, 2,4-Dimethyl-5-Acetylthiazole, Dimethyl Adipate, Dimethylamine, Dimethylamine-Epichlorohydrin Copolymer, 2,4-Dimethylanisole, 2,4-Dimethylbenzaldehyde, 2,3-Dimethylbenzofuran, P,Alpha-Dimethylbenzyl Alcohol, Dimethylbenzyl Carbinyl Crotonate, Dimethylbenzyl Carbinyl Hexanoate, Alpha,Alpha-Dimethylbenzyl Isobutyrate, 3,5-Dimethyl-1,2-Cyclopentadione, 3,4-Dimethyl-1,2-Cyclopentadione, (+/−)-Trans- And Cis-5-(2,2-Dimethylcyclopropyl)-3-Methyl-2-Pentenal, Dimethyl Dialkyl Ammonium Chloride, Dimethyl Dicarbonate, 2(3),5-Dimethyl-6,7-Dihydro-5h-Cyclopentapyrazine, 2,5-Dimethyl-2,5-Dihydroxy-1,4-Dithiane, 2,4-Dimethyl-1,3-Dioxolane, Dimethylethanolamine, 2,5-Dimethyl-4-Ethoxy-3(2h)-Furanone, 2,5-Dimethyl-4-Ethyloxazole, 4-(1,1-Dimethylethyl)Phenol, 4,5-Dimethyl-2-Ethyl-3-Thiazoline, 2,5-Dimethylfuran, 2,5-Dimethyl-3(2h)-Furanone, 2,5-Dimethyl-3-Furanthiol, 2,5-Dimethyl-3-Furanthiol Acetate, 2,6-Dimethyl-4-Heptanol, 2,6-Dimethyl-3-((2-Methyl-3-Furyl)Thio)-4-Heptanone, 2,6-Dimethyl-5-Heptenal, 2,6-Dimethyl-5-Heptenal Propyleneglycol Acetal, 2,6-Dimethyl-6-Hepten-1-O1, 4,5-Dimethyl-3-Hydroxy-2,5-Dihydrofuran-2-One, 3,6-Dimethyl-2-Isobutylpyrazine, 3,5-Dimethyl-2-Isobutylpyrazine, 4,5-Dimethyl-2-Isobutyithiazole, 4,5-Dimethyl-2-Isobutyl-3-Thiazoline, 3-(1-((3,5-Dimethylisoxazol-4-Y1)Methyl)-1h-Pyrazol-4-Y1)-1-(3-Hydroxybenzyl)-5,5-Dimethylimidazolidine-2,4-Dione, 3-(1-((3,5-Dimethylisoxazol-4-Y1)Methyl)-1h-Pyrazol-4-Y1)-1-(3-Hydroxybenzyl)Imidazolidine-2,4-Dione, (+/−)-N,N-Dimethyl Menthyl Succinamide, 2,5-Dimethyl-3-Mercaptotetrahydrofuran, 2,5-Dimethyl-4-Methoxy-3(2h)-Furanone, 2,6-Dimethyl-10-Methylene-2,6,1-Dodecatrienal, 3,9-Dimethyl-6-(1-Methylethyl)-1,4-Dioxaspiro[4.5] Decan-2-One, 2,2-Dimethyl-5-(1-Methylpropen-1-Y1) Tetrahydrofuran, (+/−)-Trans- And Cis-4,8-Dimethyl-3,7-Nonadien-2-O1, 4,8-Dimethyl-3,7-Nonadien-2-One, Cis-, 4,8-Dimethyl-3,7-Nonadien-2-One, 4,8-Dimet hyl-3,7-Nonadien-2One, Trans-, (+/−)-Trans- And Cis-4,8-Dimethyl-3,7-Nonadien-2-Y1 Acetate, 2,4-Dimethyl-4-Nonanol, (E)-2-(3,7-Dimethyl-2,6-Octadienyl)Cyclopentanone, N-3,7-Dimethyl-2,6-Octadienylcyclopropylcarboxamide, 2-Trans-3,7-Dimethylocta-2,6-Dienyl 2-Ethylbutanoate, 2,6-Dimethyloctanal, 3,7-Dimethyloctanal, 3,7-Dimethyl-1-Octanol, (E)-3,7-Dimethyl-1,5,7-Octatrien-3-O1, 3,7-Dimethyl-6-Octenoic Acid, 2,4-Dimethyl-3-Oxazoline, 2,4-Dimethyl-2-Pentenoic Acid, Alpha,Alpha-Dimethylphenethyl Acetate, Alpha,Alpha-Dimethylphenethyl Alcohol, N,N-Dimethylphenethylamine, Alpha,Alpha-Dimethylphenethl Butyrate, Alpha,Alpha-Dimethylphenethyl Formate, Dimethylpolysiloxane, 2,5-Dimethylmazine, 2,6-Dimethylpyrazine, 2,3-Dimethylpyrazine, 2,6-Dimethylpyridine, 2,4-Dimethylpyridine, 2,5-Dimethylpyrrole, P,Alpha-Dimethylstyrene, Dimethyl Succinate, Dimethyl Sulfoxide, 2,5-Dimethylthiazole, 4,5-Dimethylthiazole, 2-(2-Butyl)-4,5-Dimethyl-3-Thiazoline, 2,5-Dimethyl-3-Thioisovaleryfuran, 3,4-Dimethylthiophene, 2,6-Dimethylthiophenol, Dimethyl Trisulfide, 3,5-Dimethyl-1,2,4-Trithiolane, 2,4-Dimethyl-5-Vinyithiazole, Dioctyl Adipate, Dioctyl Sodium Sulfosuccinate, Diphenyl Ether, 1,3-Diphenyl-2-Propanone, Dipotassium Phosphate, 1,1-Dipropoxyethane, Dipropyl Adipate, Dipropyl Trisulfide, Di-Sec-Butyl Disulfide, Disodium Citrate, Disodium Cyanodithioimidocarbonate, Disodium Ethylenebisdithiocarbamate, Disodium Guanylate, Disodium Inosinate, Disodium Succinate, Di -(Butan-3-One-1-Yl) Sulfide, 2,5-Dithiahexane, 1,4-Dithiane, 2,8-Dithianon-4-En -4-Carboxaldehyde, 2,2′-(Dithiodimethylene) Difuran, Dittany Of Crete (Origanum Dictamnus L.), Dittany (Fraxinella) Roots (Dictamnus Albus L.), Divanillin, D1-Camphor, D-Limonen-10-O1, D1-Isoleucine, D1-Isomenthone, D1-Valine, Epsilon-Dodecalactone, Gamma-Dodecalactone, Delta-Dodecalactone, Dodecanal Dimethyl Acetal, (Z)-4-Dodecenal, 2-Dodecenal, 11-Dodecenoic Acid, 9-Dodecen-5-Olide, Dodecyl Butyrate, Dodecyl Gallate, Dodecyl Isobutyrate, Dodecyl Lactate, N-Dodecylmercaptan, Alpha-(P-Dodecylphenyl)-Omega-Hydroxypoly(Oxyethylene), Dodecyl Propionate, Dog Grass, Extract (Agropyron Repens (L.) Beauv.), D-8-P-Menthene-1,2-Epoxide, Dragon's Blood, Extract (Daemonorops Spp. Or Other Botanical Sources), Dried Algae Meal, Edta, Calcium Disodium, Edta, Disodium, Edta, Disodium Iron, Edta, Tetrasodium, (2e,6e,8e)-N-(2-Methylpropyl)-2,6,8-Decatrienamide, Egg White Lysozyme, Elder Flowers (Sambucus Canadensis L. Or Sambucus Nigra L.), Elder Flowers, Extract (Sambucus Canadensis L. Or Sambucus Nigra L.), Elder Tree Leaves (Sambucus Nigra L.), Elecampane Root, Extract (Inula Helenium L.), Elecampane Root, Oil (Inula Helenium L.), Elemi, Gum, Elemi, Oil (Canarium Spp.), Enzyme-Modified Fats, Enzyme modified dairy ingredients, Enzymes, Bacterial, Enzymes, Proteolytic, Epichlorohydrin Crosslinked With Ammonia, (+/−)-2,8-Epithio-Cis-P-Menthane, 2,3-Epoxydecanal, 4,5-Epoxy-(E)-2-Decenal, 2,3-Epoxyheptanal, 2,3-Epoxyoctanal, Epoxyoxophorone, Epsilon-Decalactone, Erigeron, Oil (Erigeron Canadensis L.), (?)-Eriodictyol, Erythorbic Acid, Esterase-Lipase From Mucor Miehei, Estragole, 1,2-Ethanedithiol, Ethane-1,1-Dithiol, Ethanesulfonic acid 2-(1-Difluoro-((Trifluoroethenyl)Oxy)Methyl)-1,2,2,2-Tetrafluoroethoxy)-1,1,2,2-Tetrafluoro-, Polymer With Tetrafluoroethane, Ethanethiol, P-Ethoxybenzaldehyde, N-[(Ethoxycarbonyl)Methyl)-P-Menthane-3-Carboxamide, 2-Ethoxy-3-Ethylpyrazine, 2-Ethoxy-3-Isopropylpyrazine, 1-Ethoxy-3-Methyl-2-Butene, O-(Ethoxymethyl)Phenol, Ethoxyquin, 2-Ethoxythiazole, (+−)-Ethyl 3-Hydroxy-2-Methylbutyrate, Ethyl Abietate, Ethyl (P-Totyloxy)Acetate, Ethyl Acetate, Ethyl 2-(Methylthio)Acetate, Ethyl Acetoacetate, Ethyl Acetoacetate Ethyleneglycol Ketal, (+/−)-Ethyl 3-Acetoxy-2-Methylbutyrate, Ethyl 5-Acetoxyoctanoate, S-Ethyl 2-Acetylaminoethanethioate, Ethyl Alpha-Acetylcinnamate, Ethyl 2-Acetylhexanoate, Ethyl 2-Acetyloctanoate, Ethyl 2-Acetyl-3-Phenylpropionate, 1-Ethyl-2-Acetylpynote, Ethyl 4-(Acetylthio)Butyrate, Ethyl Aconitate (Mixed Esters), Ethyl Acrylate, Ethyl Alcohol, Ethylamine, Ethyl P-Anisate, Ethyl Anthranilate, 4-Ethylbenzaldehyde, Ethyl Benzoate, Ethyl Benzoylacetate, Ethyl Brassylate, 2-Ethylbutyl Acetate, 2-Ethylbutyraldehyde, Ethyl Butyrate, Ethyl 3-(Methylthio)Butyrate, 2-Ethylbutyric Acid, Ethyl Cellulose, Ethyl Cinnamate, Ethyl Crotonate, Ethyl Cyclohexanecarboxylate, Ethyl Cyclohexanepropionate, Ethyl Trans-2,Cis-4-Decadienoate, Ethyl Decanoate, Ethyl 2,4,7-Decatrienoate, Ethyl Trans-2-Decenoate, Ethyl Trans-4-Decenoate, Ethyl 4,5-Dihydro-2,5-Dimethyl-4-Oxo-3-Furancarboxylate, 2-Ethyl-2,5-Dihydro-4-Methytthiazole, 4-Ethyl-2,6-Dimthoxyphenol, 2(4)-Ethyl-4(2),6-Dimethyldihydro-1,3,5-Dithiazine (Mixture Of Isomers), 2-Ethyl-4,5-Dimethyloxazole, 3-Ethyl-2,6-Dimethylpyrazine, 5-Ethyl-2,3-Dimethylpyrazine, Ethyl 2,4-Dioxohexanoate, Ethylenediamine, Ethylenediaminetetraacetic Acid Disodium Salt, Ethylene Dichloride, Ethylene Glycol Distearate, Ethylene Glycol Monobutyl Ether, Ethylene Glycol Monoethyl Ether, Ethylene Glycol Monophenyl Ether, Ethylene Oxide, Ethylene Oxide Polymer, Ethylene Oxide Polymer, Alkyl Adduct, Ethylene Oxide Polymer, Alkyl Adduct, Phosphate Ester, Ethylene Oxide/Propylene Oxide Copolymer (Avg M W 9,760-13,200), Ethylene Oxide/Propylene Oxide Copolymer (Min Avg M W 1,900), Ethylene Oxide/Propylene Oxide Copolymer (Avg M W 14,000), Ethylene Oxide/Propylene Oxide Copolymer, Ethylene Oxide/Propylene Oxide Copolymer (Avg M W 3,500-4,125), Ethylene Oxide/Propylene Oxide Copolymer, Alkyl Adduct, Ethylene Oxide/Propylene Oxide Copolymer, Alkyl Adduct, Phosphate Ester, Ethyl Esters Of Fatty Acids (Edible), Ethyl N-Ethylanthranilate, Ethyl 2-Ethylbutyrate, Ethyl 2-Ethylhexanoate, Ethyl Alpha-Ethyl-Beta-Methyl -Beta-Phenylglycidate, Ethyl 2-Ethyl-3-Phenylpropanoate, Ethyl 3-(Ethylthio)Butyrate, Ethyl Formate, 2-Ethylfuran, Ethyl 2-Furanpropionate, Ethyl Furfuryl Ether, Ethyl 3-(Furfurylthio) Propionate, Ethyl 3-(2-Furyl)Acrylate, 4-Ethylguaiacol, Ethyl Heptanoate, 2-Ethyl-2-Heptenal, Ethyl 4-Heptenoate, Cis-, 2-Ethythexanethiol, Ethyl Hexanoate, 2-Ethyl-1-Hexanol, 2-Ethyl-2-Hexenal, Ethyl Cis-3-Hexenoate, Ethyl 5-Hexenoate, Ethyl 3-Hexenoate, Ethyl 2-Hexenoate, 2-Ethylhexyl Benzoate, 2-Ethylhexyl 3-Mercaptopropionate, 1-Ethylhexyl Tiglate, Ethyl 3-Hydroxybutyrate, 3-Ethyl-2-Hydroxy-2-Cyclopenten-1-One, Ethyl 5-Hydroxydecanoate, Ethyl 2-Hydroxyethyl Sulfide, Ethyl 3-Hydroxyhexanoate, (+/−)-Ethyl 2-Hydroxy-2-Methylbutyrate, 3-Ethyl-2-Hydroxy-4-Methylcyclopent-2-En-1-One, 5-Ethyl-2-Hydroxy-3-Methylcyclopent-2-En-1-One, 5-Ethyl-3-Hydroxy-4-Methyl-2(5h)-Furanone, 2-Ethyl-4-Hydroxy-5-Methyl-3(2h)-Furanone, (+/−)-Ethyl 2-Hydroxy-3-Methylvalerate, Ethyl 3-Hydroxyoctanoate, Ethyl 5-Hydroxyoctanoate, Ethyl 2-Hydroxy-3-Phenylpropionate, Ethyl Isobutyrate, N-Ethyl-2-Isopropyl-5-Methylcyclohexane Carboxamide, Ethyl Isothiocyanate, Ethyl Isovalerate, Ethyl Lactate, Ethyl Laurate, Ethyl Levulinate, Ethyl Levulinate Propyleneglycol Ketal, Ethyl ILinalyl Ether, Ethyl Maltol, Ethyl Maltol Isobutyrate, Ethyl 3-Mercaptobutyrate, (+−)-Ethyl 3-Mercapto-2-Methylbutanoate, Ethyl 2-Mercapto-2-Methylpropionate, Ethyl 3-Mercaptopropionate, Ethyl 2-Mercaptopropionate, Ethyl N-Methylanthranilate, Ethyl 2-Methylbutyrate, Ethyl Methyl Disulfide, Ethyl 2-Methyl-3,4-Pentadienoate, Ethyl 4-Methylpentanoate, Ethyl 3-Methylpentanoate, Ethyl 2-Methylpentanoate, Ethyl 2-Methyl-3-Pentenoate, Ethyl 2-Methyl-4-Pentenoate, 3-Ethyl-2-Methylpyrazine, 2-Ethyl-6-Methylpyrazine, 2-Ethyl-5-Methylpyrazine, 5-Ethyl-2-Methylpyridine, 5-Ethyl-2-Methylthiazole, 2-Ethyl-4-Methylthiazole, Ethyl 4-(Methylthio) Butyrate, Ethyl 3-(Methylthio)-Cis-2-Propenoate, Ethyl 3-(Methylthio)-2-Propenoate, Ethyl 3-Methylthiopropionate, 2-Ethyl-3-Methylthiopyrazine, Ethyl 3-(Methylthio)-Trans-2-Propenoate, Ethyl 5-(Methylthio)Valerate, Ethyl Methyl-P-Tolylglycidate, 2-Ethyl-(3 Or 5 Or 6)-Mop (85%) And 2-Methyl-(3 Or 5 Or 6)-Mop (13%), Ethyl Myristate, Ethyl Nitrite, N-Ethyl Trans-2-Cis-6-Nonadienamide, Ethyl Nonanoate, Ethyl 2-Nommoate, Ethyl Octadecanoate, Ethyl Cis-4,7-Octadienoate, (+/−)-4-Ethyloctanal, Ethyl Octanoate, 4-Ethyloctanoic Acid, Ethyl Cis-4-Octenoate, Ethyl 3-Octenoate, Ethyl Trans-2-Octenoate, Ethyl Oleate, 2-Ethyl-3,(5 Or 6)-Dimethylpyrazine, Ethyl 5-Oxodecanoate, Ethyl 3-Oxohexanoate, Ethyl Palmitate, Ethyl 4-Pentenoate, P-Ethylphenol, Ethyl Phenylacetate, Ethyl 4-Phenylbutyrate, Ethyl 3-Phenylglycidate, Ethyl 3-Phenylpropionate, Ethyl Propionate, Ethyl 2-(Methyidithio)Propionate, Ethyl Propyl Disulfide, Ethyl Propyl Trisulfide, 2-Ethylpyrazine, 3-Ethylpyridine, 1-Ethyl-2-Pyrrolecarboxaldehyde, Ethyl Pyruvate, Ethyl Salicylate, Ethyl Sorbate, Ethyl Thioacetate, (+/−)-3-(Ethylthio)Butanol, 2-Ethylthiophenol, Ethyl Tiglate, Ethyl Trans-2-Hexenoate, Ethyl Trans-2-Methyl-2-Pentenoate, 2-Ethyl-1,3,3-Trimethyl-2-Norbornanol, Ethyl Undecanoate, Ethyl 10-Unddecenoate, Ethyl Valerate, Ethyl Vanillin, Ethyl Vanillin Beta-D-Glucopyranoside, Ethyl Vanillin Isobutyrate, Ethyl Vanillin Propylene Glycol Acetal, Eucalyptol, Eucalyptus, Oil (Eucalyptus Globulus Labille), Eugenol, Eugenyl Acetate, Eugenyl Benzoate, Eugenyl Formate, Eugenyl Isovalerate, Eugenyl Methyl Ether, (2e,6z,8e)-N-(2-Methylpropyl)-2,6,8-Decatrienamide, Famesal, Farnesene, Farnesol, Fatty Acids, Fatty Alcohols, Synthetic, Fd&C Blue No. 2, Fd&C Blue No. 1, Fd&C Blue No. 2, Aluminum Lake, Fd&C Blue No. 1, Aluminum Lake, Fd&.0 Blue No. 2, Calcium Lake, Fd&C Blue No. 1, Calcium Lake, Fd&C Green No. 3, H&C Green No. 3, Aluminum Lake, Fd&C Green No. 3, Calcium Lake, Fd&C Red No. 40, Fd&C Red No. 3, Fd&C Red No, 40, Aluminum Lake, Fd&C Red No. 3, Fd&C Red No. 40, Calcium Lake, Fd&C Red No. 3, Fd&C Yellow No. 6, Fd&C Yellow No. 5, Fd&C Yellow No. 5, Aluminum Lake, Fd&C Yellow No. 6, Aluminum Lake, Fd&.0 Yellow No. 5, Calcium Lake, Fd&C Yellow No. 6, Calcium Lake, D-Fenchone, Fenchyl Alcohol, Fennel, Common (Foeniculum Vulgare Mill.), Fennel, Sweet (Foeniculum Vulgare Mill. Var. Dulce (D.C.) Alef.), Fennel, Sweet, Oil (Foeniculum Vulgare Var. Dulce (D.C.) Alef.), Fenugreek (Trigonella Foenum-Graeeum L.), Fenugreek, Extract (Trigonella Foenum-Graecum L), Fenugreek, Oleoresin (Trigonella Foenum-Graecum L.), Ferric Ammonium Citrate, Brown, Ferric Chloride, Ferric Citrate, Ferric Oxide, Ferric Peptonate, Ferric Phosphate, Ferric Pyrophosphate, Ferric Sodium Pyrophosphate, Ferric Sulfate, Ferrocyanide Salts, Ferrous Ascorbate, Ferrous Carbonate, Ferrous Citrate, Ferrous Fumarate, Ferrous Gluconate, Ferrous L-Lactate, Ferrous Lactate, Ferrous Peptonate, Ferrous Sulfate, Ficin, Fir (Pine) Needles And Twigs (Abies Sibirica Ledeb.), Fir Needles And Twigs, Oil (Abies Spp.), Fish Oil (Hydrogenated), Fish Protein Concentrate, Whole, Fish Protein Isolate, Folic Acid, Formaldehyde, Formic Acid, 2-Formyl-6,6-Dimethylbicyclo(3.1.1)Hept-2-Ene, 4-Formyl-2-Methoxyphenyl 2-Hydroxypropanoate, Fruit Juice, Fullers Earth, Fumaric Acid, Fungal Hemicellulase, Fungal Pectinase, 2-Furanmethanethiol Formate, 4((Furanmethyl)Thio)-2-Pentanone, Furcelleran, Furcelleran, Ammonium Salt Of, Furcelleran And Salts Of Furcelleran, Furcelleran, Calcium Salt Of, Furcelleran, Potassium Salt Of, Furcelleran, Sodium Salt Of, Furfural, Furfural Propyleneglycol Acetal, Furfuryl Acetate, Furfuryl Alcohol, Furfuryl Butyrate, Furfuryl Decanoate, Furfuryl Formate, 2-Furfurylidenebutyraldehyde, Furfuryl Isopropyl Sulfide, Furfuryl Mercaptan, Furfuryl 3-Methylbutanoate, Furfuryl Methyl Ether, Furfuryl 2-Methyl-3-furyl Disulfide, Furfuryl Methyl Sulfide, Alpha-Furfuryl Octanoate, Alpha-Furfuryl Pentanoate, Furfuryl Propionate, Furfuryl Propyl Disulfide, N-Furfurylpyrrole, Furfuryl Thioacetate, 1-(2-Furfurylthio)Propanone, Furfuryl Thiopropionate, 3-(2-Furoylthio)-2,5-Dimethylfuran, 1-(2-Furyl)-1,3-Butanedione, 1-(2-Furyl)Butan-3-One, 4-(2-Furyl)-3-Buten-2-One, 2-Furyl Methyl Ketone, Fusel Oil, Refined, Alpha-Galactosidase From Morteirella Vinaceae Raffinoseutilizer, Galanga, Greater (Alpinia Galanga Wildd), Galangal Root (Alpinia Spp.), Galangal Root, Extract (Alpinia Spp.), Galangal Root, Oil (Alpinia Spp.), Galbanum, Oil (Ferula Spp.), Galbanum, Resin (Ferula Spp.), Gambir (Uncaria Gambir Roxb.), Gardenia Gummifera Distillate, Garlic, Garlic Extract, Garlic Oil (Allium Sativum L.), Dehydrated Garlic, Garlic Powder, Gelatin, Gellan Gum, Genet Absolute (Spartium Junceum L.), Genet Extract (Spartium Junceum L.), Gentian Root Extract (Gentiana Lutea L.), Gentian, Stemless (Gentiana Acaulis L.), Geranic Acid, Geraniol, Geranium (Pelargonium Spp.), Geranium, East Indian, Extract (Cymbopogon Martini Stapf.), Geranium, East Indian, Oil (Cymbopogon Martini Stapf.), Geranium Extract (pelargonium Spp.), Geranium, Oil (Pelargonium Spp.), Geranium, Rose, Oil (Pelargonium Graveolens L'her.), Geranyl Acetate, Geranyl Acetoacetate, Geranyl Acetone, Geranyl Benzoate, Geranyl Butyrate, Geranyl Formate, Geranyl Hexanoate, Geranyl Isobutyrate, Geranyl Isovalerate, Geranyl 2-Methylbutyrate, Geranyl Phenylacetate, Geranyl Propionate, Geranyl Tiglate, Geranyl Valerate, Germander, Chamaedrys (Teucrium Chamaedrys L.), Germander, Chamaedrys, Extract (Teucrium Chamaedrys L.), Germander, Chamaedrys, Extract Solid (Teucrium Chamaedrys L.), Germander, Golden (Teucrium Polium L.), Ghatti, Gum (Anogeissus Latifolia Wall.), Gibberellic Acid & Potassium Gibberellate, Ginger (Zingiber Officinale Rosc.), Ginger, Extract (Zingiber Officinale Rosc.), Ginger, Oil (Zingiber Officinale Rosc.), Ginger, Oleoresin (Zingiber Officinale Rosc.), D-Gluconic Acid, Glucono-Delta Lactone, N-Gluconyl Ethanolamine, N-Gluconyl Ethanolamine Phosphate, Glucose Isomerase From Bacillus Coagulans, Glucose Isomerase From Immobilized Arthrobacter Globiformis, Glucose Isomerase From Streptomyces Olivaceus, Glucose Isomerase From Streptomyces Olivochromogenes, Glucose Isomerase From Streptomyces Rubiginosus, Glucose Oxidase Catalase Preparation, Glucose Oxidase From Aspergillus Niger, Glucose Oxidase From penicillium Notatum, Glucose Pentaacetate, Glucosidase From Aspergillus Flavus, Glucosidase From Aspergillus Niger, Glucosidase From Aspergillus Oryzae, L-Glutamic Acid, Glutamic Acid Hydrochloride, L-Glutamine, Gamma-Glutamyl-Valyl-Glycine, Glutaraldehyde, Gluten, Gum, Glycerin, Glycerin, Synthetic, Glycerol Tributyrate, Glyceryl Behenate, Glyceryl 5-Hydroxydecanoate, Glyceryl 5-Hydroxydodecanoate, Glyceryl-Lacto Esters Of Fatty Acids, Glyceryl Lactooleate, Glyceryl Lactopalmitate, Glyceryl Monooleate, Glyceryl Monostearate, Glyceryl Palmitostearate, Glyceryl Tribenzoate, Glyceryl Tripropanoate, Glyceryl Tristearate, Glycine, Glycocholic Acid, Glycyrrhizin, Ammoniated (Glycyrrhiza Spp.), Grains Of Paradise (Aframomum Melegueta (Rosc.) K. Schum.), Grape Color Extract, Grape Essence, Natural, Grapefruit Essence, Natural, Grapefruit, Extract, Grapefruit, Juice, Grapefruit, Oil (Citrus Paradisi Macf.), Grapefruit Oil, Conc., Grapefruit, Oil, Terpeneless (Citrus Paradisi), Grape Seed Extract, Grape Skin Extract, Ground Limestone, Guaiac Gum (Guaiacum Spp.), Guaiac Gum, Extract (Guaiacum Spp.), Guaiacol, Guaiacol Butyrate, Guaiacol Propionate, Guaiac Wood, Extract (Guaiacum Spp.), Guaiac Wood, Oil (Guaiacum Spp.), Guaiacyl Acetate, Guaiacyi Isobutyrate, Guaiacyl Phenylacetate, Guaiene, Guaiol Acetate, Guarana, Gum (Paullinia Cupana Hbk), Guarana Seed, Extract, Guar, Gum (Cyamopsis Tetragonolobus (L.)), Guava (Psidium Spp.), Guava Extract, Gum Arabic, Hydrogen Octenylbutane Dioate, Gutta Hang Kang (Pataquium Leiocarpum Boerl. And P. Oblongifolium Burck.), Haematococcus Algae Meal, Haw Bark, Black, Extract (Viburnum Prunifolium L.), Heliopsis Longipes Extract, Helium, Hemlock (Tsuga Spp.), Hemlock Needles And Twigs, Oil (Tsuga Spp.), 2,4-Heptadienal Trans-2-Trans-4-Heptadien-1-O1, Gamma-Heptalactone, Heptanal, Heptanal Dimethyl Acetal, Heptanal Glyceryl Acetal (Mixed 1,2 And 1,3 Acetals), Heptanal Propyleneglycol Acetal, 2,3-Heptanedione, 2-Heptanethiol, Heptane-1-Thiol, Heptanoic Acid, 2-Heptanol, 3-Heptanol, 2-Heptanone, 3-Heptanone, 4-Heptanone, N-(Heptan-4-Y1)Benzo[D][1,3]Dioxole-5-Carboxamide, 2-Heptenal, 4-Heptanal Diethyl Acetal, (E)-2-Heptenoic Acid, (+/−)-1-Hepten-3-O1, (Z)-4-Hepten-1-O1, 3-Hepten-2-One, 2-Hepten-4-One, Hept-Trans-2-En-1-Y1 Acetate, Hept-2-En-1-Y1 Isovalerate, Trans-3-Heptenyl 2-Methylpropanoate, 3-Heptyl Acetate, Heptyl Acetate, Heptyl Alcohol, Heptyl Butyrate, 2-Heptyl Butyrate, Heptyl Cinnamate, Cis- And Trans-2-Heptylcyclopropanecarboxylic Acid, 3-Heptyldihydro-5-Methyl-2(3h)-Furanone, Heptyl Formate, 2-Heptylfuran, Heptyl Heptanoate, Heptyl Isobutyrate, Heptyl Octanoate, Heptylparaben, Hesperidin, Delta-Hexadecalactone, Omega-6-Hexadecenlactone, Hexadecyl Lactate, Trans, Trans-2,4-Hexadienal, 2,4-Hexadien-1-O1, 2,4-Hexadienyl Acetate, 2,4-Hexadienyl Butyrate, 2,4-Hexadienyl Isobutyrate, 2,4-Hexadienyl Propionate, Delta-Hexalactone, Gamma-Hexalactone, Hexanal, Hexanal Butane-2,3-Diol Acetal, Hexanal Dihexyl Acetal, Hexanal Hexyl Isoamyl Acetal, Hexanal Octane-1,3-Diol Acetal, Hexane, 2,3-Hexanedione, 3,4-Hexanedione, 1,6-Hexanedithiol, 1-Hexanethiol, Hexanoic Acid, 3-Hexanol, 3-Hexanone, Cis-3-Hexenal, Trans-4-Hexenal, 3-Hexenal, Cis-4-Elexenal, Trans-3-Hexenal, 2-Hexenal, (+/−)-Trans- And Cis-2-Hexenal Glyceryl Acetal, (E)-2-Hexenat Diethyl Acetal, 4-Hexene-3-One, (Z)-3-Hexenyl (E)-2-Hexenoate, Trans-2-Hexenoic Acid, 3-Hexenoic Acid, 1-Hexen-3-O1, 2-Hexen-1-O1, 5-Hexenol, (Z)-2-Hexen-1-O1, (Z)-3-Hexenyl Anthranilate, Cis-3-Hexenyl Benzoate, (E)-2-Hexenyl Butyrate, 3-Hexenyl Crotonate, Cis-, (E)-2-Hexenyl Formate, 2-Hexenyl Hexanoate, Trans-, (Z)-3-Hexeniyl Isobutyrate, 5-Hexenyl Isothiocyanate, (E)-2-Hexenyl Isovalerate, 3-Hexenyl Isovalerate, Cis-3-Hexenyl Lactate, 3-Hexenyl 2-Methylbutyrate, 2-Hexenyl Octanoate, 3-Hexenyl Phenylacetate, (E)-2-Hexenyl Propionate, Cis-3 & Trans-2-Hexenyl Propionate, (Z)-3-Hexenyl Propionate, (Z)-3-Hexenyl Pyruvate, (Z)-3-Hexenyl Valerate, (E)-2-Hexenyl Valerate, Hexyl Acetate, 2-Hexyl-4-Acetoxytetrahydrofuran, Hexyl Alcohol, Hexylamine, Hexyl Benzoate, N-hexyl 2-Butenoate, Hexyl Butyrate, Alpha-Hexylcinnamaldehyde, Hexyl Decanoate, 2-Hexyl-4,5-Dimethyl-1,3-Dioxolane, Hexyl Formate, Hexyl 2-Furoate, Hexyl Heptanoate, Hexyl Hexanoate, Hexyl Trans-2-Hexenoate, 2-Hexylidene Cyclopentanone, 2-Hexylidenehexanal, Hexyl Isobutyrate, Hexyl Isothiocyanate, Hexyl Isovalerate, 2-Hexyl-5 Or 6-Keto-1,4-Dioxane, Hexyl 3-Mercaptobutanoate, Hexyl 2-Methylbutyrate, Hexyl 2-Methyl-3 (Or 4)-Pentenoate, Hexyl Nonanoate, Hexyl Octanoate, Hexyl Phenylacetate, Hexyl Propionate, 2-Hexylthiophene, Hickory Bark, Extract (Carya Spp.), Hickory Smoke Dist., High Fructose Corn Syrup, L-Histidine, (−)-Homoeriodictyol, Sodium Salt, Honeysuckle Extract, Hops, Extract (Humulus Lupulus L.), Hops Extract, Modified, Hops, Extract Solid (Humulus Lupulus L.), Hops, Oil (Humulus Lupulus L.), Horehound Extract (Marrubium Vulgare L.), Horehound (Marrubium Vulgare L.), Horehound Solid, Extract, Horsemint Leaves, Extract (Monarda Spp.), Horseradish (Armoracia Lapathifolia Gilib.), Horseradish Oil, Hyacinth, Absolute (Hyacinthus Orientalis L.), Hyacinth Flowers (Hyacinthus Orientalis L.), Hydratropic Aldehyde Propylene Glycol Acetal, Hydrazine, Hydrochloric Acid, Hydrogen Peroxide, Hydrogen Sulfide, Hydroquinone Monoethyl Ether, Hydroxyacetone, 4-Hydroxyacetophenone, 2-Hydroxyacetophenone, 4-Hydroxybenzaldehyde, 4-Hydroxybenzoic Acid, 3-Hydroxybenzoic Acid, 4-Hydroxybenzyl Alcohol, 4-Hydroxybutanoic Acid Lactone, 1-Hydroxy-2-Butanone, 4-Hydroxy-2-Butenoic Acid Gamma-Lactone, 6-Hydroxycarvone, Hydroxy citronellal, Hydroxycitronellal Diethyl Acetal, Hydroxycitronellal Dimethyl Acetal, Hydroxycitronellal Propyleneglycol Acetal, Hydroxycitronellol, 2-Hydroxy-2-Cyclohexen-1-One, 5-Hydroxy-2,4-Decadienoic Acid Delta-Lactone, 5-Hydroxy-2-Decenoic Acid Delta-Lactone, 5-Hydroxy-7-Decenoic Acid Delta-Lactone, 6-Hydroxydihydrotheaspirane, 4-Hydroxy-3,5-Dimethoxybenzaldehyde, 4-Hydroxy-2,5-Dimethyl-3(2h)-Furanone, 4-Hydroxy-2,3-Dimethyl-2,4-Nonadienoic Acid Gamma Lactone, 6-Hydroxy-3,7-Dimethyloctanoic Acid Lactone, (Z)-4-Hydroxy-6-Dodecenoic Acid Lactone, 5-Hydroxy-2-Dodecenoic Acid Lactone, 1-Hydroxyethylidene-1,1-Diphosphonic Acid, 4-Hydroxy-4-(3-Hydroxy-1-Butenyl)-3,5,5-Tritnethyl-2-Cyclohexen-1-One, Hydroxy(4-Hydroxy-3-Methoxyphenyl)Acetic Acid, 1-(2-Hydroxy-4-Isobutoxyphenyl)-3-(Pyridin-2-Y1)Propan-1-One, Hydroxylated Lecithin, 2-Hydroxy-4-Methoxybenzaldehyde, N-(4-Hydroxy-3-Methoxybenzyl)-8-Methyl-6-Nonenamide, 1-(4-Hydroxy-3-Methoxyphenyl)Decan-3-One, 1-(2-Hydroxy-4-Methoxyphenyl)-3-(Pyridin-2-Y1)Propan-1-One, 2-Hydroxy-5-Methylacetophenone, 2-Hydroxy-4-Methylbenzaldehyde, 2-(2-Hydroxy-4-Methyl-3-Cyclohexenyl)Propionic Acid Gamma-Lactone, 4-Hydroxy-4-Methyl-7-Cis-Decanoic Acid Gamma Lactone, 2-Hydroxymethyl-6,6-Dimethylbicyclo(3.1.1)Hept-2-Enyl Formate, 4-Hydroxymethyl-2,6-Di-Tertbutylphenol, 10-Hydroxymethylene-2-Pinene, 4-Hydroxy-5-Methyl-3(2h)-Furanone, 3-(Hydroxymethyl)-2-Heptanone, 5-Hydroxy-4-Methylhexanoic Acid Delta-Lactone, 3-Hydroxy-5-Methyl-2-Hexanone, 2-Hydroxy-5-Methyl-3-Hexanone, 4-Hydroxy-4-Methyl-5-Hexenoic Acid Gamma Lactone, (?)-3-Hydroxy-3-Methyl-2,4-Nonanedione, 4-Hydroxy-3-Methyloctanoic Acid Lactone, 1-Hydroxy-4-Methyl-2-Pentanone, 1-(3-Hydroxy-5-Methyl-2-Thienyl)Ethanone, Hydroxynonanoic Acid, Delta-Lactone, 3-Hydroxy-2-Octanone, 5-Hydroxy-4-Octanone, 3-Hydroxy-2-Oxopropionic Acid, 3-Hydroxy-2-Pentanone, 4-Hydroxy-3-Pentenoic Acid Lactone, 4-(P-Hydroxyphenyl)-2-Butanone, 3-Hydroxy-4-Phenylbutan-2-One, 2-(2-Hydroxyphenyl) Cyclopropanecarboxylic Acid Delta Lactone, 1-(2-Hydroxyphenyl)-3-(Pyridin-4-Y1)Propan-1-One, (+/−)-2-Hydroxypiperitone, L-Hydroxyproline, Hydroxypropyl Cellulose, Hydroxypropyl Methylcellulose, 2-Hydroxy-3,5,5-Trimethyl-2-Cyclohexenone , 5-Hydroxyundecanoic Acid Lactone, 5-Hydroxy-8-Undecenoic Acid Delta-Lactone, Hyssop, Extract (Hyssopus Officinalis L.), Hyssop (Hyssopus Officinalis L.), Hyssop, Oil (Hyssopus Officinalis L.), Iceland Moss (Cetraria Islanilica Ach,), Immortelle, Absolute (Helichrysum Angustifolium Dc), Immortelle, Extract (Helichrysum Angustifolium Dc.), Imperatoria (Peucedanum Ostruthium (L.) Koch (Imperatoria Ostruthium L.)), Indole, Inositol, Insoluble Glucose Isomerase Enzyme Preparations, Invertase From Saccharomyces Cerevisiae, Invert Sugar, Invert Sugar Syrup, Alpha-Ionene, Ion Exchange Membranes, Ion Exchange Resin, Beta-Ionol, Alpha-Ionol, Beta-Ionone, Gamma-Ionone, Alpha-Ionone, Beta-Ionone. Epoxide, Beta-Ionyl Acetate, Iron Ammonium Citrate, Iron Caprylate, Iron-Choline Citrate Complex, Iron Citrate, Alpha-Irone, Iron, Elemental, Iron Linoleate, Iron Naphthenate, Iron Oxide, Iron Peptonate, Iron Polyvinylpyrrolidone, Iron Tallate, Isoambrettolide, Isoamyl Acetate, Isoamyl Acetoacetate, Isoamyl Alcohol, Isoamyl Benzoate, Isoamyl Butyrate, Isoamyl Cinnamate, Isoamyl Formate, Isoamyl 4-(2-Furan)Butyrate, Isoamyl 3-(2-Furan)Propionate, Isoamyl Hexanoate, Isoamyl Isobutyrate, Isoamyl Isothiocyanate, Isoamyl Isovalerate, Isoamyl Laurate, Isoamyl Levulinate, Isoamyl 2-Methylbutyrate, Isoamyl Nonanoate, Isoamyl Octanoate, Isoamyl Phenethyl Ether, Isoamyl Phenylacetate, Isoamyl Propionate, Isoamyl Pyruvate, Isoamyl Salicylate, Isoborneol, Isobornyl Acetate, Isobornyl Formate, Isbornyl Isobutyrate, Isobornyl Isovalerate, Isobornyl 2-Methylbutyrate, Isobornyl Propionate, Isobutane, Isobutyl Acetate, isobutyl Acetoacetate, Isobutyl Alcohol, Isobutylamine, Isobutyl Angelate, Isobutyl Anthranilate, Isobutyl Benzoate, Isobutyl 2-Butenoate, Isobutyl Butyrate, Isobutyl Cinnamate, N-Isobutyldeca-Trans-2-Trans-4-Dienamide, 2(4)-Isobutyl-4(2),6-Dimethyldihydro-4h-1,3,5-Dithiazine, 2-Isobutyl-4,5-Dimethyloxazole, Isobutylene-Isoprene Copolymer, Isobutyl Formate, Isobutyl 2-Furanpropionate, Isobutyl Heptanoate, Isobutyl Hexanoate, Isobutyl Isobutyrate, Isobutyl Isothiocyanate, 2-Isobutyl-3-Methoxypyrazine, Isobutyl N-Methylanthranilate, 2-Isobutyl-3-Methylpyrazine, (+/−)-Isobutyl 3-Methylthiobutyrate, Isobutyl Phenylacetate, Isobutyl Propionate, Isobutyl Salicylate, 2-Isobutylthiazole, Isobutyl 10-Undecenoate, Isobutyraldehyde, Isobutyric Acid, Isocyclocitral, Isoeugenol, Isoeugenyl Acetate, Isoeugenyl Benzyl Ether, Isoeugenyl Ethyl Ether, Isoeugenyl Formate, Isoeugenyl Methyl Ether, Isoeugenyl Phenylacetate, Isojasmone, L-Isoleucine, Alpha-Isomethylionone, Beta-Isomethylionone, Alpha-Isomethylionyl Acetate, Isoparaffinic Petroleum Hydrocarbons, Synthetic, Isopentamine, Isopentylideneisopentylamine, Isophorone, Isopropenyl Acetate, 5-Isopropenyl-2-Methyl-2-Vinyltetrahydrofuran, 3-Isopropenyl-6-Oxoheptanoic Acid, 3-Isopropenylpentanedioic Isopropenylpentanedioic Acid, Isopropenylpyrazine, Isopropyl acetate, P-Isopropylacetophenone, Isopropyl Alcohol, Isopropylamine, Isopropyl Benzoate, P-Isopropylbenzyl Alcohol, Isopropyl Butyrate, Isopropyl Cinnamate, Isopropyl Citrate, Isopropyl-2Cyclohexenone, 5-Isopropyl-2,6-Diethyl-2-Methyltetrahydro-2h-Pyran, 2(4)-Isopropyl-4(2),6-Dimethyldihydro-4h-1,3,5-Dithiazine, Isopropyl Formate, Isopropyl Hexanoate, Isopropylideneglyceryl 5-Hydroxydecanoate, Isopropyl Isobutyrate, Isopropyl Isothiocyanate, Isopropyl Isovalerate, S-Isopropyl 3-Methylbut-2Enethioate, Isopropyl 2-Methylbutyrate, 2-Isopropyl-5-Methyl-2-Hexenal, (+/−)-[R-(E)]-5-Isopropyl-8-Methylnona-6,8-Dien-2-One, 2-Isopropyl-4-Met hylthiazole, Isopropyl Myristate, Isopropyl Palmitate, 2-Isopropylphenol, P-Isopropylphenyl acetaldehyde , Isopropyl Phenylacetate, 3-(P-Isopropylphenyl)Propionaldehyde, Isopropyl Propionate, 2-Isopropylpyrazine, Isopropyl Tiglate, 2-Isopropyl-N,2,3-Trimethylbutyramide, Isopulegol, Isopulegone, Isopulegyl Acetate, Isoquercitrin, Enzymatically Modified, Isoquinoline, Isovaleraldehyde, Diethyl Acetal, Isovaleraldehyde Glyceryl Acetal, Isovaleric acid, Iva (Achillea Moschata Jacq.), Iva, Extract (Achillea Moschata Jacq.), Jambu Oleroesin, Japan Wax, Jasmine, Absolute (Jasminum Spp.), Jasmine, Concrete (Jasminum Spp.), Jasmine, Oil (Jasminum Grandiflorum L.), Jasmine, Spiritus (Jasminum Grandiflorum L.), Jasmone, Cis-, Jelutong (Dyera Costulata Hook F. And D. Lowii Hook, F.) Junipter (Berries) (Juniperus Communis L.), Juniper, Extract (Juniperus Communis L. ), Juniper Oil (Juniperus Communis L.), Karaya, Gum (Sterculia Urens Roxb.), Kelp, 2-Keto-4-Butanethiol, Alpha-Ketobutyric Acid, Kola Nut, Extract (Cola Acuminata Schott Et Endl.), Labdanum, Absolut e (Cistus Spp.), Labdanum, Oil (Cistus Spp.), Labdanum, Oleresin (Cistus Spp.), Lactalbumin, Lactalbumin Phosphate, Lactase From Saccharomyces Fragilis, Lactase From Saccharomyces (Kluyveromyces) Lactis, Lactase Preparation, Candida Pseudotropicalis, Lactic Acid, Lactose, Lactose, Hydrolyzed, N-Lactoyl Ethanolamine, N-Lactoyl ethanolamine Phosphate, Lactylated Fatty Acid Esters of Glycerol And Propylene Glycol, Lactylic Esters Of Fatty Acids, Lanolin, Lard, Lard Oil, Laurel Berries (Laurus Nobilis L.), Lauric Acid, Lauric Aldehyde, Lauroyl Diethanolamide, Laurel Acetate, Lauryl Alcohol, Lavandin Absolute, Lavandin, Concrete, Lavandin, Oil, Lavender, Absolute (Lavandula Officinalis Chaix), Lavender, Concrete (Lavandula Officinalis Chaix), Lavender (Lavandula Officinalis Chaix), Lavender, Oil (Lavandula Officnialis Chaix), Lavender, Spike (Lavandula Latifolia Bill.), Lavender, Spike, Oil (Lavandula Spp.), Leche Caspi (Couma Macrocarpa Barb. Rodr.), Leche De Vaca (Brosimum Utile (H.B.K.) Pittier, And Poulsenia Spp.), Lecithin, Lecithin, Benzoyl Peroxide Modified, Lecithin, Enzyme-Modified, Lecithin, Hydrogen Peroxide Modified, Lecithin (Vegetable) Leek Oil, Lemon Essence, Lemon, Extract (Citrus Limon (L.) Burm. F.), Lemon Grass, Oil (Cymbopogon Citratus Dc. And Cymbopogon Flexuosusstapf), Lemon, Juice, Lemon, Oil (Citrus Limon (L.) Burm. F.), Lemon, Oil, Terpeneless (Citrus Limon (L.) Burm. F.), Lemon Peel Extract, Lemon Peel Granules, Lemon Terpenes, Lemon-Verbena (Lippia Citriodora Hbk. ), Lemon Verbena, Oil (Lippia Citriodora), Lepidine, L-Leucine, Levulinic Acid, Levulose, L-Fenchone, Licorice Extract (Glycyrrhiza Spp.), Licorice Extract Powder (Glycyrrhiza Spp.), Licorice (Glycyrrhiza Spp.), Lignin, Lignin Sodium Sulfonate, Lignosulfonic Acid, Lime, Essence, Lime, Juice, Lime Juice, Dehydrated, Lime Oil, Distilled, Lime Oil, Expressed, Lime, Oil, Terpeneless (Citrus Aurantifolia (Christman) Swingle), L-Limonene D-Limonene, D1-Limonene, Linaloe Wood, Oil (Bursera Delpechiana Poiss. And Other Bursera Spp.), Linalool, Linalool Oxide, Linalool Oxide Pyranoid, Linalyl Acetate, Linalyl Anthranilate, Linalyl Benzoate, Linalyl Butyrate, Linalyl Cinnamate, Linalyl Formate, Linalyl Hexanoate, Linalyl Isobutyrate, Linalyl Isovalerate, Linalyl Octanoate, Linalyl Phenylacetate, Linalyl Propionate, Linden Flowers, Extract (Tilia Spp.), Linden Flowers (Tilia Glabra Vent.), Linden Leaves (Tilia Spp.), Linoleic Acid, Lipase, Lipase From Animal Tissue, Lipase From Aspergillus Niger, Lipase From Aspergillus Oryzae, Lipase from Rhizopus Niveus, Listeria-Specific Bacteriophage Preparation, Litsea Cubeba Berry Oil, L-Mentyl Butyrate, Locust (Carob) Bean Gum, Lovage, Extract (Levisticum Officinale Koch). Lovage (Levisticum Officinale Koch), Lovage, Oil (Levisticum Officinale Koch), L-8P-Menthene-1,2-Epoxide, Lungmoss (Sticta Pulmonacea Ach.), Luo Han Fruit Concentrate, Lupulin (Humulus Lupulus L.) L-Lysine, Mace (Myristica Fragrans Houtt.), Mace, Oil (Myristica Fragrans Houtt.), Mace, Oleoresin (Myristica Fragrans Houtt.), Magnesium caprate, Magnesium Caprylate, Magnesium Carbonate, Magnesium chloride, Magnesium Fumarate, Magnesium Gluconate, Magnesium Glycerophosphage, Magnesium Hydroxide, Magnesium Laurate, Magnesium Myristate, Magnesium Oleate, Magnesium Oxide, Magnesium Palmitate, Magnesium Phosphate, Dibasic, Magnesium Phosphate, Tribasic, Magnesium Salts Of Fatty Acids, Magnesium Silicate, Magnesium Stearate, Magnesium Sulfate, Magnolol, Maidenhair Fern (Adiantum Capills-Veneris L.), L-Malic Acid, Malic Acid, Malt, Maltodextrin, Maltol, Maltol Propionate, Maltose, Malt Syrup (Malt Extract), Maltyl Isobutyrate, Mandarin, Oil (Citrus Reticulata Blanco), Manganese Chloride, Manganese Citrate, Manganese Gluconate, Manganese Glycerophosphate, Manganese Hypophosphite, Manganese Sulfate, Manganous Oxide, Mannitol, Marigold, Pot (Calendula Officinalis L.), Marjoram, Oleoresin (Marjorana Hortensis (Origanum Marjorana L.)), Marjoram, Pot (Marjorana Onites (L.) Benth. (Origanum Vulgare L.)), Marjoram Seed (Marjorana Hortensis Moench (Origanum Marjoana L.)), Marjoram, Sweet (Marjorana Hortensis Moench (Origanum Jarjorana L.)), Marjoram, Sweet, Oil (Marjorana Hortensis Moench (Origanum Majorana L.)), Massaranduba Balata (Manilkara Huberi (Ducke) Chevalier, Massaranduba Balata, Solvent-Free Resin Extract, Massaranduba Chocolate (Manilkara Solimoesensis Gilly), Massoia Bark Oil, Mastic Gum, Mate, Absolute (Ilex Paraguariensis St. Hil.), Mate, Leaves, Mendaiol Sodium Diphosphate, Menhaden Oil, Menhaden Oil, Hydrogenated, Menhaden Oil, Partially Hydrogenated, D-2,8-P-Menthadien-1-O1, Menthadienol, P-Mentha-1,8-Dien-7-O1, Cis- And Trans-P-1(7),8-Menthadien-2-Y1Acetate, P-Menthan-2-One, P-Mentha-8-Thiol-3-One, P-Menth-1-Ene-9-A1, 8-P-Menthene-1,2-Diol, 1-P-Menthene-8-Thiol, P-Menth-1-En-3-O1, P-Menth-3-En-1-O1, 1-P-Meenthen-9-Y1 Acetate, Menthol, Menthone, Menthone 1,2-Glycerol Ketal, L-Menthone 1,2-Glycerol Ketal, Menthone-8-Thioacetate, 2-(L-Menthoxy)Ethanol, 3-(L-Menthoxy)-2-Methylpropane-1,2-Diol, 3-L-Menthoxypropane-1,2-Diol, Mentyl Acetate, L-Menthyl Acetoacetate, L-Menthyl Ethylene Glycol Carbonate, Menthyl Formate, L-Menthyl (R,S)-3-Hydroxybutyrate, Mentyl Isovalerate, L-Menthyl Lacate, 1-Menthyl Methyl Ether, 2-[2-(P-Menthyloxy)Ethoxy]Ethanol, Menthyl Propionate, Mentyl Propylene Glycol Carbonate, L-Menthyl 1,2-Propylene Glycol Carbonate, Menthyl Pyrrolidonecarboxylate, Mentyl Valerate, 2-Mercaptoanisole, 2-Mercapto-3-Butanol, 3-Mercapto-2-Butanone, (+/−)-3-Mercapto-1-Butyl Acetate, 2-Mercaptoethanol, 3-Mercaptoheptyl Acetate, 3-Mercaptohexanal, 3-Mercaptohexanol, 3-Mercaptohexyl Acetate, 3-Mercaptohexyl Butyrate, 3-Mercaptohexyl Hexanoate, 4-Mercapto-3-Methyl-2-Butanol, 3-Mercapto-2-Methyl-1-Butanol, 3-Mercapto-3-Methyl-1Butanol, 3-Mercapto-3-Methyl-1Butyl Acetate, 3-Mercapto-3-Methylbutyl Formate, 3-Mercapto-3-Methylbutyl Isovalerate, 4-Mercapto-4-Methyl-2-Hexanone, 3-Mercapto-2-Methylpentanal, 3-Mercapto-2-Methyl-1-Pentanol, (+/−)-4-Mercapto-4-Methyl-2-Pentanol, 2-Mercapto-2-Methyl-1-Pentanol, 4-Mercapto-4-Methyl-2-Pentanone, 2-Mercaptomethylpyrazine, 3-Mercapto-2-Pentanone, 4-Mercapto-2-Pentanone, 2,3 Or 10-Mercaptopinane, 1-Mercapto-2-Propanone, 2-Mercaptopropionic Acid, 3-Mercaptopropionic Acid, Mesquite Wood Extract, Methacrylic Acid-Divinylbenzene Copolymer, Methional Diethyl Acetal, L-Methionine, D1-Methionine, Methionyl Butyrate, L-Methionylglycine, 2-Methoxyacetophenone, P-Methoxy-Alpha-Methylcinnamaldehyde, O-Methoxybenzaldehyde, P-Methoxybenzaldehyde, 4-Methoxybenzoic Acid, 3-Methoxybenzoic Acid, 2-Methoxybenzoic Acid, P-Methoxycinnamaldehyde, O-Methoxycinnamaldehyde, Trans- And Cis-1-Methoxy-1-Decene, (S1)-Methoxy-3-Heptanethiol, 2-Methoxy-(3 Or 5 Or 6)-Isopropylpyrazine, N1-(2-Methoxy-4-Methylbenzyl)-N2-(2-(5-Methylpyridin-2-Y1)Ethyl)Oxalamide, N1-(2-Methoxy-4-Methylbenzyl)-N2-(2-(Pyridin-2-Y-1) Ethyl)Oxalamide, 4-Methoxy-2-Methyl-2-Butanethiol, 2-Methoxy-4-Methylphenol, (2 Or 5 Or 6)-Methoxy-3-Methylpyrazine (Mixture Of Isomers), 4-(P-Methoxyphenyl)-2-Butanon e, 1-(P-Methoxyphenyl)-1-Penten-3-One, 2-Methoxy-6-(2-Propenyl)Phenol, 2-Methoxy-4-Propylphenol, 2-Methoxy-3-(1-Methylpropyl)Pyrazine, Methoxypyrazine, 2-Methoxypyridine, 6-Methoxyquinoline, 2-Methoxy-4-Vinylphenol, Methyl Methacrylate, Methyl Acetate, 4′-Methylacetophenone, 2-Methylacetophenone, Methyl 1-Acetoxycyclohexyl Ketone, (+/−)-Methyl 5-Acetoxyhexanoate, Methyl 3-Acetoxy-2-Methylbutyrate, Methyl 3-Acetoxyoctanoate, Methyl N-Acetylanthranitate, 1-Methyl-2-Acetylpyrrole, 2-Methyl-3-(2-Furyl)Acrolein, Methyl Acrylate, Methyl Acrylate-Divinylbenzene, Completely Hydrolyzed, Copolymer, Methyl Acrylate-Dvb-Acrylonitrile, Completely Hydrolyzed, Terpolymer, Methyl Acrylate-Dvb (3.5%), Copolymer, Aminolyzed With Dmapa, Methyl Acrylate-Dvb (2%), Copolymer, Aminolyzed With Dmapa, Methyl Acrylate-Dvb-(Deg-Divinyl Ether)), Aminolyzed And Quarternized, Terpolymer, Methyl Acrylate-Dvb-(Deg-Divinyl Ether), Aminolyzed, Terpolymer, Methyl Alcohol, 2-Methylallyl Butyrate, Methyl-Alpha-Ionone, Methyl Anisate, P-Methylanisole, O-Methylanisole, Methyl Anthranilate, Methylated Silica, 4-Methylbenzaldehyde Propyleneglycol Acetal, Methyl Benzoate, 2-Methylbenzofuran, S-Methyl Benzothioate, 2-Methyl-4,5-Benzoxazole, Alpha-Methylbenzyl Acetate, Methylbenzyl Acetate (Mixed O-, M-, P-), Alpha-Methylbenzyl Alcohol, 4-Methylbenzyl Alcohol, Methyl Benzyl Disulfide, Alpha-Methylbenzyl Formate, Alpha-Methylbenzyl Propionate, 4-Methylbiphenyl, 3-Methylbutanethiol, 3-Methyl-2-Butanethiol, 2-Methyl-1-Butanethiol, 3-Methyl-2-Butanol, 2-Methyl-1-Butanol, 2-Methyl-2-Butenal, 3-Methyl-2-Butenal, (Z)-3-Hexenyl(E)-2-Methyl-2-Butenoate, Trans-2-Methyl-2-Butenoic Acid, 2-Methylbut-2-En-1-O1, 3-Methyl-2-Buten-1-O1, 2-Methyl-3-Buten-2-O1, 3-Methyl-3-Buten-2-One, 3-Methyl-3-Butenyl Acetate, 2-Methylbutyl Acetate, 2-Methylbutylamine, 2-Methylbutyl Isovalerate, 2-Methylbutyl 3-Methyl-2-Butenoate, 2-Methylbutyl 2-Methylbutyrate, 3-Methylbutyraldehyde, 2-Methylbutyraldehyde, Methyl Butyrate, 2-Methylbutyric Acid, Alpha-Methylcinnamaldehyde, P-Methylcinnamaldehyde, Methyl Cinnamate, 4-Methyl-Cis-2-Pentene, 6-Methylcoumarin, 3-Methylcrotonic Acid, 2-Methyl-1,3-Cyclohexadiene, 1-Methyl-2,3-Cyclohexadione, Methyl Cyclohexanecarboxylate, 4-Methylcyclohexanone, 3-Methylcyclohexanone, 2-Methylcyclohexanone, 3-Methyl-2-Cyclohexen-1-One, N-(2-Methylcyclohexyl)-2,3,4,5,6-Pentafluorobenzamide, 3-Methyl-1-Cyclopentadecanone, Methyleyclopentenolone, 1-Methyl-1-Cyclopenten-3-One, Methyl(E)-2-(Z)-4-Decadienoate, 3-Methyl-Gamma-Decalactone, Gamma-Methyldecalactone, Methyl 2-Decenoate, Methyl-Delta-Ionone, 5h-5-Methyl-6,7-Dihydrocyclopenta(B)Pyrazine, 2-Methyl4,5-Dihydrofuran-3-Thiol, Methyl Dihydrojastnonate, 4-Methyl-2,6-Dimethoxyphenol, Methyl N,N-Dimethylanthranilate, Methyl 3,7-Dimethyl-6-Octenoate, Methyl Disulfide, 2-Methyl-1,3-Dithiolane, 1-(Methyldithio)-2-Propanone, Methylene Chloride, 4-(3,4-Methylenedioxyphenyl)-2-Butanone, 3-(3,4-Methylenedioxyphenyl)-2-Methylpropanal, Methyl Esters Of Fatty Acids (Edible), 2-Methyl-(3 Or 5 Or 6)-Ethoxypyrazine, Methyl Ethyl Sulfide, Methyl Ethyl Trisulfide, Methyl N-Formlanthranilate, 2-Methylfuran, 2-Methyl-3-(Methylthio)Furan, 2-Methyl -5-(Methylthio)Furan, 5-Methyl-3(2h)-Furanone, 2-Methyl-3-Furanthiol, Methyl Furfuracrylate, 5-Methylfurfural, 5-Methylfurfuryl Alcohol, Methyl Furfuryl Disulfide, 5-Methylfurfurylmercaptan, Methyl 3-(Furfurthio)Propionate, Methyl 2-Furoate, 3-(5-Methyl-2-Furyl)-Butanal, 2-Methyl-3-Furyl 2-Methyl-3-Tetrahydrofuryl Disulfide, 2-Methyl-3-Furyl Methylthiomethyl Disulfide, 3-(5-Methyl-2-Furyl)Prop-2-Enal, 3-[(2-Methyl-3-Furyl)Thio]Butanal, (+/−)-3-[(2-Methyl-3-Furyl)Thio]-2-Butanone, 3-((2-Methyl-3-Furyl)Thio-4-Heptanone, Methyl Glucoside-Coconut Oil Ester, 6-Methyl-3,5-Heptadien-2-One, 6-Methylheptanal, Methyl Heptanoate, 2-Methytheptanoic Acid, 2-Methyl-3-Heptanone, 6-Methyl-5-Hepten-2-O1, 5-Methyl-2-Hepten-4-One, 6-Methyl.-3-Hepten-2-One, Trans-, 6-Methyl-5-Hepten-2-One, 6-Methyl-5-Hepten-2-One Propyleneglycol Acetal, 6-Methyl-5-Hepten-2-Y1 Acetate, 3-Methylhexanal, 5-Methyl-2,3-Hexanedione, S-Methyl Hexanethioate, Methyl Hexanoate, 2-Methylhexanoic Acid, 5-Methylhexanoic Acid, Methyl 3-Hexenoate, Methyl Cis-3-Hexenoate, Methyl 2-Hexenoate, 5-Methyl-5-Hexen-2-One, 5-Methyl-3-Hexen-2-One, 5-Methylhexyl Acetate, Methyl Hexyl Ether, 1-Methyl-1h-Pyrrole-2-Carboxaldehyde, Methyl P-Hydroxybenzoate, Methyl 3-Hydroxybutyrate, Methyl 3-Hydroxyhexanoate, Methyl 2-Hydroxy-4-Methylpentanoate, Alpha-Methyl-Beta-Hydroxypropyl Alpha-Methyl-Beta-Mercaptopropyl Sulfide, Methyl-Beta-Ionone, Methyl Isobutanethioate, Methyl Isobutyl Ketone, Methyl Isobutyrate, Methyl Isopentyl Disulfide, 2-Methyl-5-Isopropylpyrazine, Methyl Isothiocyanate, Methyl Isovalerate, Methyl Jasmonate, Methyl Laurate, Methyl Levulinate, Methyl Linoleate (48%) Methyl Linolenate (52%) Mixture, Methyl Mercaptan, Methyl 3-Mercaptobutanoate, Methyl O-Methoxybenzoate, 1-Methyl-3-Methoxy-4-Isopropylbenzene, 2-Methyl-5-Methoxythiazole, Methyl N-Methylanthranilate, S-Methyl 3-Methylbutanethioate, Methyl 3-Methyl-1-Butenyl Disulfide, 3-Methyl-2(3-Methylbut-2-En-1-Y1)Furan, Methyl 2-Methylbutyrate, Methyl 2-Methyl-3-Furyl Disulfide, S-Methyl 4-Methylpentanethioate, Methyl 2-Methylpentanoate, Methyl 2-Methylphenyl Disulfide, Methyl (Methylthio)Acetate, Methyl 3-(Methylthio)Butanoate, 2-Methyl-1-Methylthio-2-Butene, Methyl 4-(Methylthio)Butyrate, Methyl 2-Methylthiobutyrate, Methyl (Methylthio)Methyl Disulfide, 4-Methyl-2-(Methylthiomethyl)-2-Hexenal, 5-Methyl-2-(Methylthiomethyl)-2-Hexenal, 4-Methyl-2-(Methylthiomethyl)-2-Pentenal, Methyl 3-Methylthiopropionate, Methyl 4-Methylvalerate, Methyl Myristate, 1-Methylnaphthalene, Methyl Beta-Naphthyl Ketone, Methyl Nicotinate, 3-Methyl-2,4-Nonanedione, Methyl Nonanoate, 4-Methylnonanoic Acid, Methyl 2-Nonenoate, Methyl 3-Nonenoate, Methyl 2-Nonynoate, 2-Methyloctanal, (+/−)-6-Methyloctanal, Methyl Octanoate, 4-Methyloctanoic Acid, 2-Methyl-2-Octenal, Methyl 2-Octenoate, Methyl Cis-4-Octenoate, Methyl Trans-2-Octenoate, Methyl Cis-5-Octenoate, (E)-7-Methyl-3-Octen-2-One, Methyl Octyl Sulfide, Methyl 2-Octynoate, 2-Methyl-3 Or 5 Or 6-(Furfurylthio)Pyrazine (Mixture Of Isomers), 3-Methyl-2-Oxobutanoic Acid, Methyl 2-Oxo-3-Methylpentanoate, 3-Methyl-2-Oxopentanoic Acid, 4-Methyl-2-Oxopentanoic Acid, 2-Methylpentanal, 4-Methyl-2,3-Pentanedione, 4-Methylpentanoic Acid, 3-Methylpentanoic Acid, 3-Methyl-1-Pentanol, 4-(Methylthio)-4-Methyl-2-Pentanone, 2-Methyl-2-Pentenal, 4-Methyl-2-Pentenal, Methyl 4-Pentenoate, 2-Methyl-4-Pentenoic Acid, 4-Methylpent-2-Enoic Acid, 2-Methyl-3-Pentenoic Acid, 2-Methyl-2-Pentenoic Acid, 1-(4-Methoxyphenyl)-4-Methyl-1-Penten-3-One, 4-Methyl-3-Penten-2-One, 4-Methyl-4-Penten-2-One, 4-Methyl-2-Pentyl-1,3-Dioxolane, 4-Methylpentyl Isovalerate, Beta-Methylphenethyl Alcohol, Alpha-Methylphenethyl Butyrate, Methyl Phenethyi Ether, Methyl Phenylacetate, 2-Methyl-4-Phenyl-2-Butanol, 3-Methyl-4-Phenyl-3-Butene-2-One, 2-Methyl-4-Phenyl-2-Butyl Acetate, 2-Methyl-4-Phenyl-2-Butyl Isobutyrate, 2-Methyl-4-Phenylbutyraldehyde, 3-Methyl-2-Phenylbutyraldehyde, Methyl 4-Phenylbutyrate, Methyl Phenyl Disulfide, Methyl Beta-Phenylglycidate, 3-Methyl-3-Phenyl Glycidic Acid Ethyl Ester, 5-Methyl-2-Phenyl-2-Hexenal, 4-Methyl-1-Phenyl-2-Pentanone, 4-Methyl-2-Phenyl-2-Pentenal, Methyl 3-Phenylpropionate, Methyl Phenyl Sulfide, 2-Methylpiperidine, Methylpolysilicone, 2-Methyl-2-(Methyldithio)Propanal, S-Methyl Propanethioate, 2-Methyl-1-Propanethiol, Methyl Propenyl Disulfide, Methyl 1-Propenyl Sulfide, 2-Methyl-3-(P-Isopropylphenyl) Propionaldehyde, Methyl Propionate, 3-Methyl-5-Propyl-2-Cyclohexen-1-One, Methyl Propyl Disulfide, 2-Methylpropyl-3-Methylbutyrate, (?)-4-Methyl-2-Propyl-1,3-Oxathiane, 2-Methyl-4-Propyl-1,3-Oxathiane, 2-(2-Methylpropyl)Pyridine, 2-(1-Methylpropyl)Thiazole, Methyl Propyl Trisulfide, Methyl-(3 Or 5 Or 6)-(Methylthio)Pyrazine (Mixture Of Isomers), 2-Methylpyrazine, Methyl 2-Pyrrolyl Ketone, 6-Methylquinoline, 5-Methylquinoxaline, Methyl Salicylate, Methyl Sorbate, Methyl Sulfide, Methyl P-Tert-Butylphenylacetate, 2-Methyltetrahydrofuran-3-One, 2-Methyl-3-Tetrahydrofuranthiol, (?)-2-Methyltetrahydrofuran-3-Thiol Acetate, 7-Methyl-4,4a,5,6-Tetrahydro-2(3h)-Naphthalenone, 2-Methyltetrahydrothiophen-3-One, 4-Methylthiazole, 4-Methyl-5-Thiazoleethanol, 4-Methyl-5-Thiazoleethanol Acetate, 4-Methyl-3-Thiazoline, 2-(4-Methyl-5-Thiazolyl)Ethyl Butanoate, 2-(4-Methyl-5-Thiazolyl)Ethyl Decanoate, 2-(4-Methyl-5-Thiazolyl)Ethyl Formate, 2-(4-Methyl-5-Thiazolyl)Ethyl Hexanoate, 2-(4-Methyl-5-Thiazolyl)Ethyl Isobutyrate, 2-(4-Methyl-5-Thiazolyl)Ethyl Octanoate, 2-(4-Methyl-5-Thiazolyl)Ethyl Propionate, 2-Methylthioacetaldehyde, S-Methyl Thioacetate, 2-Methyl-3-Thioacetoxy-4,5-Dihydrofuran, Methylthio 2-(Acetyloxy)Propionate, 3-(Methylthio)Butanal, 4-(Methylthio)Butanol, 1-(Methylthio)-2-Butanone, 4-(Methylthio)-2-Butanone, 3-(Methylthio)-2-Butanone, 4-(Methylthio)Butyl Isothiocyanate, Methyl Thiobutyrate, 1-(3-(Methylthio)-Butyryl)-2,6,6-Trimethylcyclohexene, 2-(Methylthio)Ethanol, 2-(Methylthio)Ethyl Acetate, Methyl 2-Thiofuroate, (+/−)-3-(Methylthio)Heptanal, 3-(Methylthio)-1-Hexanol, 3-Methylthiohexenal, 3-(Methylthio)Hexyl Acetate, 6-(Methylthio)Hexyl Isothiocyanate, 2-((Methylthio)Methyl)-2-Butenal, Methylthiomethyl Butyrate, Methylthiomethyl Hexanoate, Methylthiomethylmercaptan, 2-(Methylthiomethyl)-3-Phenylpropenal, 3-(Methylthio)Methylthiophene, 1-(Methylthio)-3-Octanone, 4-(Methylthio)-2-Oxobutanoic Acid, 4-(Methylthio)-2-Pentanone, 5-(Methylthio)Pentyl Isothiocyanate, 5-Methyl-2-Thiophenecarboxaldehyde,O-(Methylthio)Phenol, 1-Methylthio-2-Propanone, Methylthio 2-(Propionyloxy)Propionate, 3-(Methylthio)Propyl Acetate, 3-(Methylthio)Propylamine, 3-(Methylthio)Propyl Hexanoate, 3-Methylthiopropyl Isothiocyanate, 3-(Methylthio)Propyl Mercaptoacetate, 2-Methyl-3-Tolylpropionaldehyde (Mixed O-, M-, P-), 12-Methyltridecanal, 3-Methyl-1,2,4-Trithiane, 2-Methylundecanal, Methyl 9-Undecenoate, Methyl 10-Undecenoate, Methyl 2-Undecynoate, Methyl Valerate, 2-Methylvaleric Acid, 2-Methyl-5-Vinylpyrazine, (+/−)-2-(5-Methyl-5-Vinyttetrahydrofuran-2-Y1 )Propionaldehyde, 4-Methyl-5-Vinylthiazole, Michelia Alba, Extract, Microparticulated Protein Product, Milk, Skim Milk, Nonfat Milk, Reduced-Fat Milk, Milk Clotting Enzyme, Aspergillus Oryzae Recombinant, Milk Clotting Enzyme From Bacillus Cereus (Frankland And Frankland), Milk Clotting Enzyme From Endothia Parasitica, Clotting Enzyme From Mucor Michel Cooney Et Emerson, Milk Clotting Enzyme From Mucor Pusillus L., Milk Powder, Whole, Enzyme-Modified, Mimosa, Absolute (Acacia Decurrens Var. Dealbata), Mimosa Concrete (Acacia Decurrens Willd, Var, Dearbata), Mineral Oil, White, Mintlactone, Mixture Of 3,6-Diethyl-1,2,4,5-Tetrathiane And 3,5-Diethyl-1,2,4-Trithiolane, Mixture Of Methyl Cyclohexadiene And Methylene Cyclohexene, Mixture Of Butyl. Propyl Disulfide And Propyl And Butyl Disulfide, Molasses, Concentrate, Molasses, Extract (Saccharum Officinarum L.), Molasses (Saccharum Officinarum L.), Molecular Sieve Resins, Monoammonium Glutamate, Mono- And Diglycerides, Mono- And Diglycerides, Acetic Acid Esters And Sodium And Calcium Salts, Mono- And Diglycerides, Acetyltartaric Acid Esters And Sodium And Calcium Salts, Mono- And Diglycerides, Citric Acid Esters And Sodium And Calcium Salts, Mono- And Diglycerides, Diacetyltartaric Acid Esters, Mono- And Diglycerides, Ethoxylated, Mono- And Diglycerides, Lactic Acid Esters And Sodium And Calcium Salts, Mono- And Diglycerides, Monosodium Phosphate Derivatives, Mono- And Diglycerides, Sodium Sulfoacetate Derivatives, Mono-, Di-, And Triglycerides, Monoethanolamine, Monoglyceride Citrate, Monoglycerides, Acetylated, Monoisopropyl Citrate, Monomenthyl Glutarate, L-, Monomenthyl Succinate, Monopotassium Glutamate, Monosodium Glutamate, Montan Wax Fatty Acids, Oxidatively Refined, Polyhydric Alcohol Diesters, Morpholine, Morpholine, Fatty Acid Salts, Mountain Maple (Acer Spicatum Lam.), Mountain Maple Bark (Acer Spicatum Lam), Mountain Maple, Extract Solid (Acer Spicatum Lam.), Mullein Flowers (Verbascum Spp.), Mushroom Oil, Distilled, Musk Ambrette, Musk, Ketone, Musk Tonquin (Moschus Moschiferus L.), Mustard, Brown (Brassica Spp.), Mustard, Brown, Extract (Brassica Spp.), Mustard Flour, Mustard Oil, Mustard, Oriental, Mustard, Yellow (Brassica Spp.), Mustard, Yellow, Extract (Brassica Spp.), Beta-Myrcene, Myrcenyl Methyl Ether, Myricitrin, Myristaldehyde, Myristic Acid, Myristyl Alcohol, Myrrh, Extract, Myrrh, Gum (Commiphora Spp.), Myrrh, Oil (Commiphora Spp.), Myrtenol, Myrtenyl Acetate, Myrtle Leaves (Myrtus Communis L.), Myrtle, Oil (Myrtus Communis L.), Naphtha, 2-Naphthalenthiol, Beta-Naphthyl Anthranilate, Beta-Naphthyl Ethyl Ether, Beta-Naphthyl Isobutyl Ether, Beta-Naphthyl Methyl Ether, Naringin Dihydrochalcone, Naringin, Extract (Citrus Paradisi Macf.), Natamycin, Natural Gas, N-(1,1-Dimethyl-2-Hydroxyethyl)-2,2-Diethylbutanamide, Neohesperidin Dihydrochalcone, D-Neomenthol, Neotame, Nerol, Neroli, Bigarade Oil (Citrus Aurantium L.), Nerolidol, Nerolidol Oxide, Neryl Acetate, Neryl Butyrate, Neryl Formate, Neryl Isobutyrate, Neryl Isovalerate, Neryl Propionate, N-Ethyl-2,2-Diisopropylbutanamide, N-(2-Hydroxyethyl)-2,3-Dimethyl-2-Isopropylbutanamide, Niacin, Niacinamide, Nickel, Nicotinamide-Ascorbic Acid Complex, Niger Gutta (Ficus Platyphylla Del.), Nisin Preparation, Nispero, Nitrates, Sodium & Potassium, Nitrites, Sodium & Potassium, Nitrogen, Nitrogen Oxides, Nitrosyl Chloride, Nitrous Oxide, (+/−)-N-Lactoyityramine, 2,4-Nonadienal, 2-Trans-6-Trans-Nonadienal, 2,6-Nonadienal Diethyl Acetal, 2,4-Nonadien-1-O1, (E)-3-(Z)-6-Nonadien-1-O1, (Z)(Z)-3,6-Nonadien-1-O1, 2,6-Nonadien-1-O1, (E,Z)-2,6-Nonadien-1-O1 Acetate, (E,Z)-3,6-Nonadien-1-O1 Acetate, Cis,Cis-3,6-Nonadienyl Acetate, Gamma-Nonalactone, Nonanal, Normal Dimethyl Acetal, Nonanal Propyleneglycol Acetal, 1,3-Nonanediol Acetate (Mixed Esters), 1,4-Nonanediol Diacetate, 1,9-Nonanedithiol, Nonanoic Acid, 2-Nonanol, 4((2-Methyl-3-Furyl)Thio)-5-Nonanone, 2-Nonanone, 3-Nonanone, 2-Nonanone Propyleneglycol Acetal, 3-Nonanon-1-O1, 3-Nonanon-1-Y1 Acetate, Nonanoyl 4-Hydroxy-3-Methoxybenzylamide, Nona-2,4,6-Trienal, Cis-6-Nonenal, 2-Nonenal, 1-Nonene, (E)-2-Nonenoic Acid, 2-Nonenoic Acid Gamma-Lactone, Cis-6-Nonen-1-O1, Trans-2-Nonen-1-O1, Cis-2-Nonen-1-O1, 3-Nonen-2-One, 8-Nonen-2-One, 5-Nonen-Trans-2-One, Cis-6-Nonenyl Acetate, Cis-3-Nonenyl Acetate, 3-Nonyl Acetate, Nonyl Acetate, Nonyl Alcohol, Nonyl Isovalerate, Nonyl Octanoate, Nootkatone, N-P-Benzeneacetonitritementhanecarboxamide, N-(2-(Pyridin-2-Y1)Ethyl)-3-P-Menthanecarboxamide, Nutmeg (Myristica Fragrans Houtt.), Nutmeg, Oil (Myristica Fragrans Houtt.), Nutmeg Oleoresin, Oak Chips, White, Extract (Quercus Alba L.), Oak Moss, Absolute (Evernia Spp.), Oak Moss, Concrete (Evernia Prunasti Spp.), Oak Wood, English (Quercus Robur L.), Oat Gum, Ocimene, 9,12-Octadecadienoic Acid (48%) And 9,12,15-Octadecatrienoic Acid (52%), Delta-Octadecalactone, Gamma-Octadecalactone, 9-Octadecenal, Octadecylamine, 2-Trans-6-Trans-Octadienal, Trans,Trans-2,4-Octadiena , (E,E)-2,4-Octadien-1-01, 3,5-Octadien-2-One, Trans, Trans-, 1,5-Octadien-3-One, Octafluorocyclobutane, Octahydro-4,8a-Dimethyl-4a(2h)-Naphthol, Octahydrocoumarin, Delta-Octalactone, Gamma-Octalactone, Octanal, Octanal Dimethyl Acetal, Octanal Propyleneglycol Acetal, 4,5-Octanedione, 2,3-Octanedione, 1,8-Octanedithiol, Octanoic Acid, 3-Octanal, 2-Octanol, 1-Octanol, 2-Octanone, 3-Octanone, 3-(Hydroxymethyl)-2-Octanone, 6-Octenal, 2-Octenal, Cis-5-Octenal, 1-Octene, 3-Octenoic Acid, (E)-2-Octenoic Acid, (E)-2-Octen-1-O1, Cis-3-Octen-1-O1, 1-Octen-3-O1, (E)-2-Octen-4-O1, Cis-5-Octen-1-O1, Trans-3-Octen-2-O1, 3-Octen-2-One, 2-Octen-4-One, 1-Octen-3-One, 4-Octen-3-One, Z-5-Octenyl Acetate, 1-Octen-3-Y1 Acetate, Trans-2-Octen-1-Y1 Acetate, Trans-2-Octen-1-Y1 Butanoate, 1-Octen-3-Y1 Butyrate, (E)-2-(2-Octenyl)Cyclopentanone, Cis-3-Octenyl Propionate, (Z)-5-Octenyl Propionate, 1-Octenyl Succinic Anhydride, 3-Octyl Acetate, Octyl Acetate, Octyl Alcohol, Synthetic, Octyl Butyrate, 3-Octyl Butyrate, Octyl Formate, 3-Octyl Formate, Octyl 2-Furoate, Octyl Gallate, Octyl Heptanoate, Octyl Isobutyrate, Octyl Isovalerate, Octyl 2-Methylbutyrate, Octyl Octanoate, Octyl Phenylacetate, Octyl Propionate, (?)-6-Octyltetrahydro-2h-Pyran-2-One, O-Ethyl S-(2-Furylmethyl)Thiocarbonate, Oiticica Oil, Oleic Acid, Oleic Acid, From Tall Oil Fatty Acids, Olestra, Oleyl Alcohol, Olibanum, Absolute (Boswellia Spp.), Olibanum, Gum, Resin (Boswellia Spp.), Olibanum, Oil (Boswellia Spp.), Olibanum, Resinoid (Boswellia Spp.), Onion, Oil (Allium Cepa L.), Opopanax, Gum, Opopanax, Non-Specific, Opopanax, Oil, Opopanax Tincture, Orange B, Orange Essence, Natural, Orange Essence Oil, Natural, Orange, Extract, Orange Flowers, Absolute (Citrus Aurantium L.), Orange Flowers, Bitter (Citrus Aurantium L), Orange, Juice, Orange Leaf, Absolute (Citrus Aurantium L.), Orange, Oil, Distilled (Citrus Sinensis (L.) Osbeck), Orange, Oil, Terpeneless (Citrus Sinensis (L.) Osbeck), Orange Peel, Orange Peel, Bitter, Extract (Citrus Aurantium L.), Orange Peel, Bitter, Oil (Citrus Aurantium L.), Orange Peel, Sweet, Extract (Citrus Sinensis (L.) Osbeck), Orange Peel, Sweet, Oil (Citrus Sinensis (L.) Osbeck), Orange Peel, Sweet, Oil, Terpeneless (Citrus Sinensis (L.) Osbeck), Oregano, European (Origanum Spp.), Oregano (Lippia Spp., Usually L. Graveolens Hbk), Oregano (Other Genera Including Coleus, Lantana And Hyptis), Origanum Oil (Extractive) (Thymus Capitatus Hoff. Et Link), Orin Lactone, L-Ornithine Monochlorohydrate/Ornithine, Orris, Concrete, Liquid, Oil (Iris Florentina L.), Orris Root, Extract (Iris Florentina L.), Osmanthus Absolute, Ox Bile Extract, Oxirane (Chloromethyl)-, Polymer With Ammonia, Reaction Product With Chloromethane, 3-Oxobutanal, Dimethyl Acetal, 5-Oxodecanoic Acid, 3-Oxodecanoic Acid Glyceride, 5-Oxododecanoic Acid, 3-Oxododecanoic Acid Glyceride, 2-Oxo-3-Ethyl-4-Butanolide, 3-Oxohexadecanoic Acid Glyceride, 3-Oxohexanoic Acid Diglyceride, 5-Oxooctanoic Acid, 3-Oxooctanoic Acid Glyceride, 2-Oxopentanedioic Acid, 2-Oxo-3-Phenylpropionic Acid, 3-Oxotetradecanoic Acid Glyceride, 2-Oxothiolane, Oxystearin, Ozone, Palmitic Acid, Pancreatin, Pansy (Viola Tricolor L.), D-Pantothenamide, D-Pantothenyl Alcohol, Papain (Carica Papaya L.), Paprika (Capsicum Annuum L.), Paprika Oleoresin (Capsicum Annuum L.), Paraffin And Succinic Derivatives, Synthetic, Paraffin Wax, Paraldehyde, Cheese, Cheddar Cheese, Cheese Powder, Parmesan Cheese, Reggiano Cheese, Parsley, Oil (Petroselinum Spp.), Parsley, Oleoresin (Petroselinum Spp.), Parsley (Petroselinum Spp.), Passion Flower Extract, Passion Flower (Passiflora Incarnata L.), Patchouly, Oil (Pogostemon Spp.), Peach Kernel, Extract (Prunus Persica Sieb Et Zucc.), Peach Leaves, Extract (Prunus Persica (L.) Batsch), Peach Leaves (Prunus Persica (L.) Basch), Peanut Oil., Peanut Stearine (Arachis Hypogaea L.), Pecan Shell Flour, Pectin, Pectin, Amidated, Pectinase From Aspergillus Niger, Pectinase From Bacillus Subtilis, Pectin, Modified, Peg Fatty Acid Esters And Mono-, Di-, And Triglycerides Mixture, Pendare (Couma Macrocarpa Barb. Rodr. &. Couma Utilis (Mart.) Muell Arg.), Penicillinase From Bacillus Subtilis, Penicillium Roqueforti, Pennyroyal, Oil, American (Hedeoma Pulegiodes (L.)), Pennyroyal, Oil, European (Mentha Pulegium L.), Omega-Pentadecalactone, Pentadecanoic Acid, 2-Pentadecanone, 2,4-Pentadienal, 2,3-Pentanedione, 3-Pentanethiol, 2-Pentanethiol, 1-Pentanethiol, 2-Pentanol, 2-Pentanone, 2-Pentanoylfuran, 2-Pentenal, 4-Pentenal, 2-Pentenoic Acid, 4-Pentenoic Acid, 1-Penten-3-O1, 2-Penten-1-O1, 3-Penten-2-One, 1-Penten-3-One, 4-Pentenyl Acetate, Pent-2-Enyl Hexanoate, 4-Pentenyl Isothiocyanate, 2-Pentenyl-4-Propyl-1,3-Oxathiane (Mixture Of Isomers), 2-Pentyl Acetate, Pentylamine, 2-Pentyl-1-Buten-3-One, 2-Pentyl Butyrate, 2-Pentylfuran, Pentyl 2-Furyl Ketone, 5-Pentyl-3h-Furan-2-One, 2-Pentyl-3-Methyl-2-Cyclopenten-1-One, 2-Pentyl 2-Methylpentanoate, 2-Pentylpyridine, 2-Pentylthiazole, 2-Pentylthiophene, Pepper, Black, Oil (Piper Nigrum L.), Pepper, Black, Oleoresin (Piper Nigrum L.), Pepper, Black (Piper Nigrum L.), Pepper, Cayenne, Peppermint Leaves (Mentha Piperita L.), Peppermint, Oil (Mentha Piperita L.), Peppermint Plant, Pepper, Red, Pepper, White, Oil (Piper Nigrum L.), Pepper, White, Oleoresin (Piper Nigrum L.), Pepper, White (Piper Nigrum L.), Pepsin, Peptones, Peracetic Acid, Perillaldehyde, Perillaldehyde Propyleneglycol Acetal, Perilla Leaf Oil, Perillo, Perillyl Acetate, Periodic Acid, Petitgrain, Lemon, Oil (Citrus Limon (L.) Burm. F.), Petitgrain, Mandarin, Oil (Citrus Reticulata Blanco Var. Mandarin), Petitgrain, Oil (Citrus Aurantium L.), Petrolatum, Petroleum Hydrocarbons, Odorless, Light, Petroleum Naphtha, Petroleum Wax, Petroleum Wax, Synthetic, Phaffia Yeast, Alpha-Phellandrene, Phenethyl Acetate, Phenethyl Alcohol, Phenethylamine, Phenethyl Anthranilate, Phenethyl Benzoate, Phenethyl Butyrate, Phenethyl Cinnamate, Phenethyl Decanoate, Phenethyl Formate, Phenethyl 2-Furoate, Phenethyl Hexanoate, Phenethyl Isobutyrate, Phenethyl Isothiocyanate, Phenethyl Isovalerate, Phenethyl 2-Methylbutyrate, Phenethyl Octanoate, Phenethyl Phenylacetate, Phenethyl Propionate, Phenethyl Salicylate, Phenethyl Senecioate, Phenethyl Tiglate, Phenol, Phenol-Formaldehyde, Cross-Linked, Tetraethylenepentamine Activated, Phenol-Formaldehyde, Cross-Linked, Triethylenetetramine Activated, Phenol-Formaldehyde, Cross-Linked, Triethylenetetramine & Tetraethylenepentamine Activated, Phenol-Formaldehyde, Sulfite-Modified, Cross-Linked, Phenoxyacetic Acid, 2-Phenoxyethyl Isobutyrate, 2-Phenoxyethyl Propionate, Phenylacetaldehyde, Phenylacetaldehyde 2,3-Butylene Glycol Acetal, Phenylacetaldehyde Diethyl Acetal, Phenylacetaldehyde Diisobutyl Acetal, Phenylacetaldehyde Dimethyl Acetal, Phenylacetaldehyde Glyceryl Acetal, Phenylacetaldehyde Propyleneglycol Acetal, Phenyl Acetate, Phenylacetic Acid, D1-Phenylalanine, L-Phenylalanine, 4-Phenyl-2-Butanol, 2-Phenyl-2-Butenal, 4-Phenyl-3-Buten-2-O1, 4-Phenyl-3-Buten-2-One, 4-Phenyl-2-Butyl Acetate, Phenyl Butyrate, 2-Phenyl-3-Carbethoxy Furan, Phenyl Disulfide, (+/−)-1-Phenylethylmercaptan, Phenylethyl Mercaptan, (+/−)-2-Phenyl-4-Methyl-2-Hexenal, 1-Phenyl-3-Methyl-3-Pentanol, 1-Phenyl-3 Or 5-Propylpyrazole, 5-Phenylpentanol, 2-Phenyl-4-Pentenal, 3-Phenyl-4-Pentenal, O-Phenylphenol, 1-Phenyl-1,2-Propanedione, 3-Phenyl-1-Propanol, 1-Phenyl-1-Propanol, 2-Phenyl-3-(2-Furyl)-Prop-2-Enal, 3-Phenylpropionaldehyde, 2-Phenylpropionaldehyde, 2-Phenylpropionaldehyde Dimethyl Acetal, 3-Phenylpropionic Acid, 3-Phenylpropyl Acetate, 2-Phenylpropyl Butyrate, 3-Phenylpropyl Cinnamate, 3-Phenylpropyl Formate, 3-Phenylpropyl Hexanoate, 2-Phenylpropyl Isobutyrate, 3-Phenylpropyl Isobutyrate, 3-Phenylpropyl Isovalerate, 3-Phenylpropyl Propionate, 2-(3-Phenylpropyl)Pryridine, 2-(3-Phenylpropyl)Tetrahydrofuran, Phenyl Salicylate, Phosphoric Acid, Phosphorus Oxychloride, Phthalide, Phytol, Phytyl Acetate, Pimenta Leaf, Oil (Pimenta Officinalis Lindl.), 3-Pinanone, Pine Bark, White, Extract Solid (Pinus Strobus L.), Pine Bark, White, Oil (Pinus Strobus L.), Pine Bark, White (Pinus Strobus L.) Alpha-Pinene, Beta-Pinene, Pine Needle, Dwarf, Oil (Pinus Mugo Turra Var. Pumilio (Haenke) Zenari), Pine, Scotch, Oil (Pinus Sylvestris L.), Pine Tar, Oil (Pinus Spp.), Pine, White, Oil (Pinus Spp.), Pinocarveol, Pinocarvyl Isobutyrate, Piperazine, Piperazine Dihydrochloride, Piperidine, Piperine, Piperitenone, Piperitenone Oxide, L-Piperitone, D-Piperitone, Piper Longum Distillate, Piperonal, Piperonal Propyleneglycol Acetal, Piperonyl Acetate, Piperonyl Isobutyrate, Pipsissewa Leaves, Extract (Chimaphila Umbellata Nutt.), 1,3-P-Menthadien-7-A1, P-Menthane-3,8-Diol, P-Menthan-7-O1, P-Menth-1-En-9-O1, Polyacrylamide, Polyacrylamide Resin, Modified, Poly(Acrylic Acid-Co-Hypophosphite), Sodium Salt, Polyacrylic Acid, Sodium Salt, Poly(Alkyl(C16-22)Acrylate), Polydextrose, Poly(Divinylbenzene-Co-Ethylstyrene), Poly(Divinylbenzene-Co-Trimethyl(Vinylbenzyl)Ammonium Chloride), Polyethylene (M W 2,000-21,000), Polyethylene Glycol (M W 200-9,500), Polyethylene Glycol (400) Dioleate, Polyethylene, Oxidized, Polyethylenimine Reaction Product W/1,2-Dichloroethane, Polyglycerol Esters Of Fatty Acids, Polyglyceryl Phthalate Ester Of Coconut Oil Fatty Acids, Polyisobutylene (Min M W 37,000), Polylimonene, Polymaleic Acid, Polymaleic Acid, Sodium Salt, Poly(Maleic Anhydride), Sodium Salt, Polyoxyethylene (600) Dioleate, Polyoxyethylene Dioleate, Polyoxyethylene (600) Monoricinoleate, Polyoxyethylene 40 Monostearate, Polypropylene Glycol (M W 1,200-3,000), Polysorbate 20, Polysorbate 60, Polysorbate 65, Polysorbate 80, Polystyrene, Cross-Linked, Chloromethylated, Then Aminated With Trimethylamine, Dimethylamine, Diethylenetriamine, Or Triethanolamine, Polyvinyl Acetate, Polyvinyl Alcohol, Polyvinyl Polypyrrolidone, Polyvinylpyrrolidone, Pomegranate Bark, Extract (Punica Granatum L.), Poplar Buds (Populus Spp.), Poppy Seed (Papaver Somniferum L.), Potassium Acetate, Potassium Acid Pyrophosphate, Potassium Acid Tartrate, Potassium Benzoate, Potassium Bicarbonate, Potassium Bisulfite, Potassium Borate, Potassium Bromate, Potassium Bromide, Potassium Caprate, Potassium Caprylate, Potassium Carbonate, Potassium Caseinate, Potassium Chloride, Potassium Citrate, 2-(1′-Ethoxy)Ethoxypropanoate, Potassium Fumarate, Potassium Gibberellate, Potassium Gluconate, Potassium Glycerophosphate, Potassium Hydroxide, Potassium Hypophosphate, Potassium Hypophosphite, Potassium Iodate, Potassium Iodide, Potassium Lactate, Potassium Laurate, Potassium Metabisulfite, Potassium N-Methyldithiocarbamate, Potassium Myristate, Potassium Nitrate, Potassium Nitrite, Potassium Oleate, Potassium Palmitate, Potassium Pectinate, Potassium Permanganate, Potassium Persulfate, Potassium Phosphate, Monobasic, Potassium Phosphate, Tribasic, Potassium Polymetaphosphate, Potassium Pyrophosphate, Potassium Salts Of Fatty Acids, Potassium Sorbate, Potassium Stearate, Potassium Sulfate, Potassium Sulfite, Potassium Tripolyphosphate, Potato Starch, Prenyl Acetate, Prenyl Benzoate, Prenyl Caproate, Prenyl Formate, Prenyl Isobutyrate, Prenyl Thioacetate, Prenylthiol, Prickly Ash Bark Extract (Xanthoxylum Spp.), Prickly Ash Bark, Oil, L-Proline, 1,2-(Di(1′-Ethoxy)Ethoxy)Propane, Propane, 1,3-Propanedithiol, 1,2-Propanedithiol, 1,1-Propanedithiol, 2-Propanethiol, 3-(Methylthio)Propanol, (2-Furyl)-2-Propatione, 1-(P-Methoxyphenyl)-2-Propanone, Di-(1-Propenyl)-Sulfide (Mixture Of Isomers), 4-Propenyl-2,6-Dimethoxyphenol, Propenylguaethol, (Z)-4-Propenylphenol, 4-(2-Propenyl)Phenyl-Beta-D-Glucopyranoside, Propenyl Propyl Disulfide, Propionaldehyde, 3-(Methylthio)Propionaldehyde, Propionic Acid, 2-(4-Methyl-2-Hydroxyphenyl)Propionic Acid-Gamma-Lactone, 2-Propionylpyrrole, 2-Propionylpyrroline, 2-Propionylthiazole, 2-Propionyl-2-Thiazoline, Propiophenone, Propyl Acetate, Propyl Alcohol, Propylamine, P-Propylanisole, Propyl Benzoate, Propyl Butyrate, Propyl Cinnamate, Propyl 2,4-Decadienoate, 4-Propyl-2,6-Dimethoxyphenol, 2-Propyl-4,5-Dimethyloxazole, Propyl Disulfide, Propylene Chlorohydrin, Propylene Glycol, Propylene Glycol Alginate, Propyleneglycol Diacetate, Propylene Glycol Dibenzoate, Propyleneglycol Dibutyrate, Propyleneglycol Dihexanoate, Propyleneglycol Di-2-Methylbutyrate, Propyleneglycol Dioctanoate, Propyleneglycol Dipropionate, Propylene Glycol Mono- And Diesters Of Fats And Fatty Acids, Propyleneglycol Monobutyrate, Propyleneglycol Monohexanoate, Propyleneglycol Mono-2-Methylbutyrate, Propylene Glycol Stearate, Propylene Oxide, Propyl Formate, Propyl 2-Furanacrylate, Propyl 2-Furoate, Propyl Gallate, Propyl Heptanoate, Propyl Hexanoate, Propyl P-Hydroxybenzoate, 3-Propylidenephthalide, Propyl Isobutyrate, Propyl Isovalerate, Propyl Levulinate, Propyl Mercaptan, Propyl 2-Mercaptopropionate, Propyl 2-Methyl-3-Furyl Disulfide, Alpha-Propylphenethyl Alcohol, P-Propylphenol, O-Propylphenol, Propyl Phenylacetate, Propyl Propane Thiosulfonate, Propyl Propionate, 2-Propylpyrazine, 2-Propylpyridine, Propyl Pyruvate, Propyl Sorbate, Propyl 4-Tert-Butylphenylacetate, Propyl Thioacetate, Protease From Aspergillus Flavus, Protease From Aspergillus Niger, Protease From Aspergillus Oryzae, Protease From Bacillus Amyloliquefaciens, Protease From Bacillus Licheniformis, Protease From Bacillus Subtilis, Protein, Animal, liydrolyzed, Protein Hydrolysate, Unspecified, Protein, Milk, Hydrolyzed, Protein, Vegetable, Hydrolyzed, Pseudoionone, Psyllium Seed Husk, Pulegone, Pulp, Pyrazine, Pyrazine Ethanethiol, Pyrazinyl Methyl Sulfide, 3-(2-Methylpropyl)Pyridine, Pyridine, 2-Pyridinemethanethiol, Pyridoxine, Pyridoxine Hydrochloride, Pyroligneous Acid, Pyroligneous Acid, Extract, Pyrrole, Pyrrolidine, Pyrrolidino-[1,2e]-4h-2,4-Dimethyl-1,3,5-Dithiazine, 1-Pyrroline, Pyruvaldehyde, Pyruvic Acid, Quassia, Extract (Picrasma Excelsa (Sw.) Planch Or Quassia Amara L.), Quaternary Ammonium Chloride Combination, Quebracho Bark Extract, Quillaja Extract (Quillaja Saponaria Molina), Quillaia (Quillaja Saponaria Molina), Quince Seed, Extract (Cydonia Spp.), Quinine Bisulfate, Quinine Hydrochloride, Quinine Sulfate, Quinoline, Rapeseed Oil, Hydrogenated, Rapeseed Oil, Hydrogenated, Superglycerinated, Rapeseed Oil, Low Erucic Acid, Rapeseed Oil, Low Erucic Acid, Partially Hydrogenated, Rebaudioside C., Rebaudioside A., Rebaudioside D., Rennet, Resin, From Formaldehyde, Acetone, And Tetraethylenepentamine, Resorcinol, L-Rhamnose, Rhatany, Extract (Krameria Spp.), Rhodinol, Rhodinyl Acetate, Rhodinyl Butyrate, Rhodinyl Formate, Rhodinyl Isobutyrate, Rhodinyl Isovalerate, Rhodinyl Phenylacetate, Rhodinyl Propionate, Rhubarb, Garden Root (Rheum Rhaponticum L.), Rhubarb Root (Rheum Spp.), Riboflavin, Riboflavin 5′-Phosphate, Riboflavin 5′-Phosphate, Sodium, D-Ribose, Rice Bran Wax, Rice, Milled, Rice Starch, (R)-(−)1-Octen-3-O1, Rose, Absolute (Rosa Spp.), Rose, Bud (Rosa Spp.), Rose Flowers (Rosa Spp.), Rose Hips, Extract (Rosa Spp.), Rose Leaves (Rosa Spp.), Roselle (Hibiscus Sabdariffa L.), Rosemary, Extract (Rosmarinus Officinalis L.), Rosemary, Oil (Rosemarinus Officinalis L.), Rosemary, Oleoresin, Rosemary (Rosemarinus Officinalis L.), Rose, Oil (Rosa Spp.), Rose Water, Stronger (Rosa Centifolia L.) Rosidinha (Micropholis (Also Known As Sideroxylon) Spp.), Rosin, Adduct With Fumaric Acid, Pentaerythritol Ester, Rosin, Glycerol Ester, Rosin, Gum, Glycerol Ester, Rosin, Gum Or Wood, Partially Hydrogenated, Glycerol Ester, Rosin, Gum Or Wood, Partially Hydrogenated, Pentaerythritol Ester, Rosin, Gum Or Wood, Pentaerythritol Ester, Rosin, Limed, Rosin, Methyl Ester, Partially Hydrogenated, Rosin, Partially Dimerized, Calcium Salt, Rosin, Partially Dimerized, Glycerol Ester, Rosin, Partially Hydrogenated, Rosin (Pinus Spp.) And Rosin Derivatives, Rosin, Polymerized, Glycerol Ester, Rosin, Tall Oil, Glycerol Ester, Rosin, Wood, Rosin, Wood, Glycerol Ester, Rosin, Wood, Maleic Anhyd. Mod., Pentaerythritol Ester, Acid #176-186, Rosin, Wood, Maleic Anhyd. Mod., Pentaerythritol Ester, Acid #134-145, (1r,2s,5r)-N-(4-Methoxypbenyl)-5-Methyl-2-(1-Methylethyl)Cyclohexanecarboxamide, Rubber, Natural-Smoked Sheet And Latex Solids (Hevea Brasiliensis), Rue, Oil (Ruta Graveolens L.), Rue (Ruta Graveolens L.), Rum, Rum Ether, Rutin, Saccharin, Saccharin, Ammonium Salt, Saccharin, Calcium Salt, Saccharin, Sodium Salt, Saffron (Crocus Sativus L.), Saffron, Extract (Crocus Sativus L.), Safrole-Free Extract Of Sassafras, Sage, Greek (Salvia Triloba L.), Sage, Oil (Salvia Officinalis L.), Sage, Oleoresin (Salvia Officinalis L.), Sage (Salvia Officinalis L.), Sage, Spanish, Oil (Salvia Lavandulaefolia Vahl.), Salicylaldehyde, Salicylic Acid, Salts Of Fatty Acids, Sandalwood, Red (Pterocarpus Santalinus L.F.), Sandalwood, White (Santalum Album L.), Sandalwood, Yellow, Oil (Santalum Album L.), Sandarac (Tetraclinis Articulata (Vahl.) Mast.), Santalol, Beta, Santalol, Alpha, Santalol (Alpha And Beta), Santalyl Acetate, Santalyl Phenylacetate, Sarcodactylis Oil, Sarsaparilla, Extract (Smilax Spp.), Sassafras Bark, Extract (Safrole-Free) (Sassafras Albidum (Nutt.) Nees), Sassafras Leaves (Safrole-Free) (Sassafras Albidum (Nutt.) Nees), Sausage Casing (Hel And Cellulose Fibers), Savory, Summer, Oil (Satureja Hortensis L.), Savory, Summer, Oleoresin (Satureja Hortensis L.), Savory, Summer (Satureja Hortensis L.), Savory, Winter, Oil (Satureja Montana L.), Savory, Winter, Oleoresin (Satureja Montana L.), Savory, Winter (Satureja Montana L.), Schinus Molle, Oil (Schinus Molle L.), (−)-Sclareol, Sclareolide, Scotch Spearmint Oil, Mentha Cardiaca L., 2-Sec-Butylcyclohexanone, Sec-Butyl Ethyl Ether, Senna, Alexandria (Cassia Acutifolia Defile), L-Serine, Serpentaria (Aristolochia Serpentaria L.), Sesame (Sesamum Indicum L.), Sheanut Oil, Shellac, Purified, Shellac Wax, Silica Aerogel, Silicon Dioxide, Silver Fir, Needles And Twigs, Oil (Abies Alba Mill.), Silver-Silver Dragees, Simaruba Bark (Simaruba Amara Aubl.), Skatole, Sloe Berries, Extract (Prunus Spinosa L.), Sloe Berries, Extract Solid (Prunus Spinosa L.), Sloe Berries (Prunus Spinosa L.), Snakeroot, Canadian, Oil (Asarum Canadense L.), Sodium Acetate, Sodium Acid Pyrophosphate, Sodium N-Alkylbenzenesulfonate, Sodium Aluminate, Sodium Aluminum Phosphate, Acidic Or Basic, Sodium Aluminum Silicate, Sodium Ascorbate, Sodium Benzoate, Sodium Bicarbonate, Sodium Bisulfite, Sodium Borate, Sodium Borohydride, Sodium Calcium Aluminosilicate, Hydrated, Sodium Caprate, Sodium Caprylate, Sodium Carbonate, Sodium Caseinate, Sodium Chloride, Sodium Chlorite, Sodium Copper Chlorophyllin, Sodium Decylbenzenesulfonate, Sodium Dehydroacetate, Sodium Diacetate, Sodium Dimethyldithiocarbamate, Sodium Dodecylbenzenesulfonate, Sodium Erythorbate, Sodium 2-Ethylhexyl Sulfate, Sodium Ferricitropyrophosphate, Sodium Ferritripolyphosphate, Sodium Fluoride, Sodium Formate, Sodium Fumarate, Sodium Glucoheptonate, Sodium Gluconate, Sodium Hexametaphosphate, Sodium Humate, Sodium Hydrosulfite, Sodium Hydroxide, Sodium Hypochlorite, Sodium Hypophosphite, Sodium Lactate, Sodium Laurate, Sodium Lauryl Sulfate, Sodium 3-Mercaptooxopropionate, Sodium Metabisulfite, Sodium Metaphosphate, Sodium Metasilicate, Sodium (4-Methoxybenzoyloxy)Acetate, Sodium 3-Methoxy-4-Hydroxycinnamate, Sodium 2-(4-Methoxyphenoxy)Propanoate, Sodium Methyl Sulfate, Sodium Mono- And Dimethyl Naphthalene Sulfonates, Sodium Myristate, Sodium Nitrate, Sodium Nitrite, Sodium Oleate, Sodium Palmitate, Sodium Pantothenate, Sodium Pectinate, Sodium Phosphate, Dibasic, Sodium Phosphate, Monobasic, Sodium Phosphate, Tribasic, Sodium Polymethacrylate, Sodium Potassium Tartrate, Sodium Propionate, Sodium Pyrophosphate, Sodium Salts Of Fatty Acids, Sodium Sesquicarbonate, Sodium Silicate, Sodium Sorbate, Sodium Stearate, Sodium Stearoyl-2-Lactylate, Sodium Stearyl Fumarate, Sodium Sulfate, Sodium Sulfide, Sodium Sulfite, Sodium Tartrate, Sodium Taurocholate, Sodium Thiosulfate, Sodium Tripolyphosphate, Sodium Zinc Metasilicate, Sorbic Acid, Sorbitan Monooleate, Sorbitan Monostearate, D-Sorbitol, Sorbose, Soya Bean Oil Fatty Acids, Hydroxylated, Soya Fatty Acid Amine, Ethoxylated, Soybean Oil, Epoxidized, Soybean Oil, Hydrogenated, Soy Protein Concentrate, Enzyme Activated, Soy Protein, isolate, Spearmint, Extract (Mentha Spicata L.), Spearmint (Mentha Spicata L.), Spearmint, Oil (Mentha Spicata L.), Sperm Oil, Sperm Oil, Hydrogenated, Spikenard Extract, Spiro(2,4-Dithia-1-Methyl -8-Oxabicyclo(3.3.0)Octane-3,3′-(1′-Oxa-2′-Methyl)Cyclopentane), Spruce Needles And Twigs, Extract (Picea Spp.), Spruce Needles And Twigs, Oil (Picea Spp.), (2s,5r)-N-[4-(2-Amino-2-Oxoethyl)Phenyl]-5-Methyl-2-(Propan-2-Y1)Cyclohexanecarboxamide, Stannic Chloride, Stannous Chloride, Starch, Acid Modified, Starch, Alpha-Amylase Modified, Starch, Bleached, Starch, Food, Modified, Starch, Food, Modified: Acetylated Distarch Adipate, Starch, Food, Modified: Acetylated Distarch Glycerol, Starch, Food, Modified: Acetylated Distarch Oxypropanol, Starch, Food, Modified: Acetylated Distarch Phosphate, Starch, Food, Modified: Beta-Amylase Modified Starch, Starch, Food, Modified: Beta-Amylase Sodium Starch Octenylsuccinate, Starch, Food, Modified: Distarch Glycerol, Starch, Food, Modified: Distarch Oxypropanol, Starch, Food, Modified: Distarch Phosphate (From Phosphorus Oxychloride), Starch, Food, Modified: Distarch Phosphate (From Sodium Trimetaphosphate), Starch, Food, Modifid: Glucoamylase Modified Starch, Starch, Food, Modified: Hydroxypropyl Distarch Glycerol, Starch, Food, Modified: Hydroxypropyl Distarch Phosphate, Starch, Food, Modified: Hydroxypropyl Starch, Starch, Food, Modified: Isoamylase Modified. Starch, Starch, Food, Modified: Oxidized Hydroxypropyl Starch, Starch, Food, Modified: Oxidized Starches, Starch, Food, Modified: Phosphated Distarch Phosphate, Starch, Food, Modified: Pullulanase Modified Starch, Starch, Food, Modified: Starch Acetate, Starch, Food, Modified: Starch Aluminum Octenyl Succinate, Starch, Food, Modified: Starch Phosphate, Starch, Food, Modified: Starch Sodium Octenyl Succinate, Starch, Food, Modified: Starch Sodium Succinate, Starch, Food, Modified: Succinyl Distarch Glycerol, Starch, Pregelatinized, Starch, Unmodified, Stearic Acid, Stearyl Alcohol, Stearyl Alcohol, Plus Beeswax, Stearyl Citrate, Stearyl Monoglyceridyl Citrate, S-(Tetrahydro-2,5-Dimethyl-3-Furanyl) Ethanethioate, St. Johnswort Leaves, Flowers And Caulis (Hypericum Perforatuml.), Storax Extract (Liquidambar Spp.), Storax (Liquidambar Spp.), Storax Oil, Styrene, Styrene-Divinylbenzene-Acrylonitrile, Sulfonated Terpolymer, Styrene-Divinylbenzene Copolymer, Chloromethylated, Aminated, Oxidized, Styrene-Divinylbenzene-Methyl Acrylate,Sulfonated Terpolymer, Styrene, Divinylbenzene, Sulfonated Copolymer, Styrene-Dvb-Acrylonitrile-Methyl Acrylate, Sulfonated Tetrapolymer, Succinic Acid, Succinic Anhydride, Succinylated Gelatin, Succinylated Monoglycerides, Succistearin, Sucralose, Sucrose, Sucrose Acetate Isobutyrate, Sucrose Fatty Acid Esters, Sucrose Liquid, Sucrose Monopalmitate, Sucrose Octaacetate, Sucrose Oligoesters, Sugar Beet Juice Extract, Sugar Beet Extract Flavor Base, Sulfamic Acid, Sulfites, Strong Alkali, Suifiting Agents, Sulfopropyl Cellulose, Sulfur Dioxide, Sulfuric Acid, Sulfurous Acid, Sweet Blackberry Leaves Extract, Tagetes Meal & Extract, Tagetes, Oil (Tagetes Spp.), Talc, Tallow Alcohol, Hydrogenated, Tallow, Beef, Tallow Flakes, Tallow, Hydrogenated, Tallow, Hydrogenated, Oxidized Or Sulfated, Tamarind Extract (Tamarindus indica L.), Tamarinds, Tangerine, Essence, Tangerine, Extract (Citrus Reticulata Blanco), Tangerine, Oil (Citrus Reticulata Blanco), Tannic Acid, Tansy, Oil (Tanacetum Vulgara L.), Tansy (Tanacetum Vulgara L.), Tapioca Starch, Tarragon (Artemisia Dracunculus L.), Tarragon Extract (Artemisia Dracunculus L.), Tarragon Oil (Artemisia Dracunculus L.), Tartaric Acid, L, Taurine, Taurocholic Acid, Tea Extract (Thea Sinensis L.), Tea Tree Oil (Melaleuca Alternifolia), Terpene Resin, Terpene Resins, Natural, Terpene Resins, Synthetic, Alpha-Terpinene, Gamma-Terpinene, Alpha-Terpineol, Beta-Terpineol, Terpinolene, Acetate, Alpha-Terpinyl Anthranilate, Terpinyl Butyrate, Terpinyl Cinnamate, Terpinyl Formate, Terpinyl Isobutyrate, Terpinyl Isovalerate, Terpinyl Propionate, Tert-Butylhydroquinone, 2-Tert-Pentylcyclohexyl Acetate, Delta-Tetradecalactone, (Z)-8-Tetradecenal, Tetradec-2-Enal, 9-Tetradecen-5-Olide, Tetraethylenepentamine Crosslinked With Epichlorohydrin, 1,1′-(Tetrahydro-6a-Hydroxy-2,3a,5-Trimethylfuro[2,3-D]-1,3-Dioxole-2,5-Diyl)Bis-Ethanone, 1,2,5,6-Tetrahydrocuminic Acid, 4,5,6,7-Tetrahydro-3,6-Dimethylbenzofuran, Tetrahydrofurfuryl Acetate, Tetrahydrofurfuryl Alcohol, Tetrahydrofurfuryl Butyrate, Tetrahydrofurfuryl Cinnamate, 2-Tetrahydrofurfuryl 2-Mercaptopropionate, Tetrahydrofurfuryl Propionate, Tetrahydrolinalool, Tetrahydro-4-Methyl-2-(2-Methylpropen-1-Y1)Pyran, Tetrahydro-Pseudo-Ionone, 5,6,7,8-Tetrahydroquinoxaline, 2,2,6,7-Tetramethylbicyclo[4.3.0]Nona-4,9(1)-Dien-8-O1, 2,2,6,7-Tetramethylbicyclo[4.3.0]Nona-4,9(1)Dien-8-One, Alpha-(P-(1,1,3,3-Tetramethylbutyl)Phenyl)-Omega-Hydroxypoly(Oxyethylene) (1 Mol), Alpha-{P-(1,1,3,3-Tetramethylbutyl)Phenyl}-Omega-Hydroxypoly(Oxyethylene), Alpha-(P-(1,1,3,3-Tetramethylbutyl)Phenyl)-Omega-Hydroxypoly(Oxyethylene) (Greater Than 1 Mol), Tetramethyl Ethylcyclohexenone (Mixture Of Isomers), (+/−)-2,6,10,10-Tetramethyl-1-Oxaspiro[4,5]Deca-2,6-Dien-8-One, 1,5,5,9-Tetramethyl-13-Oxatricyclo(8.3.0.0(4,9))Tridecane, 2,3,5,6-Tetramethylpyrazine, Thaumatin, Thaumatin B, Recombinant, Theaspirane, Theobromine, Thiamine, Thiamine Hydrochloride, Thiamine Mononitrate, Thiazole, 2-Thienyl Disulfide, 1-(2-Thienyl)Ethanethiol, 2-Thienyl Mercaptan, 2-Thienylmethanol, Thioacetic Acid, 2,2′-(Thiodimethylene)-Difuran, Thiodipropionic Acid, Thiogeraniol, Thistle, Blessed (Cnicus Benedictus L.), Thistle, Blessed, Extract (Cnicus Benedictus L.), Thistle, Blessed, Extract Solid (Cnicus Benedictus L.), Thistle, Blessed, Oil (Cnicus Benedictus L.), L-Threonine, 4-Thujanol, Thujyl Alcohol, Thyme, Extract, Thyme Oil (Thymus Vulgaris L. And T. Zygis Var. Gracilis Boiss,), Thyme Oleoresin, Thyme (Thymus Serpyllum L.), Thyme (Thymus Vulgaris L.), Thyme, Wild. Or Creeping, Extract (Thymus Serpyllum L.), Thymol, Titanium Dioxide, Alpha-Tocopherol Acetate, Tocopherols, Tolualdehyde Glyceryl Acetal (Mixed O-, M-, P-), Tolualdehydes (Mixed O-, M-, P-), Tolu Balsam Extract (Myroxylon Spp.), Tolu Balsam Gum (Myroxylon Spp.), O-Toluenethiol, P-Tolylacetaldehyde, O-Tolyl Acetate, P-Tolyl Acetate, 4-(P-Tolyl)-2-Butanone, O-Tolyl Isobutyrate, P-Tolyl Isobutyrate, P-Tolyl Laurate, P-Tolyl 3-Methylbutyrate, P-Tolyl Octanoate, P-Tolyl Phenylacetate, 2-(P-Tolyl)-Propionaldehyde, O-Tolyl Salicylate, Tomato Lycopene, Tragacanth, Gum (Astragalus Spp.), (+/−)-Trans- And Cis-2-Hexenal Propylene Glycol Acetal, 2-Trans-6-Cis-Dodecadienal, 2-Trans-6-Cis-Nonadienal, O-Trans-Coumaric Acid, Trans-2-Decenol, Trans-3-Heptenyl Acetate, Trans-3-Hexenol, Trans-3-Hexenyl Acetate, Trans-2-Hexen-1-Y1 Acetate, Trans-2-Hexenyl 2-Methlbutyrate, Trans-4-Nonenal, Trans-3-Nonen-1-O1, Trans-2-Nonen-4-One, Trans-2-Nonenyl Acetate, Trans-4-Octenoic Acid, 2-(Trans-2-Pentenyl)Cyclopentanone, Trans-4-Tert-Butylcyclohexanol, Trans,Trans-2,4-Dodecadienal, Trans-2-Trans-4-Nonadiene, Trans-2-Tridecenol, Trefoil, Sweet (Melilotus Coerulea), Trehalose, Dihydrate, Triacetin (Glycerol Triacetate), Tributyl Acetylcitrate, Trichloroethylene, Tridecanal, Tridecanoic Acid, 2-Tridecanone, 2-Trans,4-Cis,7-Cis-Tridecatrienal, 2-Tridecenal, Tridodecyl Amine, Triethanolamine, Triethylamine, Triethyl Citrate, Triethylenetetramine Cross-Linked With Epichlorohydrin, Trifluoromethane Sulfonic Acid, 2,4,5-Trihydroxybutyrophenone, (Trihydroxy-Phenyl)-Propan-1-One, Trilobatin, Trimethylamine, Trimethylamine Oxide, 2,6,6-Trimethyl-1 And 2-Cyclohexen-1-Carboxaldehyde, P,Alpha,Alpha-Trimethylbenzyl Alcohol, 4-(2,6,6-Trimethylcyclohexa-1,3-Dienyl)But-2-En-4-One, 2,6,6-Trimethylcyclohexa-1,3-Dienyl Methanal, 3,3,5-Trimethylcyclohexanol, 2,2,6-Trimethylcyclohexanone, 2,6,6-Trimethyl-1-Cyclohexen-1-Acetaldehyde, 2,6,6-Trimethylcyclohex-2-Ene-1,4-Dione, 4-(2,6,6-Trimethylcyclohex-1-Enyl)But-2-En-4-One, 3,3,5-Trimethylcyclohexyl Acetate, 2,2,3-Trimethylcyclopent-3-En-1-Y1 Acetaldehyde, 4-(2,2,3-Trimethylcyclopentyl)Butanoic Acid, 3,7,11-Trimethyldodeca-2,6,10-Trienyl Acetate, 3,5,5-Trimethylhexanal, 3,5,5-Trimethyl-1-Hexanol, 2,6,6-Trimethyl -2-Hydroxycyclohexanone, (2,6,6-Trimethyl-2-Hydroxycyclohexylidene)Acetic Acid Gamma-Lactone, 2,3,3-Trimethylindanone, Trans- And Cis-2,4,8-Trimethyl-3,7-Nona-Dien-2-O1, (+/−2,4,8-Trimethyl-7-Nonen-2-O1, 1,3,3-Trimethyl-2-Norbornanyl Acetate, 2,2,4-Trimethyl-1,3-Oxacyclopentane, Trimethyloxazole, 2,4,5-Trimethyl-Delta-3-Oxazotine, 2,6,10-Trimethyl-2,6,10-Pentadecatrien-14-One, 2,3,4-Trimethyl-3-Pentanol, 2,4,6-Trimethylphenol, 2,3,6-Trimethylphenol, 2,3,5-Trimethylpyrazine, 2,4,5-Trimethylthiazole, 2,2,6-Trimethyl-6-Vinyltetrahydropyran, Tripropylamine, 1,2,3-Tris((1′-Ethoxy)Ethoxy)-Propane, Trisodium Citrate, Trisodium Nitrilotriacetate, 2,4,6-Trithiaheptane, Trithioacetone, Trypsin From Animal Tissue, L-Tryptophan, Tuberose Lactone, Tuberose, Oil (Polianthes Tuberosa L.) Tunu (Castilla flax Cook)), Turmeric (Curcuma Longa L.), Turmeric Extract (Curcuma Longa L.), Turmeric Oleoresin (Curcuma Longa L.), Turpentine, Turpentine, Gum (Pinus Spp.), Turpentine, Rectified, Turpentine, Steam Distilled (Pinus Spp.), Tyramine, L-Tyrosine, L-Tyrosine Ethyl Ester Hydrochloride, Ultramarine Blue, 2,4-Undecadienal, 2,5-Undecadienal, 2,3-Undecadione, Gamma-Undecalactone, Undecanal, Undecanal Propyleneglycol Acetal, Undecanoic Acid, 2-Undecanol, 2-Undecanone, 1,3,5,7-Undecatetraene, 1,3,5-Undecatriene, 9-Undecenal, 10-Undecenal, 2-Undecenal, (E)-4-Undecenal, 10-Undecenoic Acid, 2-Undecen-1-O1, Undecen-1-O1, 2-Undecen-1-O1, 10-Undecen-2-One, 10-Undecen-1-Y1 Acetate, Undecyl Alcohol, N-Undecylbenzenesulfonic Acid, Urea, Urease Enzyme Preparation (Lactobacillus Fermentum), Valencene, Valeraldehyde, Valeraldehyde Dibutyl Acetal, Valeraldehyde Propyleneglycol Acetal, Valerian Root, Extract (Valeriana Officinalis L.), Valerian Root, Oil (Valeriana Officinalis L.), Valerie Acid, Gamma-Vaterolactone, L-Valine, Vanilla, Absolute (Vanilla Spp.), Vanilla, Extract (Vanilla Spp.), Vanilla, Oleoresin (Vanilla Spp.), Vanilla (Vanilla Spp.), Vanillic Acid, Vanillin, Vanillin Acetate, Vanillin 1,2-Butylene Glycol Acetal, Vanillin Isobutyrate, Vanillin 3-(L-Menthoxy)Propane-1,2-Diol Acetal, Vanillin Propylene Glycol Acetal, Vanillyl Alcohol, Vanillyl Butyl Ether, Vanillyl Ethyl Ether, Vanillylidene Acetone, Vegetable Gum, Other Than Those Cfr Listed, Vegetable Juice, Veratraldehyde, Verbenol, Verbenone, Veronica (Veronica Officinalis L.), Vervain, European (Verbena Officinalis L.), Vetiver, Oil (Vetiveria Zizanioides Stapf), Vetiverol, Vetiver (Vetiveria Zizaniodes Stapt), Vetiveryl Acetate, Vinyl Acetate, O-Vinylanisole, Vinyl Chloride-Vinylidene Chloride Copolymer, P-Vinylphenol, Violet Leaves Absolute (Viola Odorata L.), Violet, Swiss (Viola Calcarata L.), Vitamin D-2, Vitamin D-3, Vitamin B-12, Vitamin A, Vitamin A Acetate, Vitamin B Complex And. Syrup, Vitamin D, Vitamin K, Vitamin A Palmitate, Walnut Hull, Extract (Juglans Spp.), Walnut Leaves, Extract (Juglans Spp.), Wheat Gluten, Wheat Starch, Whey, Whey, Delactosed, Whey, Demineralized, Whey, Partially Dimineralized And Partially Delactosed, Whey Protein Concentrate, Whey Powder, Wintergreen, Extract (Gaultheria Procumbens L.), Wintergreen, Oil (Gaultheria Procumbens L.), Woodruff, Sweet (Asperula Odorata L.), Wort, Xanthan Gum, Xanthophyll, 2,6-Xylenol, 2,5-Xylenol, 3,4-Xylenol, Xylitol, D-Xylose, Yarrow, Herb (Achillea Millefolium L.), Yarrow, Oil (Achillea Millefolium L.), Yeast Autolysate, Yeast, Dried Irradiated, Yeast Extract Autolyzed, Yeast-Malt Sprout Extract, Yeasts, Dried Yeasts, Yellow Prussiate Of Soda, Yerba Santa, Fluid Extract (Eriodictyon Californicum (Hook And Am) Torr), Ylang-Ylang, Oil (Cananga Odorata Hook. F. And Thomas), Yucca, Joshua-Tree (Yucca Brevifolia Engelm.), Yucca, Mohave, Extract (Yucca Spp.), Yuzunone, Zedoary Bark, Extract (Curcuma Zedoaria (Berg.) Rosc.), Zedoary (Curcuma Zedoaria (Berg.) Rosc.), Zein Powder, Zinc Acetate, Zinc Carbonate, Zinc Chloride, Zinc Dithionite, Zinc Gluconate, Zinc Methionine Sulfate, Zinc Oxide, Zinc Stearate, Zinc Sulfate, and Zingerone.

Food additives may also include, without limitation, vanillin, ethyl vanillin, 2-hydroxy-4-methoxybenzaldehyde, ethyl vanillin isobutyrate (=3 ethoxy-4-isobutyryloxybenzaldehyde), Furaneol.®. (2,5-dimethyl-4-hydroxy-3(2H)-furanone) and derivatives (e.g. homofuraneol, 2-ethyl-4-hydroxy-5-methyl-3(2H)-furanone), homofuronol (2-ethyl-5-methyl-4-hydroxy-3(2H)-furanone and 5-ethyl-2-methyl-4-hydroxy-3(2H)-furanone), maltol and its derivatives (e.g. ethyl maltol), coumarin and its derivatives, gamma-lactones (e.g. gamma-undecalactone, gamma-nonalactone), delta-lactones (e.g. 4-methyl delta-lactone, massoia intone, deltadecalactone, tuberolactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, fruit esters and fruit lactones (e.g. acetic acid n-butyl ester, acetic acid isoamyl ester, propionic acid ethyl ester, butyric acid ethyl ester, butyric acid n-butyl ester, butyric acid isoamyl ester, 3-methyl-butyric acid ethyl ester, n-hexanoic acid ethyl ester, n-hexanoic acid allyl ester, n-hexanoic acid n-butyl ester, n-octanoic acid ethyl ester, ethyl-3-methyl-3-phenylglycidate, ethyl-2-trans-4-cis-decadienoate), 4-(p-hydroxyphenyl)-2-butanone, 1,1-ditnethoxy-2,2,5-trimethyl-4-hexane, 2,6-dimethyl-5-hepten-1-al, and phenylacetaldehyde and mixtures thereof; (a2) carbohydrates selected from the group consisting of saccharose, trehalose, lactose, maltose, melizitose, melibiose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, maltodextrins and mixtures thereof and plant formulations containing one or a plurality of the cited carbohydrates, preferably in a proportion of at least 5 wt %, preferably at least 15 wt %, whereby the carbohydrates can also be present as a naturally occurring or synthetically produced mixture, in this arrangement particularly as honey, invert sugar syrup or highly enriched fructose syrup from maize starch, and the physiologically acceptable salts of these carbohydrates, particularly sodium, potassium, calcium or ammonium salts; (a3) sugar alcohols, preferably naturally occurring sugar alcohols selected from the group consisting of glycerine, erythritol, threitol, arabitol, ribitol, xylitol, sorbitol, mannitol, maltitol, isomalt, dulcitol, lactitol and mixtures thereof, and the physiologically acceptable salts of these sugar alcohols, particularly sodium, potassium, calcium or ammonium salts; (a4) naturally occurring sweeteners, preferably selected from the group consisting of (a4-1)miraculin, monellin, mabinlin, thaumatin, curculin, brazzein, pentaidin, D-phenylalanine, D-tryptophan, and extracts or fractions obtained from natural sources containing these amino acids and/or proteins and mixtures thereof, and the physiologically acceptable salts of these amino acids and/or proteins, particularly the sodium, potassium, calcium or ammonium salts; (a4-2)neohesperidin dihydrochalcone, naringin dihydrochalcone, stevioside, steviolbioside, rebaudiosides, particularly rebaudioside A, rebaudioside B, rebaudioside C, rebaudioside D, rebaudioside E, rebaudioside F, rebaudioside G, rebaudioside H, dulcosides and rubusoside, suavioside A, suavioside B, suavioside G, suavioside H, suavioside I, suavioside J, baiyunoside 1 baiyunoside 2, phlomisoside 1, phlomisoside 2, phlomisoside 3, and phlomisoside 4, abrusoside A, abrusoside B, abrusoside C, abrusoside D, cyclocaryoside A and cyclocaryoside I, osladin, polypodoside A, strogin 1, strogin 2, strogin 4, selligueain A, dihydroquercetin-3-acetate, perillartine, telosmoside A.sub.15, periandrin I-V, pterocaryosides, cyclocaryosides, mukuroziosides, trans-anethole, trans-cinnamaldehyde, bryosides, bryonosides, bryonodulcosides, carnosiflosides, scandenosides, gypenosides, trilobatin, phloridzin, dihydroflavanols, hematoxylin, cyanine, chlorogenic acid, albiziasaponin, telosmosides, gaudichaudioside, mogrosides, mogroside V, hernandulcins, monatin, phyllodulcin, glycyrrhetinic acid, and derivatives thereof and mixtures thereof, particularly its glycosides such as glycyrrhizin, and the physiologically acceptable salts of these compounds, particularly the sodium, potassium, calcium or ammonium salts; (a4-3) extracts or enriched fractions of extracts, selected from the group consisting of Thaumatococcus extracts (katemfe bush), extracts of Stevia ssp. (particularly Stevia rebaudiana), swingle extracts (Momordica or Siratia grosvenorii, Luo-Han-Guo), extracts of Glycerrhyzia ssp. (particularly Glycerrhyzia glabra), extracts of Rubus ssp. (particularly Rubus suavissimus), citrus extracts, and extracts of Lippia dulcis and mixtures thereof, and mixtures of any of (a4-1) to (a4-3); (a5) synthetically sweet-tasting substances, preferably selected from the group consisting of magap, sodium cyclamate or other physiologically acceptable salts of cyclamic acid, acesulfame K or other physiologically acceptable salts, neohesperidin dihydrochalcone, naringin dihydrochalcone, saccharin, saccharin-sodium salt, aspartame, superaspartame, neotame, alitame, advantame, perillartine, sucralose, lugduname, carrelame, sucrononate, and sucrooctate.

In some embodiments, the combination of the umami flavor compound or umami flavor enhancing compounds with the food additives described above has a synergistic effect. For example, the combination may further enhance the umami flavor of the composition or enhance the properties of the food additive.

### Flavorants

In some embodiments, the composition comprises an umami flavor compound or umami flavor enhancing compound and one or more flavorants. Representative flavorants include but are not limited to, those compounds identified as such by the Flavor and Extract Manufacturers Association (FEMA; see the FEMA Flavor Ingredient Library, accessible at http://www.femaflavor.org/flavor, last accessed Nov. 16, 2015). Such flavorants include: Acacia Gum (Acacia Senegal (L.) Willd.), Acetal, Acetaldehyde, Propyl Phenethyl Acetal, Acetanisole, Acetic Acid, (Tri-)Acetin, Acetoin, Acetophenone, Aconitic Acid, Adipic Acid, Agar (Gelidium Spp.), Alfalfa Extract (Medicago Sativa L.), Algin (Laminaria Spp. And Other Kelps), Alginates, Sodium, Calcium, And Ammonium, Allspice (Pimenta Officinalis Lindl.) Allspice Oil (Pimenta Officinalis Lindl.), Allspice Oleoresin (Pimenta Officinalis Lindl.), Allyl Anthranilate, Allyl Butyrate, Allyl Cinnamate, Allyl Cyclohexaneacetate, Allyl Cyclohexanebutyrate, Allyl Cyclohexanehexanoate, Allyl Cyclohexanepropionate, Allyl Cyclohexanevalerate, Allyl Disulfide, Allyl 2-Ethylbutyrate, Allyl 2-Furoate, Allyl Heptanoate, Allyl Hexanoate, Allyl A Alpha-Ionone, Allyl Isothiocyanate, Allyl Mercaptan, Allyl Nonanoate Alkyl Octanoate, Allyl Phenoxyacetate, Allyl Phenylacetate, Allyl Propionate, Allyl Sorbate, Allyl Sulfide, Allyl Tiglate, Allyl 10-Undecenoate, Ally Isovalerate, Almond Oil, Bitter (Ffpa) (Prunus Spp.), Aloe Extract (Aloe Spp.), Althea Root (Althea Officinalis L.), Ambergris Tincture, Ambrette Absolute Oil (Hibiscus Abelmoschus L.), Ambrette Seed Oil (Hibiscus Abelmoschus L.) Ambrette Tincture (Hibiscus Abelmoschus L.), Ammonium Sulfide, Ammonium Isovalerate, Isoamyl Acetate, Amyl Alcohol, Isoamyl Alcohol, Isoamyl Benzoate, Amyl Butyrate, Isoamyl Butyrate, Alpha-Amyicinnamaldehyde, Alpha-Amylcinnamaldehyde Dimethyl Acetal, Isoamyl Cinnamate, Alpha-Amylcinnamyl Acetate, Alpha-Amylcinnamyl Alcohol, Alpha-Amylcinnamyl Formate, Alpha-Amylcinnamyl Isovalerate, Amyl Formate, Isoamyl Formate, Isoamyl 4(2-Furan)Butyrate, Isoamyl 3(2-Furan)Propionate, Amyl 2-Furoate, Amyl Heptanoate, Amyl Hexanoate, Isoamyl Hexanoate, 2-Amyl-5 Or 6-Keto-1,4-Dioxane, Isoamyl Laurate, Isoamyl Nonanoate, Amyl Octanoate, Isoamyl Octanoate, Isoamyl Phenylacetate, Isoamyl Propionate, Isoamyl Pyruvate, Isoamyl Salicylate, Isoamyl Isovalerate, Trans-Anethole, Angelica Root Extract (Angelica Archangelica L.), Angelica Root Oil (Angelica Archangelica L.), Angelica Seed Extract (Angelica Archangelica L.), Angelica Seed Oil (Angelica Archangelica L.), Angelica Stem Oil (Angelica Archangelica L.), Angostura Extract (Galipea Offincinalis Hancock), Anise (Pimpinella Anisum L.), Anise Oil (Pimpinella Anisum L), Anise, Star (Illicium Verum Hook, F.), Anise, Star, Oil (Illicium Verum Hook, F.), Anisole, P-Anisyl Acetate, Anisyl Alcohol, Anisyl Butyrate, Anisyl Formate, Anisyl Propionate, Annatto Extract (Bixa Orellana L.), Annatto Seed (Bixa Orellana L.), Apricot Kernel Oil (Prunus Armeniaca L.), Asafetida Fluid Extract (Ferula Assafoetida L.), Asafetida Gum (Ferula Assafoetida L.), Asafoetida Oil (Ferula Asafoetida L.), Ascorbic Acid, Ash Bark, Prickly, Extract (Xanthoxylum Spp.), Balm (Melissa Officinalis L.), Bairn Leaves Extract (Melissa Officinalis Balm Oil (Melissa Officinalis L.), Balsam Fir Oil Abies Balsamea (L.) Balsam Fir Oleoresin (Abies Balsamea (L.) Mill.), Balsam, Peru (Myroxylon Pereirae Klotzsch), Balsam Oil, Peru (Myroxylon Pereirae Klotzsch), Basil (Ocimum Basilicum L.), Basil Oil (Ocimum Basilicum L.) Basil Oleoresin (Ocimum Basilicum L.), Bay Leaves, West Indian, Extract (Pimenta Acris Kostel), Bay Leaves, West Indian, Oil (Pimenta Acris Kostel), Bay Leaves, West Indian, Oleoresin (Pimenta Acris Kostel), Bay, Sweet (Laurus Nobilis L.), Bay Oil, Sweet (Laurus Nobilis L.), Beeswax, White (Apis Mellifera L.), Benzaldehyde, Benzaldehyde Dimethyl Acetal, Benzaldehyde Glyceryl Acetal, Benzaldehyde Propylene Glycol Acetal, Benzoic Acid, Benzoin, Benzoin Resinoid, Benzophenone, Benzyl Acetate, Benzyl Acetoacetate, Benzyl Alcohol, Benzyl Benzoate, Benzyl Butyl Ether, Benzyl Butyrate, Benzyl Isobutyrate, Benzyl Cinnamate, Benzyl 2,3-Dimethylcrotonate, Benzyl Ethyl Ether, Benzyl Formate, 3-Benzyl-4-Heptanone, Benzyl Mercaptan, Benzyl Methoxyethyl Acetal, Benzyl Phenylacetate, Benzyl Propionate, Benzyl Salicylate, Benzyl Isovalerate, Bergamot Oil, Birch, Sweet, Oil (Betula Lenta L.), Blackberry Bark Extract (Rubus, Spp. Of Section Eubatus), Bois De Rose Oil, Borneol, Isoborneol, Bornyl Acetate, Isobornyl Acetate, Bornyl Formate, Isobornyl Formate, Isobornyl Propionate, Bornyl Valerate, Bornyl Isovalerate (Endo-), Isobornyl Isovalerate, Boronia Absolute (Boronia Megastignia Nees), Buchu Leaves Oil (Barosma Spp.), 2-Butanone, Butter Acids, Butter Esters, Butter Starter Distillate, Butyl Acetate, Isobutyl Acetate, Butyl Acetoacetate, Isobutyl Acetoacetate, Butyl Alcohol, Isobutyl Alcohol, Isobutyl Angelate, Butyl Anthranilate, Isobutyl Anthranilate, Butylated Hydroxyanisole, Butylated Hydroxytoluene, Isobutyl Benzoate, Butyl Butyrate, Isobutyl Butyrate, Butyl Isobutyrate, Isobutyl Isobutyrate, Butyl Butyryllactate, Alpha-Butylcinnamaldehyde, Butyl Cinnamate, Isobutyl Cinnamate, Butyl 2-Decenoate, Butyl Ethyl Malonate, Butyl Formate, Isobutyl Formate, Isobutyl 3-(2-Furan)Propionate, Butyl Heptanoate, Isobutyl Heptanoate, Butyl Hexanoate, Isobutyl Hexanoate, Butyl P-Hydroxy Benzoate, 2-Butyl.-5- Or 6-Keto-1,4-Dioxane, Butyl Lactate, Butyl Laurate, Butyl Levulinate, Alpha-Isobutylphenethyl Alcohol, Butyl Phenylacetate, Isobutyl Phenylacetate, Butyl Propionate, Isobutyl Propionate, Isobutyl Salicylate, Butyl Stearate, Butyl Sulfide, Butyl 10-Undecenoate, Butyl Valerate, Butyl Isovalerate, Butyraldehyde, Isobutyraldehyde, Butyric Acid, Isobutyric Acid, (Tri-)Butyrin, Caffeine, Cajeput Oil (Melaleuca Leucadendron L.), Calcium Acetate, Camphene, D-Camphor, Camphor, Japanese, White, Oil (Cinnamomum Camphora (L) Nees Et Eberm.), Cananga Oil, Capsicum Extract (Capsicum Spp.), Capsicum Oleoresin (Capsicum Spp.), Caramel Color, Caraway (Carum Carvi L.), Caraway, Black (Nigella Sativa L.), Caraway Oil, Carboxymethylcellulose, Cardamom (Elettaria Cardamomum (L.) Maton), Cardamom Seed Oil (Elettaria Cardamomum (L.) Maton), Carmine (Coccus Cacti L.), Carob Bean Extract (Ceratonia Siliqua L.), Carrot Oil, Carvacrol, Carvacryl Ethyl Ether, Carveol, 4-Carvomenthenol, Carvone, Carvyl Acetate, Carvyl Propionate, Beta-Caryophyllene, Cascara Bitterless Extract (Rhamnus Purshiana De), Cascarilla Bark Extract (Croton Spp.), Cascarilla Bark Oil (Croton Spp.), Cassia (Cinnamomum Cassia Blume), Cassia Bark Extract (Cinnamomum Cassia Blume), Cassia Bark Oil, Cassia Buds (Cinnamomum Cassia Blume), Cassie Absolute (Acacia Farnesiana (L.) Willd.) Castoreum Oil, Castoreum, liquid (Castor Spp.), Castor Oil (Ricinus Communis L.), Catechu Extract (Acacia Catechu Willd.) Catechu Powder (Acacia Catechu Willd.), Cayenne (Capsicum Annuum. L. Var. Longum Sendt), Cedar Leaf Oil (Thuja Occidentalis L.), Celery Seed (Apium Graveolens L.), Celery Seed Extract (Apium Graveolens L.), Celery Seed Extract Solid (Apium Graveolens L.), Celery Seed Oil, Chamomile Flower, English, Oil (Anthemis Nobilis L), Chamomile Flower, Hungarian, Oil (Matricaria Chamomilla L.), Chamomile Flower, Roman, Extract (Anthemis Nobilis L), Chamomile Flower, Roman, Oil (Anthemis Nobilis L.), Cherry Bark, Wild, Extract (Prunus Serotina Ehrh.), Cherry Laurel Oil (Ffpa) (Prunus Laurocerasus L.), Cherry Pits Extract (Prunus Spp.), Chervil (Anthriscus Cerefolium (L.) Hoffm.), Chicory Extract (Cichorium Intybus L.), Cinchona Bark Red (Cinchona Succirubra Pav. Or Its Hybrids), Cinchona Bark Red Extract (Cinchona Succirubra Pav. Or Its Hybrids), Cinchona Bark Yellow (Cinchona Spp.), Cinchona Bark Yellow Extract (Cinchona Spp.), Cinchona Extract (Cinchona Spp.), Cinnamaldehyde, Cinnamaldehyde Ethylene Glycol Acetal, Cinnamic Acid, Cinnamon (Cinnamomum Spp.), Cinnamon Bark Extract (Cinnamonium Spp.), Cinnamon Bark Oil, Cinnamon Leaf Oil, Cinnamyl Acetate, Cinnamyl Alcohol, Cinnamyl Butyrate, Cinnamyl Isobutyrate, Cinnamyl Cinnamate, Cinnamyl Formate, Cinnamyl Phenylacetate, Cinnamyl Propionate, Cinnamyl Isovalerate, Citral, Citral Diethyl Acetal, Citral Dimethyl Acetal, Citric Acid, Citronellal, Citronella Oil, D1-Citronellol, Citronelloxyacetaldehyde, Citronellyl Acetate, Citronellyl Butyrate, Citronellyl Isobutyrate, Citronellyl Formate, Citronellyl Phenylacetate, Citroneltyl Propionate, Citronellyl Valerate, Citrus Peels Extract (Citrus Spp.), Civet Absolute (Viverra Civetta Schreber And Viverra Zibetha Schreber), Clary (Salvia Sclarea L), Clary Oil (Salvia Sclarea L.), Clove Bud Extract (Eugenia Spp.), Clove Bud Oil (Eugenia Spp.), Clove Bud Oleoresin (Eugenia Spp.), Clove Leaf Oil, Madagascar, Clover Tops Red Extract Solid (Trifolium Pratense L.), Cloves (Eugenia Spp.), Clove Stem, Oil (Eugenia Spp.), Coca Leaf Extract (Decocainized) (Erythroxylon Coca Lam.), Cognac Oil, Green, Cognac Oil, White, Coriander (Coriandrum Sativum L.), Coriander Oil (Coriandrum Sativum L.), Corn Silk (Zea Mays L.), Costus Root Oil (Saussurea Lappa Clarke), P-Cresol, Cubebs (Piper Cubeba L. F.), Cubeb Oil (Piper Cubeba L. F.), Cumin (Cuminum Cyminum L.), Cuminaldehyde, Cumin Black (Nigella Sativa L.), Cumin Oil, Curacao Peel Extract (Citrus Aurantium L.), Curacao Peel Oil (Citrus Aurantium L.), Currant Buds Black Absolute (Ribes Nigrum L.), Cyclohexaneacetic Acid, Cyclohexaneethyl Acetate, Cyclohexyl Acetate, Cyclohexyl Anthranilate, Cyclohexyl Butyrate, Cyclohexyl Cinnamate, Cyclohexyl Formate, Cyclohexyl Propionate, Cyclohexyl Isovalerate, P-Cymene, Dandelion Fluid Extract (Taraxacum Spp.), Dandelion Root Extract Solid (Taraxacum Spp.), Davana Oil (Artemisia Pallens Wall.) Gamma-Decalactone, Delta-Decalactone, Decanal, Decanal Dimethyl Acetal, Decanoic Acid, 1-Decanol, 2-Decenal, Decyl Acetate, Decyl Butyrate, Decyl Propionate, Diacetyl, Dibenzyl Ether, 4,4-Dibutyl-Gamma-Butyrolactone, Dibutyl Sebacate, Diethyl Malate, Diethyl Malonate, Diethyl Sebacate, Diethyl Succinate, Diethyl Tartrate, Dihydrocarveol (Isomer Unspecified), Dihydrocarvyl Acetate, Dihydrocoumarin, Dill (Anethum Graveolens L.), Dill Oil (Anethum Graveolens L.), Dill Seed Indian (Anethum Spp.), M-Dimethoxybenzene, P-Dimethoxybenzene, 2,4-Dimethylacetophenone, Alpha,Alpha-Dimethylbenzyl Isobutyrate, 2,6-Dimethyl-5-Heptenal, 2,6-Dimethyloctanal, 3,7-Dimethyl-1-Octanol, Alpha,Alpha-Dimethylphenethyl Acetate, Alpha,Alpha-Dimethylphenethyl Alcohol, Alpha,Alpha-Dimethylphenethyl Butyrate, Alpha,Alpha-Dimethylphenethyl Formate, Dimethyl Succinate, 1,3-Diphenyl-2-Propanone, Disodium Phosphate, Dittany Of Crete (Origanum Dictamnus L.), Gamma-Dodecalactone, Delta-Dodecalactone, 2-Dodecenal, Doggrass Extract (Agropyron Repens (L.) Beauv.), Dragon's Blood Extract (Daemonorops Spp. Or Other Botanical Sources), Dulse (Rhodymenia Palmata (L.) Grev.), Elder Flowers (Sambucus Canadensis L. Or Sambucus Nigra L), Elemi Gum (Canarium Spp.), Elemi Oil (Canarium Spp.), Erigeron Oil (Erigeron Candensis L.), Erythrobic Acid, Estragole, Estragon Oil (Artemisia Dracunculus L.), P-Ethoxybenzaldehyde, Ethyl Acetate, Ethyl Acetoacetate, Ethyl 2-Acetyl-3-Phenylpropionate, Ethyl Aconitate (Mixed Esters), Ethyl Acrylate, Ethyl Alcohol, Ethyl P-Anisate, Ethyl Anthranilate, Ethyl Benzoate, Ethyl Benzoylacetate, Alpha-Ethylbenzyl Butyrate, 2-Ethylbutyl Acetate, 2-Ethylbutyraldehyde, Ethyl Butyrate, Ethyl Isobutyrate, 2-Ethylbutyric Acid, Ethyl Cinnamate, Ethyl Cyclohexanepropionate, Ethyl Decanoate, Ethyl Formate, Ethyl 3(2-Furyl)Propanoate, 4-Ethylguaiacol, Ethyl Heptanoate, 2-Ethyl-2-Heptenal, Ethyl Hexanoate, Ethyl Lactate, Ethyl Laurate, Ethyl Levulinate, Ethyl 2-Methylbutyrate, Ethyl Methylphenylglycidate, Ethyl Myristate, Ethyl Nitrite, Ethyl Nonanoate, Ethyl 2-Nonynoate, Ethyl Octanoate, Ethyl Oleate, Ethyl Palmitate, Ethyl Phenylacetate, Ethyl 4-Phenylbutyrate, Ethyl 3-Phenylglycidate, Ethyl 3-Phenylpropionate, Ethyl Propionate, Ethyl Pyruvate, Ethyl Salicylate, Ethyl Sorbate, Ethyl Tiglate, Ethyl 10-Undecenoate, Ethyl Valerate, Ethyl Isovalerate, Ethyl Vanillin, Eucalyptol, Eucalyptus Oil (Eucalyptus Globulus Labille). Eugenol, Isoeugenol, Eugenyl Acetate, Isoeugenyl Acetate, Eugenyl Benzoate, Isoeugenyl Ethyl Ether, Eugenyl Formate, Isoeugenyl Formate, Eugenyl Methyl Ether, Isoeugenyl Methyl Ether, Isoeugenyl Phenylacetate, Farnesol, D-Fenchone, Fenchyl Alcohol, Fennel, Common (Foeniculum Vulgare Mill), Fennel, Sweet (Foeniculum Vulgare Mill. Var Dulce (D.C.), Fennel Oil, Sweet (Foeniculum Vulgare Mill Var. Dulce Dc.), Fenugreek (Trigonella Foenum-Graecum L.), Fenugreek Extract (Trigonella Foenum-Graecum L.), Fenugreek Oleoresin (Trigonella Foenumgraecum L.), Formic Acid, Fumaric Acid, Furfural, Furfuryl Acetate, Furfuryl Alcohol, 2-Furfurylidene Butyraldehyde, Furfuryl Mercaptan, 3-(2-Furyl)Acrolein, 4-(2-Furyl)-3-Buten-2-One, (2-Furyl)-2-Propanone, Fusel Oil, Refined, Galangal Root (Alpinia Spp.), Galangal Root Extract (Alpinia Spp.), Galangal Root Oil (Alpinia Spp.), Galbanum Oil (Ferula Spp.), Galbanum Resin (Ferula Spp.), Garlic Oil (Allium Sativum L.), Genet Absolute (Spartium Junceum L.), Genet Extract (Spartium Junceum L.), Gentian Root Extract (Gentiana Lutea L.), Geraniol, Elemi Gum (Canarium Spp.), Elemi Oil (Canarium Spp.), Erigeron Oil (Erigeron Candensis L.), Erythrobic Acid, Estragole, Estragon Oil (Artemisia Dracunculus L.), P-Ethoxybenzaldehyde, Ethyl Acetate, Ethyl Acetoacetate, Ethyl 2-Acetyl-3-Phenylpropionate, Ethyl Aconitate (Mixed Esters), Ethyl Acrylate, Ethyl Alcohol, Ethyl P-Anisate, Ethyl Anthranilate, Ethyl Benzoate, Ethyl Benzoylacetate, Alpha-Ethylbenzyl Butyrate, 2-Ethylbutyl Acetate, 2-Ethylbutyraldehyde, Ethyl Butyrate, Ethyl Isobutyrate, 2-Ethylbutyric Acid, Ethyl Cinnamate, Ethyl Cyclohexanepropionate, Ethyl Decanoate, Ethyl Formate, Ethyl 3(2-Furyl)Propanoate, 4-Ethylguaiacol, Ethyl Heptanoate, 2-Ethyl-2-Heptenal, Ethyl Hexanoate, Ethyl Lactate, Ethyl Laurate, Ethyl Levulinate, Ethyl 2-Methylbutyrate, Ethyl Methylphenylglycidate, Ethyl Myristate, Ethyl Nitrite, Ethyl Nonanoate, Ethyl 2-Nonynoate, Ethyl Octanoate, Ethyl Oleate, Ethyl Palmitate, Ethyl Phenylacetate, Ethyl 4-Phenylbutyrate, Ethyl 3-Phenylglycidate, Ethyl 3-Phenylpropionate, Ethyl Propionate, Ethyl Pyruvate, Ethyl Salicylate, Ethyl Sorbate, Ethyl Tiglate, Ethyl 10-Undecenoate, Ethyl Valerate, Ethyl Isovalerate, Ethyl Vanillin, Eucalyptol, Eucalyptus Oil (Eucalyptus Globulus Labille), Eugenol, Isoeugenol, Eugenyl Acetate, Isoeugenyl Acetate, Eugenyl Benzoate, Isoeugenyl Ethyl Ether, Eugenyl Formate, Isoeugenyl Formate, Eugenyl Methyl Ether, Isoeugenyl Methyl Ether, Isoeugenyl Phenylacetate, Farnesol, D-Fenchone, Fenchyl Alcohol, Fennel, Common (Foeniculum Vulgare Mill), Fennel, Sweet (Foeniculum Vulgare Mill, Var Dulce (D.C.), Fennel Oil, Sweet (Foeniculum Vulgare Mill. Var, Dulce Dc.), Fenugreek (Trigonella. Foenum-Graecum L.), Fenugreek Extract (Trigonella Foenum-Graecum L.), Fenugreek Oleoresin (Trigonella Foenumgraecum L.), Formic Acid, Fumaric Acid, Furfural, Furfuryl Acetate, Furfuryl Alcohol, 2-Furfurylidene Butyraldehyde, Furfuryl Mercaptan, 3-(2-Furyl)Acrolein, 4-(2-Furyl)-3-Buten-2-One, (2-Furyl)-2-Propanone, Fusel Oil, Refined, Galangal Root (Alpinia Spp.), Galangal Root Extract (Alpinia Spp.), Galangal Root Oil (Alpinia Spp.), Galbanum. Oil (Ferula Spp.), Galbanum Resin (Ferula Spp.), Garlic Oil (Allium Sativum L.), Genet Absolute (Spartium Junceum L.), Genet Extract (Spartium Junceum L.), Gentian Root Extract (Gentiana Lutea L.), Geraniol, Labdanum Absolute (Cistus Spp.), Labdanum Oil (Cistus Spp.), Labdanum Oleoresin (Cistus Spp.), Lactic Acid, Laurel Berries (Laurus Nobilis L.), Laurel Leaves Extract (Laurus Nobilis L.) Lauric Acid, Lauric Aldehyde, Lauryl Acetate, Lauryl Alcohol, Lavandin Oil (Lavandula Hybrida), Lavender (Lavandula Officinalis Chaix), Lavender Absolute (Lavandula Officinalis Chaix), Lavender Concrete (Lavandula Officinalis Chaix), Lavender Oil (Lavandula Officinalis Chaix), Lemon Extract (Citrus Limon (L.) Burm. F.), Lemongrass Oil, Lemon Oil, Lemon Oil Terpeneless (Citrus Limon (L.) Burm. F.), Levulinic Acid, Licorice Extract (Glycyrrhiza Spp.), Licorice Extract Powder (Glycyrrhiza Glabra L.), Licorice Root (Glycyrrhiza Glabra L.), Lime Oil, Lime Oil, Terpeneless (Citrus Aurantifolia (Christman) Swingle), D-Limonene, Linaloe Wood Oil (Bursera Delpechiana Poiss. And Other Bursera Spp.), Linalool, Linalyl Acetate, Linalyl Anthranilate, Linalyl Benzoate, Linalyl Butyrate, Linalyl Isobutyrate, Linalyl Cinnamate, Linalyl Formate, Linalyl Hexanoate, Linalyl Octanoate, Linalyl Propionate, Linalyl Isovalerate, Linden Flowers (Tilia Glabra Vent.), Locust Gum (Ceratonia Siliqua L.), Lovage (Levisticum Officinale Koch), Lovage Extract (Levisticum Officinale Koch), Lovage Oil (Levisticum Officinale Koch), Mace (Myristica Fragrans Houtt.), Mace Oil (Myristica Fragrans Houtt.), Mace Oleoresin (Myristica Fragrans Houtt.), L-Matic Acid, Maltol, Mandarin Oil, Expressed, Marigold, Pot (Calendula Officinalis L.), Marjoram Oleoresin (Majorana Hortensis Moench-Origanum Majorana L.), Marjoram, Pot (Origanum Vulgare L.), Marjoram Seed (Majorana Hortensis Moench-Origanum Majorana. L.), Marjoram, Sweet (Majorana Hortensis Moench-Origanum Majorana L.), Marjoram Oil, Sweet (Origanum Majorana), P-Mentha-1,8-Dien-7-O1, Menthol Racemic, (+)-Neoisomenthol, Menthone, Menthyl Acetate (isomer Unspecified), Menthyl Isovalerate, P-Methoxybenzaldehyde, 2-Methoxy-4-Methylphenol, 4-(P-Methoxyphenyl)-2-Butanone, 1-(P-Methoxyphenyl)-1-Penten-3-One, 1-(P-Methoxyphenyl)-2-Propanone, 2-Methoxy-4-Vinylphenol, Methyl Acetate, 4′-Methylacetophenone, 2-Methylallyl Butyrate, Methyl Anisate, O-Methylanisole, P-Methylanisole, Methyl Anthranilate, Methyl Benzoate, Alpha-Methylbenzyl Acetate, Alpha-Methylbenzyl Alcohol, Alpha-Methylbenzyl Butyrate, Alpha-Methylbenzyl Isobutyrate, Alpha-Methylbenzyl Formate, Alpha-Methylbenzyl Propionate, Methyl P-Tert-Butylphenylacetate, 2-Methylbutyraldehyde, 3-Methylbutyraldehyde, Methyl Butyrate, Methyl Isobutyrate, 2-Methylbutyric Acid, Methyl Cellulose, Alpha-Methylcinnamaldehyde, Methyl Cinnamate, 6-Methylcoumarin, Methylcyclopentenolone, 4-(3,4-Methylenedioxyphenyl)-2-Butanone, 5-Methylfurfural, Methyl 2-Furoate, 2-Methyl-3(2-Furyl)Acrolein, Methyl Heptanoate, 2-Methylheptanoic Acid, 6-Methyl-5-Hepten-2-One, Methyl Hexanoate, Methyl 2-Hexenoate, Methyl P-Hydroxybenzoate, Methyl-Alpha-Ionone, Methyl-Beta-Ionone, Methyl-Delta-Ionone, Alpha-Iso-Methylionone, Methyl Laurate, Methyl Mercaptan, Methyl O-Methoxybenzoate, Methyl N-Methylanthranilate, Methyl 2-Methylbutyrate, Methyl 3-Methylthiopropionate, Methyl 4-Methylvalerate, Methyl Myristate, Methyl Beta-Naphthyl Ketone, Methyl Nonanoate, Methyl 2-Nonenoate, Methyl 2-Nonynoate, 2-Methyloctanal, Methyl Octanoate, Methyl 2-Octynoate, 4-Methyl-2,3-Pentanedione, 4-Methyl-2-Pentanone, Beta-Methylphenethyl Alcohol, Methyl Phenylacetate, 3-Methyl-4-Phenyl-3-Butene-2-One, 2-Methyl-4-Phenyl-2-Butyl Acetate, 2-Methyl-4-Phenyl-2-Butyl Isobutyrate, 2-Methyl-4-Phenylbutyraldehyde, 3-Methyl-2-Phenylbutyraldehyde, Methyl 4-Phenylbutyrate, 4-Methyl-1-Phenyl-2-Pentanone, Methyl 3-Phenylpropionate, Methyl Propionate, 2-Methyl-3-(P-Isopropylphenyl)Propionaldehyde, 6-Methylquinoline, Methyl Salicylate, Methyl Sulfide, 3-(Methylthio)Propionaldehyde, 2-Methyl-3-Tolyipropionaldehyde, 2-Methylundecanal , Methyl 9-Undecenoate, Methyl 2-Undecynoate, Methyl Valerate, Methyl Isovalerate, 2-Methylvaleric Acid, Mimosa Absolute (Acacia Decurrens Willd. Var. Dealbata), Monosodium Glutamate, Mountain Maple Extract Solid (Acer Spicatum Lam.), Musk Tonquin (Moschus Moschiferus L.), Mustard, Brown (Brassica Spp.), Mustard, Yellow (Brassica Spp.), Myrcene, Myristaldehyde, Myristic Acid, Myrrh Gum (Commiphora Spp.), Myrrh Oil (Commiphora Spp.), Beta-Naphthyl Anthranitate, Beta-Naphthyl Ethyl Ether, Naringen Extract (Citrus Paradisi Macf.), Nerol, Neroli Bigarde Oil (Citrus Aurantium L.), Nerolidol (Isomer Unspecified), Neryl Acetate, Neryl Butyrate, Neryl Isobutyrate, Neryl Formate, Neryl Propionate, Neryl Isovalerate, Nitrous Oxide, 2,6-Nonadien-1-O1, Gamma-Nonalactone, Nonanal, 1,3-Nonanediol Acetate (Mixed Esters), Nonanoic Acid, 2-Nonanone, Nonanoyl 4-Hydroxy-3-Methoxybenzylamide, Nonyl Acetate, Nonyl Alcohol, Nonyl Octanoate, Isovalerate, Nutmeg (Myristica Fragrans Houtt.), Nutmeg Oil, Oak Chips Extract (Quercus Alba L.), Oakmoss Absolute (Evernia Spp.), Gamma-Octalactone, Octanal, Octanal Dimethyl Acetal, Octanoic Acid, 1-Octanol, 2-Octanol, 2-Octanone, 3-Octanone, 3-(Hydroxymethyl)-2-Heptanone, 1-Octen-3-O1, Octyl Acetate, Octyl Butyrate, Octyl Isobutyrate, Octyl Formate, Octyl Heptanoate, Octyl Octanoate, Octyl Phenylacetate, Octyl Propionate, Octyl Isovalerate, Oleic Acid, Olibanum Oil (Boswellia Spp.), Onion Oil (Allium Cepa L.), Onion Flakes, Onion Powder, onion Extract, Orange Blossoms Absolute, Orange Flowers (Citrus Aurantium L.), Orange Leaf Absolute (Citrus Aurantium L.), Orange Oil Distilled (Citrus Sinensis (L.) Osbeck), Orange Oil Terpeneless (Citrus Sinensis (L.) Osbeck), orange Peel Oil, Bitter (Citrus Aurantium L.), Orange Peel, Sweet, Extract (Citrus Sinensis L. Osbeck), Orange Peel Oil, Sweet (Citrus Sinensis (L.) Osbeck), Orange Peel, Sweet, Oil, Terpeneless (Citrus Sinensis L. Osbeck), Oregano (Lippia Spp.), Origanum Oil (Extractive) (Thymus Capitatus L. Hoffmanns & Link), Orris Concrete Liquid Oil (Iris Florentina L.), Orris Root Extract (Iris Florentina L.), Palmarosa. Oil (Cymbopogon Martini (Roxb.) Stapt), Palmitic Acid, Paprika (Capsicum Annuum L.), Paprika Oleoresin (Capsicum Annuum L.), Parsley (Petroselinum Crispum (Miller) Nyman-P. Sativum Hoffm.), Parsley Oil, Parsley Oleoresin (Petroselinum Spp.), Parsnip (Pastinaca Sativa), Parsnip, Dehydrated, Parsnip Extract, Patchouly Oil, Pennyroyal Oil (Mentha Pulegium L.), Omega-Pentadecalactone, 2,3-Pentanedione, 2-Pentanone, 4-Pentenoic Acid, Pepper, Black (Piper Nigrum L.), Pepper, Black, Oil (Piper Nigrum L.), Pepper, Black, Oleoresin (Piper Nigrum L.), Pepperment Leaves (Mentha Piperita L.), Peppermint Oil, Pepper (Capsicum Frutescens L. (Capsicum Annuum L.)), Pepper, Red (Capsicum Frutescens L. (Capsicum Annuum L.)), Pepper, Green (Capsicum Frutescens L. (Capsicum Annuum L.)), Pepper, Orange (Capsicum Frutescens L. (Capsicum Annuum L.)), Pepper, Yellow (Capsicum Frutescens L. (Capsicum Annuum L.)), Pepper, White (Piper Nigrum L.), Pepper, White, Oil (Piper Nigrum L.), Pepper, White, Oleoresin (Piper Nigrum L.) Petitgrain, Lemon, Oil (Citrus Limon L Burm. F), Petitgrain Mandarin Oil (Citrus Reticulata Blanco Var. Mandarin), Petitgrain Paraguay Oil, Alpha-Phellandrene, Phenethyl Acetate, Phenethyl Alcohol, Phenylethyl Anthranilate, Phenethyl Benzoate, Phenethyl Butyrate, Phenethyl Isobutyrate, Phenethyl Cinnamate, Phenethyl Formate, Phenethyl 2-Furoate, Phenethyl Phenylacetate, Phenethyl Propionate, Phenethyl Salicylate, Phenethyl Senecioate, Phenethyl Tiglate, Phenethyl Isovalerate, Phenoxyacetic Acid, 2-Phenoxyethyl Isobutyrate, Phenylacetaldehyde, Phenylacetaldehyde 2,3-Butylene Glycol Acetal, Phenylacetaldehyde Dimethyl Acetal, Phenylacetaldehyde Glyceryl Acetal, Phenylacetic Acid, 4-Phenyl-2-Butanol, 4-Phenyl-3-Buten-2-O1, 4-Phenyl-3-Buten-2-One, 4-Phenyl-2-Butyl Acetate, 1-Phenyl-3-Methyl-3-Pentanol, 1-Phenyl-1-Propanol, 3-Phenyl-1-Propanol, 2-Phenylpropionaldehyde, 3-Phenylpropionaldehyde, 2-Phenylpropionaldehyde Dimethyl Acetal, 3-Phenylpropionic Acid, 3-Phenylpropyl Acetate, 2-Phenylpropyl Butyrate, 2-Phenylpropyl Isobutyrate, 3-Phenylpropyl Isobutyrate, 3-Phenylpropyl Cinnamate, 3-Phenylpropyl Formate, 3-Phenylpropyl Hexanoate, 3-Phenylpropyl Propionate, 2-(3-Phenylpropyl)Tetrahydrofuran, 3-Phenylpropyl Isovalerate, Phosphoric Acid, Pimenta Leaf Oil, Alpha-Pinene, Beta-Pinene, Pine Needle, Dwarf, Oil (Pinus Mugo Turra Var. Pumilio (Haenk0 Zenari), Pine Needle Oil (Abies Spp.), Pine Scotch Oil (Pinus Sylvestris L.), Pine Tar Oil (Pinus Palustris Mill. And Other Pinus Spp.), Piperidine, Piperine, D-Piperitone, Piperonyl, Piperonyl Acetate, Piperonyl Isobutyrate, Pipsissewa Leaves Extract (Chimaphila Umbellata Nutt.), Polysorbate 20, Polysorbate 60, Polysorbate 80, Pomegranate Bark Extract (Punica Granatum L.), Poppy Seed (Papaver Somniferum L.), Potassium Acetate, Potassium Sorbate, Propenylguaethol, Propionaldehyde, Propionic Acid, Propyl Acetate, Isopropyl Acetate, P-Isopropylacetophenone, Propyl Alcohol, Isopropyl Alcohol, P-Propylanisole, Propyl Benzoate, Isopropyl Benzoate, P-Isopropylbenzyl Alcohol, Propyl Butyrate, Isopropyl Butyrate, Propyl Isobutyrate, Isopropyl Isobutyrate, Propyl Cinnamate, Isopropyl Cinnamate, Propylene Glycol, Propylene Glycol Alginate, Propylene Glycol Stearate, Propyl Formate, Isopropyl Formate, Propyl 2-Furanacrylate, Propyl 2-Furoate, Propyl Gallate, Propyl Heptanoate, Propyl Hexanoate, Isopropyl Hexanoate, Propyl P-Hydroxybenzoate, 3-Propylidenephthalide, Alpha-Propylphenethyl Alcohol, P-Isopropyl Phenylacetaldehyde, Propyl Phenylacetate, Isopropyl Phenylacetate, 3-(P-Isopropylphenyl)Propionaldehyde, Propyl Propionate, Isopropyl Propionate, Propyl Isovalerate, Isopropyl Isovalerate, Isopulegol, Pulegone, Isopulegone, Isopulegyl Acetate, Pyridine, Pyroligneous Acid, Pyroligneous Acid, Extract, Pyruvaldehyde, Pyruvic Acid, Quassia Extract (Picrasma Excelsa (Sw.) Planch.-Quassia Amara. L.), Quebracho Bark Extract, Quillala (Quillaja Saponaria Molina), Quince Seed Extract (Cydonia Spp.), Quinine Bisulfate, Quinine Hydrochloride, Quinine Sulfate, Isoquinoline, Rhatany Extract (Krameria Spp.), Rhodinol, Rhodinyl Acetate, Rhodinyl Butyrate, Rhodinyl Isobutyrate, Rhodinyl Formate, Rhodinyl Phenylacetate, Rhodinyl Propionate, Rhodinyl Isovalerate, Rose Absolute (Rosa Spp.), Rose Oil (Rosa Damascena Mill.), Rose Hips Extract (Rosa Spp.), Rosemary (Rosemarinus Officinalis L.), Rosemary Oil (Rosemarinus Officinalis L.), Rose Water, Stronger (Rosa Centifolia L.), Rue (Ruta Graveolens L.), Rue Oil (Ruta Graveolens L.), Rum Ether, Saccharine, Sodium Salt, Saffron (Crocus Sativus L.), Saffron Extract (Crocus Sativus L.), Sage (Salvia Officinalis L.), Sage Oil (Salvia Officinalis L.), Sage Oleoresin (Salvia Officinalis L.), Sage Oil, Spanish (Salvia Lavandulaefolia Vahl.), Saticylaldehyde, Sandalwood Yellow Oil (Santalum Album L.), Santalol (Alpha And Beta), Santalyl Acetate, Santalyl Phenylacetate, Sarsaparilla Extract (Smilax Spp.), Sassafras Bark Extract (Safrol-Free) (Sassafras Albidum (Nutt.) Nees), Sassafras Leaves (Safrol-Free) (Sassafras Albidum (Nutt.) Nees), Savory, Summer (Satureja Hortensis L.), Savory Summer Oil (Satureja Hortensis L.), Savory, Summer, Oleoresin (Satureja Hortensis L.), Savory, Winter (Satureja Montana L.), Savory Winter Oil (Satureja Montana L.), Savory, Winter, Oleoresin (Satureja Montana L.), Schinus Molle Oil (Schinus Molle L.), Skatole, Sloe Berries (Prunus Spinosa L.), Sloe Berries Extract (Prunus Spinosa L), Sloe Berries Extract Solid (Prunus Spinosa L.), Snakeroot Oil, Canadian (Asarum Canadense L.), Sodium Acetate, Sodium Benzoate, Sodium Citrate, Sodium Hexametaphosphate, Sorbitan Monostearate, D-Sorbitol, Sour Cream, Sour Cream Solids, Spearmint (Mentha Spicata L.), Spearmint Extract (Mentha Spicata L.), Spearmint Oil (Mentha Spicata L.), Spike Lavender Oil (Lavandula Spp.), Spruce Oil (Tsuga And Picea Spp.), Stearic Acid, Storax (Liquidambar Spp.), Styrax Extract (Liquidambar Spp.), Sucrose Octaacetate, Sulfur Dioxide, Tagetes Oil (Tagetes Erecta L.; T. Patula L.; Or T. Glandulifera Schrank), Tangerine Oil (Citrus Reticulata Blanco), Tannic Acid (Quercus Spp.), Tarragon (Artemisia Dracunculus L.), Tartaric Acid (D-, L-, D1-Meso-), Alpha-Terpineol, Terpinolene, Terpinyl Acetate (Isomer Mixture), Beta-Terpinyl Anthranilate, Terpinyl Butyrate, Terpinyl Isobutyrate, Terpinyl Cinnamate, Terpinyl Formate, Terpinyl Propionate, Terpinyl Isovalerate, Tetrahydrofurfuryl Acetate, Tetrahydrofurfuryl Alcohol, Tetrahydrofurfuryl Butyrate, Tetrahydrofurfuryl Propionate, 3,4,5,6-Tetrahydropseudoionone, Tetrahydrolinalool, Tetramethyl Ethylcyclohexenone (Mixture Of Isomers), 2-Thienyl Mercaptan, Thyme (Thymus Vulgaris L.), Thyme Oil (Thymus Vulgaris L.), Thyme, White, Oil (Thymus Vulgaris L.), Thymol, Tolualdehyde Glyceryl Acetal (Mixed O-, M-, P-), Tolualdehydes (Mixed O,M,P), Tolu, Balsam, Extract (Myroxylon Spp.), Tolu, Balsam, Gum (Myroxylon Spp.), P-Tolylacetaldehyde, O-Tolyl Acetate, P-Tolyl Acetate, 4-(P-Tolyl)-2-Butanone, P-Tolyl Isobutyrate, P-Tolyl Laurate, P-Tolyl Phenylacetate, P-Tolyl Phenylacetate, 2-(P-Tolyl)Propionaldehyde, Tragacanth Gum (Astragalus Spp.), Tributyl Acetylcitrate, Tricalcium Phosphate, 2-Tridecenal, Triethyl Citrate, Tuberose Oil (Polianthes Tuberosa L.), Turmeric (Curcuma Longa L.), Turmeric Extract (Curcuma Longa L.), Turmeric Oleoresin (Curcuma Longa. L.), Turpentine Gum (Pinus Spp.), Turpentine, Steam Distilled (Pinus Spp.), 2,3-Undecadione, Gamna-Undecalactone, Undecanal, 2-Undecanone, 9-Undecenal, 10-Undecenal, 10-Undecen-1-Y1 Acetate, Undecyl Alcohol, Valeraldehyde, Valerian Root Extract (Valeriana Officinalis L.), Valerian Root Oil (Valeriana Officinalis Valerie Acid, isovaleric Acid, Gamma-Valerolactone, Vanilla (Vanilla Spp.), Vanilla Extract (Vanilla Spp.), Vanilla Oleoresin (Vanilla Spp.), Vanillin, Vanillin Acetate, Veratraldehyde, Violet Leaves Absolute (Viola Odorata L.), Walnut Hull Extract (Juglans Spp.), Wintergreen Extract (Gaultheria Procumbens L.), Wintergreen Oil (Gaultheria Procumbens L.), Wormwood (Artemisia Absinthium L.), Wormwood Extract (Artemisia Absinthium L.), Wormwood Oil (Artemisia Absinthium L.) Yarrow Herb (Achillea Millefolium L. ), Yerba Santa Fluid Extract (Eriodictyon Californicum (Hook And Am) Torr, Ylang Ylang Oil (Cananga Odorata Hook. F. And Thomas), Yucca Joshua Tree (Yucca Brevifolia Engelm.), Yucca Mohave Extract (Yucca Spp.), Zedoary (Curcuma Zedoaria (Berg.) Rosc.), Zedoary Bark Extract (Curcuma Zedoaria (Berg.) Rosc.), Zingerone, Acetaldehyde Butyl Phenethyl Acetal, Acetylpyrazine, Allyl Methyl Disulfide, 2-Benzofurancarboxaldehyde, Biphenyl, Butylamine, Sec-Butyl Ethyl Ether, 2-Isobutyl-3-Methoxypyrazine, 2-Isobutyl-3-Methylpyrazine, 2-Isobutylthiazole, 2-Trans,4-Trans-Decadienal, 2,3-Diethylpyrazine, 2,6-Dimethoxyphenol, 3,4-Dimethoxy-1-Vinylbenzene, P-Alpha-Dimethylbenzyl Alcohol, 2,6-Dimethyl-4-Heptanol, 2,6-Dimethyl-10-Methylene-2,6,11-Dodecatrienal, 3,7-Dimethyl-6-Octenoic Acid, 2,4-Dimethyl-2-Pentenoic Acid, P,Alpha-Dimethylstyrene, 2,4-Dimethyl-5-Vinylthiazole, 2,2′-(Dithiodimethylene)-Difuran, 1-Ethyl-2-Acetylpyrrole, Ethyl Trans-2,Cis-4-Decadienoate, 2-Ethyl-3,(5 Or 6)-Dimethylpyrazine, 3-Ethyl-2,6-Dimethylpyrazine, 2-Ethyl-1-Hexanol, 3-Ethyl-2-Hydroxy-2-Cyclopenten-1-One, 5-Ethyl-3-Hydroxy-4-Methyl-2(5h)-Furanone, 2-Ethyl-5-Methylpyrazine, 2-Ethyl-3-Methylpyrazine, P-Ethylphenol, Ethyl (P-Tolyloxy)Acetate, 2-Furanmethanethiol Formate, Furfuryl Methyl Ether, Furfuryl Methyl Sulfide, Furfuryl Isopropyl Sulfide, Furfuryl Thioacetate, 2-Furyl Methyl Ketone, (2e,4e)-Heptadienal, Trans-2-Heptenal, Nootkatone, Delta-Hexalactone, 3,4-Hexanedione, Trans-2-Hexenoic Acid, 3-Hexenoic Acid, Cis-3-Hexen-1-Y1 Acetate, Hexyl Isobutyrate, 1-Hydroxy-2-Butanone, 4-Hydroxy-2,5-Dimethyl-3(2h)-Furanone, Gamma-Ionone, P-Menthan-2-One, P-Mentha-8-Thiol-3-One, P-Menth-1-Ene-9-A1, P-Menth-1-En-3-O1, 2-Mercaptopropionic Acid, O-Methoxycinnamaldehyde, P-Methoxy-Alpha-Methylcinnamaldehyde, 2,5 Or 6-Methoxy-3-Methylpyrazine (Mixture Of Isomers), 1-Methyl-2-Acetylpyrrole, Methylated Silica, 4-Methylbiphenyl, 3-Methylcrotonic Acid, 2-Methyl-3-Furanthiol, 2-Methyl-3-,5 Or 6-(Furfurylthio)Pyrazine (Mixture Of Isomers), 5-Methyl-2,3-Hexanedione, 2-Methylhexanoic Acid, 2-Methyl-5-Methoxythiazole, 1-Methylnaphthalene, 2-Methyl-2-Pentenal, 2-Methyl-2-Pentenoic Acid, 3-Methyl-2-(2-Pentenyl)-2-Cyclopenten-1-One, Alpha-Methylphenethyl Butyrate, Methyl Phenethyl Ether, 5-Methyl-2-Phenyl-2-Hexenal, 4-Methyl-2-Phenyl-2-Pentenal, Methyl Propyl Disulfide, Methyl 2-Pyrrolyl Ketone, 5-Methylquinoxaline, 4-Methyl-5-Thiazoleethanol, 4-Methyl-5-Thiazoleethanol Acetate, 2-Methylthioacetaldehyde, 1-(Methylthio)-2-Butanone, (Methylthio)Methylpyrazine (Mixture Of Isomers), 5-Methyl-2-Thiophenecarboxaldehyde, O-(Methylthio)-Phenol, 2-Methyl-5-Vinylpyrazine (Re-Gras), 2,4-Nonadienal, 2-Nonenal, Delta-Octalactone, 2-Octenal, Paraffin Wax, 2,4-Pentadienal, 2-Pentenal, Isopentylamine, Phenethylamine, Phenethyl Hexanoate, Phenethyl Octanoate, Phenol, 2-Phenyl-2-Butenal, Phenyl Disulfide, 1-Phenyl-1,2-Propanedione, Propenyl Propyl Disulfide, Propyl Disulfide, Isopropyl Tiglate, Pyrazine Ethanethiol, Pyrazinyl Methyl Sulfide, 2-Pyridinemethanethiol, 4,5,6,7-Tetrahydro-3,6-Dimethylbenzofuran, Tetrahydro-4-Methyl-2-(2-Methylpropen-1-Y1)Pyran, 2,3,5,6-Tetramethylpyrazine, 2,2′-(Thiodimethylene)-Difuran, 4-Thujanol, O-Toluenethiol, Trimethylamine, P-Alpha,Alpha-Trimethylbenzyl Alcohol, 1-(2,6,6-Trimethyl-1-Cyclohexen-1-Y1)-2-Buten-1-One, 2,3,5-Trimethylpyrazine, Undecanoic Acid, 2-Undecanol, 10-Undecenoic Acid, 2,6-Xylenol, 2-Acetyl-3-Ethylpyrazine, 2-Acetylpyridine, Beta-Alanine, Allyl Methyl Trisulfide, Arabinogalactan, L-Arabinose, Benzothiazole, Bis(2-Methyl-3-Furyl) Disulfide, Bis(2-Methyl-3-Furyl) Tetrasulfide, 2-Sec-Butylcyclohexanone, Cyclopentanethiol, L-Cysteine, 4-Decenal, Diallyl Trisulfide, 4,5-Dihydro-3(2h)Thiophenone, 2,4-Dimethyl-5-Acetylthiazole, 3,4-Dimethyl-1,2-Cyclopentadione, 3,5-Dimethyl-1,2-Cyclopentadione, Spiro(2,4-Dithia-1-Methyl-8-Oxabicyclo(3.3.0)Octane-3,3′-(1′-Oxa-2′-Methyl)-Cyclopentane), 2,3-Dimethylpyrazine, 2,5-Dimethylpyrazine, 2,6-Dimethylpyrazine, 4,5-Dimethylthiazole, Dimethyl Trisulfide, Dipropyl Trisulfide, Disodium Succinate, Ethyl 2,4-Dioxohexanoate, Ethyl 2-Mercaptopropionate, 2-Ethyl(Or Methyl)-(3,5 And 6)-Methoxypyrazine, 2-Ethylpyrazine, Ethyl Thioacetate, Furfuryl 3-Methylbutanoate, N-Furfurylpyrrrole, L-Glutamic Acid, Glyceryl Tripropanoate, Glycine, 2-Heptanol, 4-Heptenal, 3-Hexanone, 4-Hydroxybutanoic Acid Lactone, 3-(Hydroxymethyl)-2-Octanone, 4-Hydroxy-3-Pentenoic Acid Lactone, 5Hydroxyundecanoic Acid Lactone, D,L-Isoleucine, Isopropenylpyrazine, L-Leucine, 3-Mercapto-2-Butanone, 2-Mercaptomethylpyrazine, 3-Mercapto-2-Pentanone, D,L-Methionine, Methoxypyrazine, 2-Methyl-1-Butanethiol, 3-Methyl-2-Butanethiol, 1-Methyl-2,3-Cyclohexadione, 5h-5-Methyl-6,7-Dihydrocyclopenta(B)Pyrazine, 3-(5-Methyl-2-Furyl)-Butanal, Methyl Propyl Trisulfide, 2-Methylpyrazine, Methyl Thiobutyrate, Methyl 2-Thiofuroate, 3-Methylthiopropyl Isothiocyanate, 4-Methyl-5-Vinylthiazole, 2-Naphthalenthiol, 2-Nonanol, 2-Pentanol, 2-Pentylfuran, 3-Phenyl-4-Pentenal, L-Proline, Tetrahydrofurfuryl Cinnamate, 5,6,7,8-Tetrahydroquinoxaline, Thiamine Hydrochloride, 2-Thienyl Disulfide, 3,5,5-Trimethyl-1-Hexanol, 2,4,5-Trimethylthiazote, Acetone, 2-Acetyl-3,5 (And 6)-Dimethylpyrazine, 2-Acetylthiazole, Allyl Thiopropionate, Benzyl Trans-2-Methyl-2-Butenoate, Bisabolene, Butan-3-One-2-Y1 Butanoate, 3-Butylidenephthalide, Di(Butan-3-One-1-Y1) Sulfide, 2,3-Diethyl-5-Methylpyrazine, Difurfuryl Ether, 5,7-Dihydro-2-Methylthieno(3,4-D)Pyrimidine, 3,7-Dimethylocta-2,6-Dienyl 2-Ethylbutanoate, 2-Ethoxythiazole, Ethyl 2-Ethyl-3-Phenylpropanoate, Ethyl 3-Hexenoate, Ethyl 3-Methylthiopropionate, Ethyl Cis-4-Octenoate, 2-Ethylthiophenol, Furfuryl Propionate, Furfuryl Thiopropionate, Heptanoic Acid, 4-Heptenal Diethyl Acetal, 3-Heptyldihydro-5-Methyl-2(3h)-Furanone, 3-Hexanol, 4-Hexene-3-One, Cis-3-Hexenyl Formate, N-Hexyl 2-Butenoate, 6-Hydroxy-3,7-Dimethyloctanoic Acid Lactone, Hydroxynonanoic Acid, Delta-Lactone, 2-Keto-4-Butanethiol, 2-Methoxy-3(5 And 6)-Isopropylpyrazine, 2-Methylbutyl 2-Methylbutyrate, 3-Methyl-2-Cyclohexen-1-One, Methyl 3,7-Dimethyl-6-Octenoate, Methyl Furfuryl Disulfide, 6-Methyl-3,5-Heptadien-2-One, Methyl 3-Hexenoate, 5-Methyl-5-Hexen-2-One, 2-Methyl-5-(Methylthio)Furan, Methyl Cis-4-Octenoate, 4-Methyl-3-Penten-2-One, 2-Methylpropyl 3-Methylbutyrate, 2-(2-Methylpropyl)Pyridine, 3-(2-Methylpropyl)Pyridine, 2-(1-Methylpropyl)Thiazole, 2-Methyltetrahydrofuran-3-One, 3-(Methylthio)Butanal, 4-(Methylthio)-2-Butanone, 4-(Methylthio)-4-Methyl-2-P entanone, Nona-2-Trans-6-Cis-Dienal, 2,6-Nonadienal Diethyl Acetal, Trans-2-Nonen-1-O1, 9,12-Octadecadienoic Acid (48%) And 9,12,15-Octadecatrienoic Acid (52%), 3-Oxobutanal Dimethyl. Acetal, 1-Penten-3-One, 2-Pentylpyridine, Phenylacetaldehyde Diisobutyl Acetal, Propyl Thioacetate, Pyrrole, P-Tolyl 3-Methylbutyrate, 2-Tridecanone, 2,6,6-Trimethylcyclohexa-1,3-Dienyl Methanal, 1,3,3-Trimethyl-2-Norbornanyl Acetate, 3-Acetyl-2,5-Dimethylfuran, 2-Butyl-2-Butenal, N-Butyl 2-Methylbutyrate, 3-Ethylpyridine, 2-Formyl-6,6-Dimethylbicyclo(3.1.1)Hept-2-Ene, Alpha-Furfuryl Octanoate, Alpha-Furfuryl Pentanoate, Glyceryl Tribenzoate, 2-Hepten-4-One, 3-Hepten-2-One, 2-Heptylfuran, Cis-3-Hexenyl Butyrate, Cis-3-Hexenyl Hexanoate, Capsaicin, 2-Hydroxymethyl-6,6-Dimethylbicyclo(3.1.1)Hept-2-Enyl Formate, 2-Isopropyl-5-Methyl-2-Hexenal, 2-Methyl-2-Butenal, Methyl Dihydrojasmonate, 5-Methyl-3-Hexen-2-One, Methyl Jasmonate, Methyl Linoleate (48%) Methyl Linolenate (52%) Mixture, Methyl 4-(Methylthio)Butyrate, 2-Methylpentanal, 4-(Methylthio)Butanal, 3-(Methylthio)Propanol, 3-Octen-2-One, 3-Penten-2-One, Pentyl 2-Furyl Ketone, Propylene Glycol Dibenzoate, 1-(2,6,6-Trimethylcyclohexa-1,3-Dienyl)-2-Buten-1-One, 2,6,6-Trimethylcyclohex-2-Ene-1,4-Dione, 2,4-Undecadienal, 2-Undecenal, 3-Acetylpyridine, Cycloheptadeca-9-En-1-One, 1,1-Dimethoxyethane, 2,4-Dimethylbenzaldehyde, Ethyl 3-Hydroxybutyrate, Trans,Trans-2,4-Hexadienal, 4-Hexen-1-O1, Isobutyl 2-Butenoate, 2-Methoxy-3-(1-Methylpropyl)Pyrazine, 3-Methyl-1-Cyclopentadecanone, 1-Methyl-1-Cyclopenten-3-One, 1-Methyl-3-Methoxy-4-Isopropylbenzene, 3-Methylpentanoic Acid, 3-(Methylthio)-1-Hexanol, Myrtenol, 3-Nonanone, 2,2,4-Trimethyl-1,3-Oxacyclopentane, 2,6,10-Trimethyl-2,6,10-Pentadecatrien-14-One, Valencene, D,L-(3-Amino-3-Carboxypropyl)Dimethylsulfoniurn Chloride, Dehydrodihydroionol, Dehydrodihydroionone, Dicyclohexyl Disulfide, 1,4-Dimethyl-4-Acetyl-1-Cyclohexene, 2,5-D imethyl-2,5-Dihydroxy-1,4-D ithiane, 2,5-Dimethyl-3-Furanthiol, Dodecyl Isobutyrate, 3-Ethyl-2-Hydroxy-4-Methylcyclopent-2-En-1-One, 5-Ethyl-2-Hydroxy-3-Methylcyclopent-2-En-1-One, N-Ethyl-2-Isopropyl-5-Methylcyclohexane Carboxamide, Ethyl 2-Methyl-3-Pentenoate, Hexyl Phenylacetate, 2-Hydroxy-2-Cyclohexen-1-One, 2-Hydroxy-3,5,5-Trimethyl-2-Cyclohexenone, D,L-Isomenthone, 2-Isopropylphenol, Maltyl Isobutyrate, 4-Methylpentanoic Acid, 2-Methyl-3-Pentenoic Acid, Cis-6-Nonen-1-O1, 2-Trans-6-Trans-Octadienal, Cis-3-Octen-1-O1, 2-Phenyl-3-Carbethoxy Furan, Propiophenone, 1,5,5,9-Tetramethyl-13-Oxatricyclo(8.3.0.0(4,9))Tridecane, Thiogeraniol, 2,2,6-Trimethylcyclohexanone, 2,6,6-Trimethyl-1-Cyclohexen-1-Acetaldehyde, Trithioacetone, Bis(2,5-Dimethyl-3-Furyl) Disulfide, 2,3-Butanedithiol, 1-Butanethiol, Candefilla Wax (Wax From Stems And Branches Of Euphorbia Cerifera), O-Cresol, S-(2,5-Dimethyl-3-Furyl)Thio-2-Furoate, 2,5-Dimethyl-3-Thioisovalerylfuran, 2,8-Dithianon-4-En-4-Carboxaldehyde, 1,2-Ethanedithiol, O-(Ethoxymethyl)Phenol, Ethyl Trans-2-Butenoate, Ethyl Maltol, Ethyl 2-Methylpentanoate, Ethyl 2-Methyl-4-Pentenoate, Ethyl Octadecanoate, 2-Ethyl-1,3,3-Trimethyl-2-Norbornanol, Ethyl Undecanoate, Trans-3-Heptenyl Acetate, Trans-3-Heptenyl 2-Methylpropanoate, 1,6-Hexanedithiol, Cis-4-Hexenal, 3-Hexenyl 2-Methylbutanoate, 3-Hexenyl 3-Methylbutanoate, Hexyl 2-Methylbutanoate, Hexyl Isovalerate, Linalyl Phenylacetate, 2-Mercapto-3-Butanol, 2,3 Or 10-Mercaptopinane, Methyl Benzyl Disulfide, 3-Methytbutyl 2-Methylbutanoate, 2-Methylbutyl 3-Methylbutanoate, 3-Methylbutyl 2-Methylpropanoate, Methyl 3-Hydroxyhexanoate, Alpha-Methyl-Beta-Hydroxypropyl Alpha-Methyl-Beta-Mercaptopropyl Sulfide, 4-Methyl-2-Pentenal, 2-Methyl-4-Pentenoic Acid, 2-Methyltetrahydrothiophen-3-One, 1,9-Nonanedithiol, 1,8-Octanedithiol, 1-Octen-3-One, Trans-2-Octen-1-Y1 Acetate, Trans-2-Octen-1-Y1 Butanoate, Octyl 2-Furoate, 2-Phenyl-4-Pentenal, 1,2-Propanedithiol, Propanethiol, O-Propylphenol, Pyrrolidine, 3,5,5-Trimethylhexanal, 2,4,5-Trimethyl-Delta-3-Oxazoline, 2-Acetoxy-3-Butanone, 1,2-Butanedithiol, 1,3-Butanedithiol, M-Cresol, Cyclohexanecarboxylic Acid, 3-Decen-2-One, Diallyl Polysulfides, 1,2-Di((1′-Ethoxy)Ethoxy)Propane, 2,3-Dimethylbenzofuran, Dimethyl Disulfide, 2,6-Dimethyl-4-Heptanone, 2,6-Dimethyl-3-((2-Methyl-3-Furyl)Thio)-4-Heptanone, 3,7-Dimethyl-1,3,6-Octatriene, 2,6-Dimethylpyridine, 3,5-Dimethyl-1,2,4-Trithiolane, 6,10-Dimethyl-5,9-Undecadien-2-One, Ethylene Brassylate, Ethyl Cyclohexanecarboxylate, Ethyl 3-Hydroxyhexanoate, 5-Ethyl-2-Methylpyridine, 3-Heptanol, 2-Hydroxyacetophenone, 6-Hydroxydihydrotheaspirane, 3-Hydroxy-2-Pentanone, Isoamyl Acetoacetate, Isojasmone, Isophorone, 5-Isopropyl-2-Methylpyrazine, 2-Isopropyl-4-Methylthiazole, Isopropyl Myristate, P-Mentha-1,8-Dien-7-Al, P-Mentha-1,3-Diene, P-Mentha-1,4-Diene, P-Mentha-1,4(8)-Dien-3-One, P-Mentha-1,8-Dien-7-Y 1 Acetate, P-Menthan-2-O1, P-Menth-3-En-1-O1, P-Menth-8-En-1-O1, P-Menth-8-En-2-One, 1-P-Menthen-9-Y1 Acetate, P-Methoxycinnamaldehyde, Methyl Cyclohexanecarboxylate, 2-Methyl-3,5 Or 6-Ethoxypyrazine, 3((2-Methyl-3-FurylThio)-4-Heptanone, 4-((2-Methyl-3-Furyl)Thio)-5-Nonanone, 5-Methylhexanoic Acid, Methyl 2-Methyl-3-Furyl Disulfide, 4-Methylnonanoic Acid, 4-Methyloctanoic Acid, Methyl 1-Propenyl Disulfide, 3-Methyl-5-Propyl-2-Cyclohexen-1-One, 2-Methyl-4-Propyl-1,3-Oxathiane, 1,4-Nonanediol Diacetate, Cis-6-Nonenal, 3-Octanol, 1-Octen-3-Y1 Acetate, 3-Octyl Acetate, 1-Penten-3-O1, L-Phenylalanine, 2-Ph enyl-3-(2-Furyl)Prop-2-Enal, 2(10)-Pinen-3-O1, 1,3-Propanedithiol, Resorcinol, Delta-Tetradecalactone, Theobromine, (2,2,3-Trimethylcyclopent-3-En-1-Y1)Acetaldehyde, 1,2,3-Tris((1′-Ethoxy)Ethoxy)Propane, Verbenol, 2,5-Xylenol, 3,4-Xylenol, Benzyl Methyl Sulfide, 2-Methoxy-4-Propylphenol, 2-Methyl-Trans-2-Butenoic Acid, 4-(Methylthio)Butanol, 2-(Methylthio)Methyl-2-Butenal, 3-Octen-2-O1, 2-Octen-4-One, Octyl 2-Methylbutyrate, 3-Decanol, D-Xylose, Propyl 2-Methyl-3-Furyl Disulfide, 1-Hexen-3-O1, 2-Acetyl-5-Methylfuran, Epsilon-Dodecalactone, 2-Propionylthiazole, 1-Octen-3-Y1 Butyrate, Epsilon-Decalactone, 2-Propionylpyrrole, Thiazole, Benzenethiol, Benzyl Disulfide, 5-Phenylpentanol, 2-(2-Butyl)-4,5-Dimethyl-3-Thiazoline, 4,5-Dimethyl-2-Ethyl-3-Thiazoline, 4,5-Dimethyl-2-Isobutyl-3-Thiazoline, Delta-1-(2,6,6-Trimethyl-3-Cyclohexen-1-Y1)-2-Buten-1-One, 2-Ethyl-4-Hydroxy-5-Methyl-3(2h)-Furanone, Alpha-Ionol, Beta-Ionol Dihydro-Beta-Ionone, Dihydro-Beta-Ionol, Dihydro-Alpha-Ionone, 2-Methyl-4-Phenyl-2-Butanol, 4-Methyl-2-Pentyl-1,3-Dioxolan, Cyclohexylmethyl Pyrazine, Phenylethyl 2-Methylbutyrate, 3-Hexenyl Phenylacetate, 4,5-Dimethyl-3-Hydroxy-2,5-Dihydrofuran-2-One, 4-Hydroxy-5-Methyl-3(2h)-Furanone, 2-Methyl-3-Thioacetoxy-4,5-Dihydrofuran, 2-Trans-6-Cis-Dodecadienal, 2-Trans-4-Cis-7-Cis-Tridecatrienal, 2,6,6-Trimethyl-1&2-Cyclohexen-1-Carboxaldehyde, P-Methylcinnamaldehyde, Ethyl Trans-2-Decenoate, Ethyl Trans-4-Decenoate, Ethyl Trans-2-Octenoate, 2-Methylbutyl Acetate, Cis-5-Isopropenyl-Cis-2-Methylcyclopentan-1-Carboxaldehyde, 3-Methyl-2-Butenal, 3-Methyl-2-Buten-1-O1, Propyl 2,4-Decadienoate, P-Propylphenol, Butyl Salicylate, 6-Acetoxydihydrotheaspirane, 4-(P-Acetoxyphenyl)-2-Butanone, 4-Acetyl-6-T-Butyl -1,1-Dimethylindan, 4-Acetyl-2-Methylpyrimidine, 4-Allyl-2,6-Dimethoxyphenol, L-Aspartic Acid, Campholene Acetate, 1,4-Cineole, Alpha-1-(2,6,6-Trimethyl-2-Cyclohexen-1-Y1)-2-Buten-1-One, 9-Decenoic Acid, Nerol Oxide, Dihydroxyacetophenone, 2,6-Dimethyl-6-Hepten-1-O1, 2,5-Dimethyl-4-Methoxy-3(2h)-Furanone, 2,2-Dimethyl-5-(Methylpropen-1-Y1)Tetrahydrofuran, 2,6-Dimethylthiophenol, Diphenyl Ether, Disodium 5-Guanylate, Disodium 5-Inosinate, Trans,Trans-2,4-Dodecadienal, 4-Ethyl-2,6-Dimethoxyphenol, 2-Ethyl-4,5-Dimethyloxazole, 2-Ethylfuran, Ethyl 3-(Furfurylthio)Propionate, Ethyl Trans-2-Hexenoate, 1-Ethylhexyl Tiglate, Ethyl 3-Mercaptopropionate, Ethyl 2-Methyl-3,4-Pentadienoate, Ethyl 3-Methylpentanoate, 2-Ethyl-4-Methylthiazole, Ethyl 4-(Methylthio)Butyrate, Ethyl Cis-4,7-Octadienoate, Ethyl 3-Oxohexanoate, L-Glutamine, Glyceryl 5-Hydroxydecanoate, Glyceryl 5-Hydroxydodecanoate, Guaiacyl Acetate, Cis-3-Hexenyl Benzoate, Cis-3-Hexenyl Cis-3-Hexenoate, Cis-3-Hexenyl Lactate, Hexyl Benzoate, Hexyl Trans-2-Hexenoate, Hexyl 2-Methyl-3&4-Pentenoate, L-Histidine, Hydroquinone Monoethyl Ether, 5-Hydroxy-2,4-Decadienoic Acid Delta-Lactone, 2-Hydroxy-4-Methylbenzaldehyde, Isoeugenyl Benzyl Ether, Isopropyl 2-Methylbutyrate, 1-P-Menthene-8-Thiol, Methyl 1-Acetoxycyclohexyl Ketone, Methylbenzyl Acetate (Mixed O,M,P), 3-Methyl-2-Butanol, 4-Methyl-2,6-Dimethoxyphenol, 2-Methyl-1,3-Dithiolane, Methyl 2-Hydroxy-4-Methylpentanoate, Methyl 2-Methylpentanoate, Methyl 2-Methylthiobutyrate, Methyl Nicotinate, Methyl 3-Nonenoate, 2-Methyl-2-Octenal, Methyl Trans-2-Octenoate, Methyl 2-Oxo-3-Methylpentanoate, Methyl Sorbate, 7-Methyl-4,4a,5,6-Tetrahydro-2(3h)-Naphthalenone, 4-Methylthiazole, 2-(Methylthiomethyl)-3-Phenylpropenal, 3-Methyl-1,2,4-Trithiane, Beta-Naphthyl Isobutyl Ether, Cis-2-Nonen-1-O1, Trans,Trans-2,4-Octadienal, Cis-5-Octen-1-O1, 2-Oxobutyric Acid, 2-Pentadecanone, 2-Pentyl-1-Buten-3-One, D,L-Phenylalanine, 1-Phenyl-3 Or 5-Propylpyrazole, 4-Propenyl-2,6-Dimethoxyphenol, 4-Propyl-2,6-Dimethoxyphenol, L-Rhamnose, 1,2,5,6-Tetrahydrocuminic Acid, Thaumatin, P-Tolyl Octanoate, Salicylate, 2,2,6-Trimethyl-6-Vinyltetrahydropyran, L-Tyrosine, Vanillyl Alcohol, Vanillylidene Acetone, P-Vinylphenol, Anisyl Phenylacetate, Alpha-Campholenic Alcohol, 5- And 6-Decenoic Acid, 2,5-Diethyltetrahydrofuran, 5-Hydroxy-2-Decenoic Acid Delta-Lactone, 5-Hydroxy-7-Decenoic Acid Delta-Lactone, Linalool Oxide, Massoia Bark Oil (Cryptocarya Massoio), L-Menthyl Lactate, Cis-5-Octenal, Osmanthus Absolute (Osmanthus Fragrans Lour.), 2-(3-Phenylpropyl)Pyridine, Potassium 2-(1′-Ethoxy)Ethoxypropanoate, O-Tolyl Isobutyrate, Vanillin Isobutyrate, Dehydromenthofurolactone, 4-Ethylbenzaldehyde, Ethyl Methyl-P-Tolylglycidate, 5-Hydroxy-8-Undecenoic Acid Delta-Lactone, 5-Isopropenyl-2-Methyl-2-Vinyltetrahydrofuran, 1-(4-Methoxyphenyl)-4-Methyl-1-Penten-3-One, 5-Methyl-2-Hepten-4-One, 3-Methyl-1-Pentanol, 3-Methyl-2-(N-Pentanyl)-2-Cyclopenten-1-One, Mintlactone, Myrtenyl Acetate, 2-Trans-6-Trans-Nonadienal, 3-Oxodecanoic Acid Glyceride, 3-Oxododecanoic Acid Glyceride, 3-Oxohexadecanoic Acid Glyceride, 3-Oxohexanoic Acid Glyceride, 3-Oxooctanoic Acid Glyceride, 3-Oxotetradecanoic Acid Glyceride, Sodium 2-(4-Methoxyphenoxy)Propanoate, Theaspirane, Acetaldehyde Ethyl Cis-3-Hexenyl Acetal, Dihydronootkatone, 1-Ethoxy-3-Methyl-2-Butene, (Z)-3 & (E)-2-Hexenyl Propionate, Hydrogen Sulfide, 1,4-Dodec-6-Enolactone, 2 Or 4-Isobutyl-(4 Or 2),6-Dimethyldihydro-4h-1,3,5-Dithiazine, 2 Or 4-Isopropyl-(4 Or 2),6-Dimethyldihydro-4h-1,3,5-Dithiazine, Jambu Oleoresin, 3-L-Menthoxypropane-1,2-Diol, 4-Methoxy-2-Methyl-2-Butanethiol, Gamma-Methyldecalactone, 2-Methyl-3-Tetrahydrofuranthiol, Methylthio 2-(Acetyloxy)Propionate, 3-(Methylthio)Hexyl Acetate, Methylthio-2-(Propionyloxy)Propionate, Octahydrocoumarin, 2-Pentanethiol, D-Ribose, Sclareolide1,3,5-Undecatriene, Vanillyl Butyl Ether, 4-Acetoxy-2,5-Dimethyl-3(2h)Furanone, 2,4-Dihydroxybenzoic Acid, 1,2-Dimethoxybenzene, 4-Ethyloctanoic Acid, Ethyl Vanillin Beta-D-Glucopyranoside, 5-Hydroxy-2-Dodecenoic Acid Lactone, 4-Hydroxy-3-Methyloctanoic Acid Lactone, 2-Isopropyl-N,2,3-Trimethylbutyramide, L-Menthol Ethylene Glycol Carbonate, L-Menthol 1- And 2-Propylene Glycol Carbonate, L-Menthone 1,2-Glycerol Ketal, D,L-Menthone 1,2-Glycerol Ketal, Cis- And Trans-Menthone-8-Thioacetate, Mono-Menthyl Succinate, Neohesperidin Dihydrochalcone, Sodium 3-Methoxy-4-Hydroxycinnamate, Taurine, Thaumatin B—Recombinant, Vanillyl Ethyl Ether, 3-Acetylmercaptohexyl Acetate, 2-Acetyl-2-Thiazoline, D1-Alanine, L-Arginine, 1-Buten-1-Y1 Methyl Sulfide, Delta-3-Carene, Cycloionone, Daidai Peel Oil, 1-Decen-3-O1, Diethyl Sulfide, 2,5-Dihydroxy-1,4-Dithiane, Diisopropyl Disulfide, 2,4-Dimethylanisole, 2-(3,7-Dimethyl-2,6-Octadienyl)Cyclopentanone, (E,R)-3,7-Dimethyl-1,5,7-Octatrien-3-O1, 1,4-Dithiane, Ethyl 2,4,7-Decatrienoate, 2-Ethylhexanethiol, Ethyl 2-(Methyldithio)Propionate, Ethyl 2-(Methylthio)Acetate, Ethyl 3-(Methylthio)Butyrate, Ethyl Vanillin Isobutyrate, Ethyl Vanillin Propylene Glycol Acetal, Alpha-Farnesene, 4-[(2-Furamnethyl)Thio]-2-Pentanone, (Z)-4-Hepten-1-O1, 1-Hexanethiol, 3-Hydroxy-2-Oxopropionic Acid, Beta-Ionyl. Acetate, Alpha-Isomethylionyl Acetate, Litsea Cubeba Oil, L-Lysine, Cis- And Trans-P-1(7),8-Menthadien-2-Y1 Acetate, 3-(L-Menthoxy)-2-Methylpropane-1,2-Diol, 3-Mercaptohexanol, 3-Mercaptohexyl Acetate, 3-Mercaptohexyl Butyrate, 3-Mercaptohexyl Hexanoate, 3-Mercapto-3-Methyl-1-Butanol, 3-Mercapto-3-Methylbutyl Formate, 1-Mercapto-2-Propanone, S-Methyl Benzothioate, 3-Methylbutanethiol, Methyl (E)-2-(Z)-4-Decadienoate, Methyl Ethyl Sulfide, Methyl Ethyl Trisulfide, S-Methyl Hexanethioate, 2-(4-Methyl-2-Hydroxyphenyl)Propionic Acid-Gamma-Lactone, S-Methyl 3-Methylbutanethioate, Methyl 3-Methyl-1-Butenyl Disulfide, 2-Methyl-2-(Methyldithio)Propanal, S-Methyl 4-Methylpentanethioate, (E)-7-Methyl-3-Octen-2-One, 3-Methyl-2-Oxobutanoic Acid, 3-Methyl-2-Oxopentanoic Acid, 4-Methyl-2-Oxopentanoic Acid, Methyl Phenyl Disulfide, Methyl Phenyl Sulfide, 2-Methyl-1-Propanethiol, Methyisulfinylmethane, S-Methyl Thioacetate, 3-Methylthiohexanal, Bis-(Methylthio)Methane, Methylthiomethyl Butyrate, Methylthiomethyl Hexanoate, 4-(Methylthio)-2-Oxobutanoic Acid, 1-Methylthio-2-Propanone, 3-(Methylthio)Propyl Acetate, (E)-3-(Z)-6-Nonadien-1-O1, (Z)(Z)-3,6-Nonadien-1-O1, 8-Ocimenyl Acetate, (E)-2-Octen-1-O1, (E)-2-Octen-4-O1, (E)-2-(2-Octenyl)Cyclopentanone, (Z)-5-Octenyl Propionate, 2-Oxopentanedioic Acid, 2-Oxo-3-Phenylpropionic Acid, 2-Pentyl Butyrate, Phenylethyl Mercaptan, Prenyl Thioacetate, Prenylthiol, 2-Propanethiol, 1-Pyrroline, Sarcodactylis Oil, Sodium Diacetate, Sodium 3-Mercaptooxopropionate, Tea Tree Oil, 2,3,4-Trimethyl-3-Pentanot, Vanillin 3-(L-Menthoxy)Propane-1,2-Diol Acetal, Vanillin Propylene Glycol Acetal, 2-Aminoacetophenone, Bornyl Butyrate, (E)-2-Butenoic Acid, Cyclohexanone, Cyclopentanone, 2,4-Decadien-1-O1, 9-Decenal, 2-Decenoic Acid, 4-Decenoic Acid, 2,5-Diethyl-3-Methylpyrazine, 3,5-Diethyl-2-Methylpyrazine, 6,7-Dihydro-2,3-Dimethyl-5h-Cyclopentapyrazine, P-Tert-Butylphenol, 2-Ethyl-6-Methylpyrazine, (E)-2-Heptenoic Acid, 2,4-Hexadienoic Acid, (E,E)-, 2,4-Hexadien-1-O1, 3-Hexenal, (Z)-2-Hexen-1-O1, Cis-3-Hexenyl Anthranilate, Trans-2-Hexenyl Butyrate, (E)-2-Hexenyl Formate, 3-Hexenyl 2-Hexenoate, Cis-3-Hexenyl Isobutyrate, Trans-2-Hexenyl Isovalerate, Cis-3-Hexenyl Tiglate, Trans-2-Hexenyl Propionate, Cis-3-Hexenyl Propionate, 3-Hexenyl 2-Oxopropionate, Trans-2-Hexenyl Pentanoate, Cis-3-Hexenyl Valerate, 5-(Cis-3-Hexenyl)Dihydro-5-Methyl-2(3h)Furanone, 4-Isopropyl-2-Cyclohexenone, 2-Isopropylpyrazine, 2-Methyl-3-(1-Oxopropoxy)-4h-Pyran-4-one, Mesquite Wood Extract, 2-Methoxybenzoic Acid, 3-Methoxybenzoic Acid, 4-Methoxybenzoic Acid, 2-Methylcyclohexanone, 3-Methylcyclohexanone, 4-Methylcyclohexanone, 2-Methyl-3-(Methylthio)Furan, Michelia Alba Oil, 2,4-Nonadien-1-O1, (E,Z)-2,6-Nonadien-1-O1 Acetate, (E,Z)-3,6-Nonadien-1-O1 Acetate, (E)-2-Nonenoic Acid, 3-Nonen-2-One, (E,E)-2,4-Octadien-1-O1, (E)-2-Octenoic Acid, Phenyl Acetate, 2-Phenylphenol, Phenyl Salicylate, Propylpyrazine, 3,5,5-Trimethylcyclohex anol, 2,3,6-Trimethylphenol, 2-Acetyl-3-Methylpyrazine, 1-Amino-2-Propanol, 3-Decanone, Cis-4-Decenyl Acetate, Diisopropyl Trisulfide, (E) & (Z)-4,8-Dimethyl-3,7-Nonadien-2-One, 2,5-Dimethyl-3-Oxo-(2h)-Fur-4-Y1 Butyrate, Cis And Trans-2,5-Dimethyltetrahydrofuran-3-Thiol, Cis And Trans-2,5-Dimethyltetrahydro-3-Furyl Thioacetate, Ethanethioic Acid, S-(2-Methyl-3-Furanyl) Ester, Ethyl 4-(Acetylthio)Butyrate, Ethyl Cis-4-Heptenoate, Ethyl 5-Hexenoate, (+/−)-Ethyl 3-Mercaptobutyrate, Ethyl 5-(Methylthio)Valerate, Furfuryl Propyl Disulfide, (+/−)-Heptan-3-Y1 Acetate, (+/−)-Heptan-2-Y1 Butyrate, (Z)-3-Hexenyl (E)-2-Butenoate, (E)-2-Hexenyl Hexanoate, 4-Hydroxybenzaldehyde, 2-Hydroxybenzoic Acid, 4-Hydroxybenzoic Acid, 4-Hydroxybenzyl Alcohol, 4-Hydroxy-3-Methoxybenzoic Acid, 3(2)-Hydroxy-5-Methyl-2(3)-Hexanone, Isopentylidene Isopentylamine, Isoprenyl Acetate, D,L-Menthol(+/−)-Propylene Glycol Carbonate, Erythro And Threo-3-Mercapto-2-Methylbutan-1-O1, 3-Mercapto-2-Methylpentanal, (+/−)-2-Mercapto-2-Methylpentan-1-O1, 3-Mercapto-2-Methylpentan-1-O1 (Racemic), 4-Mercapto-4-Methyl-2-Pentanone, (+/−)-2-Methyl-1-Butanol, (+/−)-3-Methyl-Gamma-Decalactone, 2-Methylheptan-3-One, (E)-6-Methyl-3-Hepten-2-One, Methyl 2-Methy2-Propenoate, Methyl (Methylthio)Acetate, 2-(Methylthio)Ethanol, 12-Methyltridecanal, L-Monomenthyl Glutarate, (+/−)-Nonan-3-Y1 Acetate, (E,E)-3,5-Octadien-2-One, (+/−)-Octan-3-Y1 Formate, Paraldehyde, 4-Pentenyl Acetate, 2-Pentyl Acetate, Perilla Leaf Oil, Phenethyl Isothiocyanate, Pyrazine, Sodium 4-Methoxybenzoyloxyacetate, 2,4,6-Triisobutyl-5,6-Dihydro-4h-1,3,5-Dithiazine, 2,4,6-Trimethyldihydro-4h-1,3,5-Dithiazine, 3,7,11-Trimethyl-2,6,10-Dodecatrienal, (+/−)-(2,6,6-Trimethyl-2-Hydroxycyclohexylidene)Acetic Acid Gamma-Lactone, Trithiahexane-2,3,5,6-Undecanone, Vanillin Erythro And Threo-Butan-2,3-Diol Acetal, Acetaldehyde Diisoamyl Acetal, Amyl Methyl Disulfide, Benzyl Hexanoate, Butyl Ethyl Disulfide, Beta-Cyclodextrin, Diethyl Trisulfide, (+/−)-3,5-Diethyl-1,2,4-Trithiolane, (+/−)-Dihydrofarnesol, Dihydromintlactone, Dihydroxyacetone, 2,5-Dimethyl-3-Furanthiol Acetate, 2,5-Dimethylthiazole, (Z)-4-Dodecenal, 4,5-Epoxy-(E)-2-Decenal, Ethyl 3-Acetoxy-2-Methyl Butyrate, S-Ethyl 2-Acetylamino Ethanethioate, Ethyl Methyl Disulfide, Ethyl Propyl Disulfide, Ethyl Propyl Trisulfide, O-Ethyl S-(2-Furylmethyl)Thiocarbonate, Geranyl Tiglate, Grape Seed Extract, Trans-4-Hexenal, (E)-2-Hexenal Diethyl Acetal, 2-Hexyl-4,5-Dimethyl-1,3-Dioxolane, 4-Hydroxy-3,5,-Dimethoxy Benzaldehyde, 4-Hydroxy-2,3-Dimethyl-2,4-Nonadienoic Acid Gamma Lactone, 4-Hydroxy-4-Methyl-5-Hexenoic Acid Gamma Lactone, 3-Hydroxy-4-Phenylbutan-2-One, P-Menthane-3,8-Diol, L-Menthyl Methylether, Methyl 5-Acetoxyhexanoate, 3-[(2-Methyl-3-Furyl)Thio]-2-Butanone, 3-Methyl-2,4-Nonanedione, (+/−)-2-(5-Methyl-5-Vinyl-Tetrahydrofuran-2-Y1)Propionaldehyde, 9-Octadecenal, 2,3-Octanedione, (+/−)-1-Phenylethylmercaptan, 4-Propenylphenol, 2-Propionylpyrroline, 2-Propionyl-2-Thiazoline, 2-Propylpyridine, (Z)-8-Tetradecenal, Tuberose Lactone, 2-Undecen-1-O1, (+/−)-1-Acetoxy-1-Ethoxyethane, 4-Acetyl-2,5-Dimethyl-3(2h)-Furanone, 2-Acetyl-3,5-Dimethylfuran, Allyl Crotonate, Allyl Propyl Disulfide, Allyl Valerate, 4-Allylphenol, Allyl Thiohexanoate, O-Anisaldehyde, N-Benzoylanthranilic Acid, Thujyl Alcohol, L-Bornyl Acetate, 2-Butylfuran, Butyl Isothiocyanate, 2-Butyrylfuran, Carvone-5,6-Oxide, Beta-Caryophyllene Oxide, Citronellyl Anthranilate, N-Cyclopropyl-Trans-2-Cis-6-Nonadienamide, Trans-Alpha-Damascone, 2,4,7-Decatrienal, 2-Decylfuran, Dehydronootkatone, Diacetyl Tartaric Acid Esters Of Mono- And Diglycerides, Diethyl Disulfide, Mixture Of 3,6-Diethyl-1,2,4,5-Tetrath iane And 3,5-Diethyl-1,2,4-Trithiolane, 2,4-Difurfurylfuran, Diisopentyl Thiomalate, Dimercaptomethane, 1,1-Dimethoxy-Trans-2-Hexene, 2,4-Dimethyl-1,3-Dioxolane, 3,5- And 3,6-Dimethyl-2-Isobutylpyrazine, 2,5-Dimethyl-3(2h)-Furanone, (+/−)-Trans- And Cis-4,8-Dimethyl-3,7-Nonadien-2-O1, Trans- And Cis-4,8-Dimethyl-3,7-Nonadien-2-Y1 Acetate, 2,5-Dimethyl-4-Ethoxy-3(2h)-Furanone, (+/−)-Trans- And Cis-5-(2,2-Dimethylcyclopropyl)-3-Methyl-2-Pentenal, 2,5-Dimethylfuran, Divanillin, (+/−)-2,8-Epithio-Cis-P-Menthane, Epoxyoxophorone, Tomato Lycopene, Tomato Powder, Dehydrated Tomato, Tomato Extract, Tomato Juice, Tomato, Ethane-1,1-Dithiol, Ethyl Cis-3-Hexenoate, N-Ethyl Trans-2-Cis-6-Nonadienamide, Ethyl Furfuryl Ether, Ethyl N-Ethylanthranilate, Ethyl N-Methylanthranilate, (+/−)-4-Ethyloctanal, Eugenyl Isovalerate, Furfuryl 2-Methyl-3-Furyl Disulfide, 1-(2-Furyl)Butan-3-one, Geranic Acid, Geranyl 2-Methylbutyrate, Geranyl Valerate, Glyceryl-Lacto Esters Of Fatty Acids, Hept-Trans-2-En-1-Y1 Acetate, Hept-2-En-1-Y1 Isovalerate, Trans-2-Trans-4-Heptadien-1-O 1, 2-Heptanethiol, (+/−)-1-Hepten-3-O1, Cis-And Trans-2-Heptylcyclopropanecarboxylic Acid, 2,4-Hexadienyl Propionate, 2,4-Hexadienyl Acetate, 2,4-Hexadienyl Butyrate, 2,4-Hexadienyl Isobutyrate, 2-Hexenyl Octanoate, Hexyl 3-Mercaptobutanoate, 2-Hexylthiophene, 4-Hydroxy-2-Butenoic Acid Gamma-Lactone, 3-Hydroxy-2-Octanone, 2-(2-Hydroxy-4-Methyl-3-Cyclohexenyl)Propionic Acid Gamma-Lactone, 5-Hydroxy-4-Methylhexanoic Acid Delta-Lactone, 1-(3-Hydroxy-5-Methyl-2-Thienyl)Ethanone, (+/−)-2-Hydroxypiperitone, Beta-Ionone Epoxide, Isoambrettolide, Isobornyl Isobutyrate, Isobornyl 2-Methylbutyrate, N-Isobutyldeca-Trans-2-Trans-4-Dienamide, Isobutyl N-Methylanthranilate, (+/−)-Isobutyl 3-Methylthiobutyrate, Beta-Isomethylionone, Isopropenyl Acetate, Lactylated Fatty Acid Esters Of Glycerol And Propylene Glycol, 2-(L-Menthoxy)Ethanol, Menthyl Pyrrolidone Carboxyl ate, Menthyl Valerate, 4-Mercapto-2-Pentanone, (+/−)-4-Mercapto-4-Methyl-2-Pentanol, 2-Mercaptoanisole, Methionyl Butyrate, Trans- And Cis-1-Methoxy-1-Decene, (S 1)-Methoxy-3-Heptanethiol, 2-Methoxyacetophenone, Methyl Cis-3-Hexenoate, Methyl Cis-5-Octenoate, Methyl 3-(Methylthio)Butanoate, Methyl 3-Mercaptobutanoate, Methyl Isopentyl Disulfide, Methyl N,N-Dimethylanthranilate, Methyl N-Acetylanthranilate, Methyl N-Formylanthranilate, S-Methyl Propanethioate, 2-Methyl-1-Methylthio-2-Butene, 3-Methyl-2(3-Methylbut-2-En-1-Y1)Furan, 3-(5-Methyl-2-Furyl)Prop-2-Enal, 5-Methyl-3(2h)-Furanone, 6-Methyl-5-Hepten-2-Y1 Acetate, 2-Methylbut-2-En-1-O1, 2-Methylfuran, 4-Methylpent-2-Enoic Acid, 3-(Methylthio)-2-Butanone, 4-(Methylthio)-2-Pentanone, (+/−)-3-(Methylthio)Heptanal, 3-(Methylthio)Methylthiophene, Methylthiomethylmercaptan, Mono- And Diglycerides Of Fatty Acids, Nona-2,4,6-Trienal, 2-Nonenoic Acid Gamma-Lactone, Cis-3-Octenyl Propionate, L-Ornithine Monochlorohydrate/Ornithine, Pent-2-Enyl Hexanoate, 2-Pentanoylfuran, 2-Pentenoic Acid, (+/−)-2-Phenyl-4-Methyl-2-Hexenal, Phthalide, Phytol, Phytyl Acetate, 3-Pinanone, Piperitenone Oxide, L-Piperitone, Polyglycerol Esters Of Fatty Acids, Prenyl Acetate, Prenyl Benzoate, Prenyl Caproate, Prenyl Formate, Prenyl Isobutyrate, Propyl 2-Mercaptopropionate, Propylene Glycol Mono- And Diesters Of Fatty Acids, Tetradec-2-Enal, Thioacetic Acid, Trans- And Cis-2,4,8-Trimethyl-3,7-Nonadien-2-O1, (+/−)-2,4,8-Trimethyl-7-Nonen-2-O1, 3,7,11-Trimethyldodeca-2,6,10-Trienyl Acetate, 2,4,6-Trithiaheptane, Tyramine, Verbenone, Vetiverol, Vetiveryl Acetate, Commint Oil, Mentha Arvensis L., Heliopsis Longipes Extract, Scotch Spearmint Oil, Mentha Cardiaca L., Natural Hickory Smoke Flavor, Betaine, Adenosine Monophosphate; Monosodium, Or Disodium Adenylate, Isoquercitrin, Enzymatically Modified, Glycerol Ester Of Rosin, Gum Arabic, Hydrogen Octenylbutane Dioate, (−)-Homoeriodictyol, Sodium Salt, Sugar Beet Juice Extract, (+/−)-N,N-Dimethyl Menthyl Succinamide, Ni-(2-Methoxy-4-Methylbenzyl)-N2-(2-(Pyridin-2Y1)Ethyl)Oxalamide, N-(Heptan-4-Y1)Benzo[D][1,3]Dioxole-5-Carboxamide, N1-(2,4-Dimethoxybenzyl)-N2-(2-(Pyridin-2-Y1)Ethyl)Oxalamide, N1-(2-Methoxy-4-Methylbenzyl)-N2-(2-(5-Methylpyridin-2-Y1)Ethyl)Oxatamide, 1,6-Hexalactam, Ethylamine, Propylamine, Isopropylamine, Isobutylamine, Sec-Butylamine, 2-Methylbutylamine, Pentylamine, Hexylamine, 2-Methylpiperidine, Trimethylamine Oxide, Triethylamine, Tripropylamine, N,N-Dimethylphenethylamine, 2-Acetyl-1-Pyrroline, Piperazine, Butyramide, Methyl 10-Undecenoate, N-Gluconyl Ethanolamine, N-Gluconyl Ethanolamine Phosphate, N-Lactoyl Ethanolamine, N-Lactoyl Ethanolamine Phosphate, Ethanethiol, Heptane-1-Thiol, S-Isopropyl 3-Methylbut-2-Enethioate, 3-Methylhexanal, 4-Pentenal, Propyl Propane Thiosulfonate, Alpha-Ionene, Gardenia Gummifera Distillate, Piper Longum Distillate, N-3,7-Dimethyl-2,6-Octadienylcyclopropylcarboxamide, (+/−)-Ethyl 2-Hydroxy-2-Methylbutyrate, (+/−)-Ethyl 2-Hydroxy-3-Methylvalerate, 2-(2-Hydroxyphenyl) Cyclopropanecarboxylic Acid Delta Lactone, 2-Decanone, (+/−)-Trans- And Cis-2-Hexenal Propylene Glycol Acetal, (+/−)-Trans- And Cis-2-Hexenal Glyceryl Acetal, Trans-2-Hexenyl 2-Methylbutyrate, 2-(4-Methyl-5-Thiazolyl)Ethyl Formate, 2-(4-Methyl-5-Thiazolyl)Ethyl Propionate, 2-(4-Methyl-5-Thiazolyl)Ethyl Butanoate, 2-(4-Methyl-5-Thiazolyl)Ethyl Isobutyrate, 2-(4-Methyl-5-Thiazolyl)Ethyl Hexanoate, 2-(4-Methyl-5-Thiazolyl)Ethyl Octanoate, 2-(4-Methyl-5-Thiazolyl)Ethyl Decanoate, (+/−)-3-(Ethylthio)Butanol, Decalepis Hamiltonii Extract, 2-(Trans-2-Pentenyl)Cyclopentanone, 3,9-Dimethyl-6-(1-Methylethyl)-1,4-Dioxaspiro[4.5]Decan-2-One, Cis- And Trans-2-Isobutyl-4-Methyl-1,3-Dioxolane, Cis-And Trans-2-Isopropyl-4-Methyl-1,3-Dioxotane, 4-Aminobutyric Acid, 3-Mercaptoheptyl Acetate, Ethyl Trans-2-Methyl-2-Pentenoate, Methyl Hexyl Ether, Trans-2-Trans-4-Nonadiene, 1-Octene, Cis- And Trans-Ethyl 2,4-Dimethyl-1,3-Dioxolane-2-Acetate, Citronellyl Trans-2-Methyl-2-Butenoate, 5-Acetyl-2,3-Dihydro-1,4-Thiazine, Bis(1-Mercaptopropyl)Sulfide, 2,5-Dithiahexane, Pseudoionone, Cis- And Trans-L-Mercapto-P-Menthan-3-One, Trans-2-Nonen-4-One, Trans-4-Nonenal, 1,1′-(Tetrahydro-6a-Hydroxy-2,3a,5-Trimethylfuro[2,3-D]-1,3-Dioxole-2,5-Diyl)Bis-Ethanone, Trans-2-Decenol, Cis-2-Pentenol, 2-Methylbutyl 3-Methyl-2-Butenoate, Citric And Fatty Acid Esters Of Glycerol, L-Menthyl (R,S)-3-Hydroxybutyrate, N-(Ethoxycarbonyl)Methyl)-P-Menthane-3-Carboxamide, N-[2-(3,4-Dimethoxyphenyl)Ethyl]-3,4-Dimethoxycinnamic Acid Amide, Mixture Of Methyl Cyclohexadiene And Methylene Cyclohexene, (+/−)-Cis- And Trans-1,2-Dihydroperillaldehyde, 5,7-Dihydroxy-2-(3-Hydroxy-4-Methoxyphenyl)Chroman-4-One, Phenethyl Decanoate, 3,6-Dimethyl-2,3,3a,4,5,7a-Hexahydrobenzofuran, 2-Methylacetophenone, 1-Ethyl-2-Pyrrolecarboxaldehyde, Cis- And Trans-5-Ethyl-2,5-Dihydro-4-Methyl-2-(1-Methylpropyl)-Thiazole, Cis And Trans-5-Ethyl-4-Methyl-2-(2-Methylpropyl)-Thiazoline, 2-Methyl-3-Furyl Methylthiomethyl Disulfide, Pyrrolidino-[1,2e]-4h-2,4-Dimethyl-1,3,5-Dithiazine, S-Allyl-L-Cysteine, 5-Pentyl-3h-Furan-2-One, 3-Mercapto-3-Methyl-1-Butyl Acetate, (+/−)-3-Mercapto-1-Butyl Acetate, 5-Nonen-Trans-2-One, L-Menthyl Acetoacetate, 4-Octen-3-One, 2,4,6-Trimethylphenol, 4-Hydroxyacetophenone, (+/−)-([R-(E)]-5-Isopropyl-8-Methylnona-6,8-Dien-2-One, 1-Methyl-1h-Pyrrole-2-Carboxaldehyde, 1-Pentanethiol, Pentadecanoic Acid, Tridecanal, Tridecanoic Acid, Hexyl Heptanoate, Dodecyl Propionate, Hexyl Nonanoate, Dodecyl Butyrate, Heptyl Heptanoate, Hexyl Decanoate, Ethyl 4-Methylpentanoate, Ethyl 2-Ethylbutyrate, Ethyl 2-Ethylhexanoate, 5-Methylhexyl Acetate, 4-Methylpentyl Isovalerate, 3,7-Dimethyloctanal, Cis-4-Decenol, Cis-5-Octenoic Acid, 5-Hexenol, 3-Isopropenylpentanedioic Acid, Methyl 4-Pentenoate, Cis-4-Octenol, 11-Dodecenoic Acid, Trans-3-Hexenol, Trans-4-Octenoic Acid, Isobutyl 10-Undecenoate, Cis-9-Octadecenyl Acetate, Ethyl 4-Pentenoate, Ethyl 3-Octenoate, 3-Octenoic Acid, Cis-9-Octadecenol, Decanal Propyleneglycol Acetal, Acetaldehyde Hexyl Isoamyl Acetal, Dodecanal Dimethyl Acetal, Nonanal Dimethyl Acetal, Heptanal Propyleneglycol Acetal, Hexanal Hexyl Isoamyl Acetal, Hexanal Dihexyl Acetal, Isovaleraldehyde Diethyl Acetal, Valeraldehyde Propyleneglycol Acetal, Nonanal Propyleneglycol Acetal, Undecanal Propyleneglycol Acetal, Valeraldehyde Dibutyl Acetal, Acetaldehyde 1,3-Octanediol Acetal, Hexanal Octane-1,3-Diol Acetal, Isovaleraldehyde Glyceryl Acetal, Acetaldehyde Di-Cis-3-Hexenyl Acetal, 2,6-Dimethyl-5-Heptenal Propyleneglycol Acetal, Octanal Propyleneglycol Acetal, Hexanal Butane-2,3-Diol Acetal, Pecan Shell Flour, Di-(1-Propenyl)-Sulfide (Mixture Of Isomers), 2-Pentylthiophene, 5-Ethyl-2-Methylthiazole, 2,4-Dimethylpyridine, 3-(4-Hydroxyphenyl)-1-(2,4,6-Trihydroxyphenyl)Propan-1-One, (+/−)-Ethyl 3-Hydroxy-2-Methylbutyrate, (+/−)-Ethyl3-Mercapto-2-Methylbutanoate, (+/−)-Cis- And Trans-2-Methyl-2-(4-Methyl-3-Pentenyl)Cyclopropanecarbaldehyde, Trimethyloxazole, 2,5-Dimethyl-4-Ethyloxazole, 2-Propyl-4,5-Dimethyloxazole, 2-Isobutyl-4,5-Dimethyloxazole, 2-Methyl-4,5-Benzoxazole, 2-Nonanone Propyleneglycol Acetal, 6-Methyl-5-Hepten-2-One Propyleneglycol Acetal, 2-Pentyl 2-Methylpentanoate, 3-Octyl Butyrate, Dimethylbenzyl Carbinyl Crotonate, Dimethylbenzyl Carbinyl Hexanoate, 1,5-Octadien-3-One, 10-Undecen-2-One, 2,4-Dimethyl-4-Nonanol, 8-Nonen-2-One, 8-P-Menthene-1,2-Diol, Caryophyllene Alcohol, D-2,8-P-Menthadien-1-O1, Cis-3-Nonen-1-O1, Trans-3-Hexenyl Acetate, 4-(Methylthio)Butyl Isothiocyanate, 6-(Methylthio)Hexyl Isothiocyanate, 5-(Methylthio)Pentyl Isothiocyanate, Amyl Isothiocyanate, 3-Butenyl Isothiocyanate, 2-Butylisothiocyanate, Ethyl Isothiocyanate, 5-Hexenyl Isothiocyanate, Hexyl Isothiocyanate, Isoamyl Isothiocyanate, Isobutyl Isothiocyanate, Isopropyl Isothiocyanate, Methyl Isothiocyanate, 4-Pentenyl Isothiocyanate, Benzyl Isothiocyanate, 2,4-Dimethyl-3-Oxazoline, 3,4-Dihydroxybenzoic Acid, 3-Hydroxybenzoic Acid, (+/−)-Acetaldehyde Ethyl Isopropyl Acetal, (+/−)-6-Methyloctanal, 5-Ethyl-2,3-Dimethylpyrazine, 2-Hydroxy-4-Methoxybenzaldehyde, 3-(Methylthio)Propyl Hexanoate, Sodium Lauryl Sulfate, Beta-Angelicalactone, 7-Decen-4-Olide, 9-Decen-5-Olide, 8-Decen-5-Olide, 6[5(6)-Decenoyloxy]Decanoic Acid, Ethyl 5-Acetoxyoctanoate, Ethyl 5-Hydroxydecanoate, 9-Dodecen-5-Olide, Gamma-Octadecalactone, Delta-Octadecalactone, 9-Tetradecen-5-Olide, Orin Lactone, Methyl 3-Hydroxybutyrate, Methyl 3-Acetoxy-2-Methylbutyrate, Ethyl 2-Acetylhexanoate, Ethyl 3-Hydroxyoctanoate, Methyl 3-Acetoxyoctanoate, 5-Oxooctanoic Acid, 5-Oxodecanoic Acid, Ethyl 5-Oxodecanoate, 5-Oxododecanoic Acid, Ethyl 2-Acetyloctanoate, 2-Oxo-3-Ethyl-4-Butanolide, 3-Isopropenyl-6-Oxoheptanoic Acid, Hydroxyacetone, 1-Hydroxy-4-Methyl-2-Pentanone, Propyleneglycol Diacetate, Propyleneglycol Dipropionate, Propyleneglycol Dibutyrate, Propyleneglycol Mono-2-Methylbutyrate, Propyleneglycol Di-2-Methylbutyrate, Propyleneglycol Monohexanoate, Propyleneglycol Dihexanoate, Propyleneglycol Dioctanoate, Dimethyl Adipate, Dipropyl Adipate, Diisopropyl Adipate, Diisobutyl Adipate, Dioctyl Adipate, Ethyl Acetoacetate Ethyleneglycol Ketal, Methyl Levulinate, Ethyl Levulinate Propyleneglycol Ketal, Propyl Levitate, Isoamyl Levitate, Dodecyl Lactate, Hexadecyl Lactate, Propyl Pyruvate, Hydroxycitronellal Propyleneglycol Acetal, Citral Glyceryl Acetal, Mushroom Oil, Distilled, Propyleneglycol Monobutyrate, Cis-3-Hexenyl Acetoacetate, 2-Methoxy-6-(2-Propenyl)Phenol, Myricitrin, (R)-(−)-1-Octen-3-O1, Cis-3-Hexenoic Acid, Ammonia (Also Includes Ammonium Chloride), Naringin Dihydrochalcone, N-P-Benzeneacetonitrilementhanecarboxamide, Cubebol, 6-Methytheptanal, (+/−)-Cis- And Trans-2-Pentyl-4-Propyl-1,3-Oxathiane, Choline Chloride (Also Includes Choline), 3-[(2-Methyl-3-Furyl)Thio]Butanal, (−)-Sclareol, (+)-Cedrol, D-Limonen-10-O1, (2,4)- And (3,5)-And (3,6)-Dimethyl-3-Cyclohexenylcarbaldehyde, 1,3-P-Menthadien-7-A1, P-Menthan-7-O1, P-Menth-1-En-9-O1, Menthyl Formate, Menthyl Propionate, Cyclotene Propionate, 3,3,5-Trimethylcyclohexyl Acetate, D1-Camphor, 2-Cyclopentylcyclopentanone, Caryl Palmitate, Cyclohexanone Diethyl Ketal, 2-Cyclohexenone, 8,9-Dehydrotheaspirone, L-Fenchone, Ethylenediaminetetraacetic Acid Disodium Salt, 2,2,6,7-Tetramethylbicyclo[4.3.0]Nona-4,9(1)-Dien-8-O1, 2,2,6,7-Tetramethylbicyclo[4.3.0]Nona-4,9(1)-Dien-8-One, 6-Hydroxycarvone, L-Menthyl Butyrate, Pinocarvyl Isobutyrate, 2-Pentenyl-4-Propyl-1,3-Oxathiane (Mixture Of Isomers), Acetaldehyde Di-Isobutylacetal, Acetaldehyde Ethyl Isobutyl Acetal, 4-(2,2,3-Trimethylcyclopentyl)Butanoic Acid, Perillaldehyde Propyleneglycol Acetal, 2,6,6-Trimethyl-2-Hydroxycyclohexanone, Acetoin Propyleneglycol Ketal, 4,5-Octanedione, Ethyl Maltol Isobutyrate, 2-Tetrahydrofurfuryl 2-Mercaptopropionate, Nerolidol Oxide, Furfural Propyleneglycol Acetal, Methyl 3-(Furfurylthio)Propionate, Furfuryl Decanoate, Di-2-Furylmethane, (E)-Ethyl 3-(2-Furyl)Acrylate, Furfuryl Formate, 2-Methylbenzofuran, 5-Methylfurfuryl Alcohol, 2-Methyl-3-Furyl 2-Methyl-3-Tetrahydrofuryl Disulfide, Ethyl 2,5-Dimethyl-3-Oxo-4(2h)-Furyl Carbonate, Acai Berry Extract, 4-(2-Propenyl)Phenyl-Beta-D-Glucopyranoside, N-(2-(Pyridin-2-Y1)Ethyl)-3-P-Menthanecarboxamide, (+/−)-N-Lactoyl Tyramine, Cis,Cis-3,6-Nonadienyl Acetate, Trans-2-Nonenyl Acetate, Cis-3-Nonenyl Acetate, Cis-6-Nonenyl Acetate, Dihydrogalangal Acetate, 2,3,3-Trimethylindanone, N-Ethyl-2,2-Diisopropylbutanamide, Cyclopropanecarboxylic Acid (2-Isopropyl-5-Methylcyclohexyl)Amide, Magnolol, 2-(Methylthio)Ethyl Acetate, 3-(Methylthio)Propyl Mercaptoacetate, Ethyl 2-Hydroxyethyl Sulfide, Ethyl 3-(Methylthio)-Cis-2-Propenoate, Ethyl 3-(Methylthio)-Trans-2-Propenoate, Ethyl 3-(Methylthio)-2-Propenoate, 4-Methyl-2-(Methylthiomethyl)-2-Hexenal, 5-Methyl-2-(Methylthiomethyl)-2-Hexenal, 4-Methyl-2-(Methylthiomethyl)-2-Pentenal, 1-(3-(Methylthio)-Butyryl)-2,6,6-Trimethylcyclohexene, 2-Oxothiolane, Butyl Beta-(Methylthio)Acrylate, Ethyl 3-(Ethylthio)Butyrate, Methyl Octyl Sulfide, Methyl 1-Propenyl Sulfide, Diisoamyl Disulfide, Bis(2-Methylphenyl) Disulfide, Mixture Of Butyl Propyl Disulfide And Propyl And Butyl Disulfide, Di-Sec-Butyl Disulfide, Methyl 2-Methylphenyl Disulfide, Diisoamyl Trisulfide, Dodecanethiol, 2-Hydroxyethanethiol, 4-Mercapto-4-Methyl-2-Hexanone, 3-Mercapto-3-Methylbutyl Isovalerate, 3-Mercaptohexanal, Methyl Isobutanethioate, 3-Mercaptopropionic Acid, 2-Ethylhexyl 3-Mercaptopropionate, Butanal Dibenzyl Thioacetal, Methional Diethyl Acetal, Ethyl Linalyl Ether, Myrcenyl Methyl Ether, Linalool Oxide Pyramid, 2-Hydroxy-5-Methylacetophenone, 2-Phenylpropanal Propyleneglycol Acetal, Cinnamaldehyde Propyleneglycol Acetal, Ethyl Alpha-Acetylcinnamate, Ethyl 2-Hydroxy-3-Phenylpropionate, 3-(3,4-Methylenedioxyphenyl)-2-Methylpropanal, Trehalose, Dihydrate, Rebaudioside A, N-(2-Hydroxyethyl)-2,3-Dimethyl-2-Isopropylbutanamide, N-(11-Dimethyl-2-Hydroxyethyl)-2,2-Diethylbutanamide, Dimenthyl Glutarate, Trans-3-Nonen-1-O1, 4-Formyl-2-Methoxyphenyl 2-Hydroxypropanoate, Guaiacol Butyrate, Guaiacol Isobutyrate, Guaiacol Propionate, Ethyl 5-Hydroxyoctanoate, Isopropylideneglyceryl 5-Hydroxydecanoate, 2-Ethyl-2-Hexenal, Ethyl 2-Hexenoate, Propyl Sorbate, Cis-2-Octenol, 2-Hexylidenehexanal, Trans-2-Tridecenol, 2-Phenoxyethyl Propionate, Propyl 4-Tert-Butylphenylacetate, 2-Phenoxyethanol, Phenyl Butyrate, Piperonal Propyleneglycol Acetal, Benzyl Levitate, 4-Methylbenzyl Alcohol, Phenylacetaldehyde Diethyl Acetal, Benzyl Nonanoate, Anisaldehyde Propyleneglycol Acetal, 4-Methylbenzaldehyde Propyleneglycol Acetal, Phenylacetaldehyde Propyleneglycol Acetal, 2-Ethylhexyl Benzoate, 2-Ethyl-3-Methylthiopyrazine, 2-Ethoxy-3-Isopropylpyrazine, 2-Ethoxy-3-Ethylpyrazine, Butyl Beta-Naphthyl Ether, Isoamyl Phenethyl Ether, 2-Acetyl-4-Isopropenylpyridine, 4-Acetyl-2-Isopropenylpyridine, 2-Acetyl-4-Isopropylpyridine, 2-Methoxypyridine, 6-Methoxyquinoline, 2-Pentylthiazole, 2-Thienylmethanol, 2-Acetyl-5-Methylthiophene, 4-Methyl-3-Thiazoline, 3,4-Dimethylthiophene, 1-(2-Thienyl)Ethanethiol, 4,5-Dimethyl-2-Isobutylthiazole, Cyclotene Butyrate, 3-(Methylthio)Propylamine, 4-Methyl-Cis-2-Pentene, 1-Nonene, 1,3,5,7-Undecatetraene, Ethyl Alpha-Ethyl-Beta-Methyl-Beta-Phenylglycidate, Methyl Beta-Phenylglycidate, D-8-P-Menthene-1,2-Epoxide, L-8-P-Menthene-1,2-Epoxide, 2,3-Epoxyoctanal, 2,3-Epoxyheptanal, 2,3Epoxydecanal, Hydroxy(4-Hydroxy-3-Methoxyphenyl)Acetic Acid, 4-Hydroxy-4-(3-Hydroxy-1-Butenyl)-3,5,5-Trimethyl-2-Cyclohexen-1-One, (+/−)-2,6,10,10-Tetramethyl-1-Oxaspiro[4,5]Deca-2,6-Dien-8-One, 4-(2-Butenylidene)-3,5,5-Trimethylcyclohex-2-En-1-One, Digeranyl Ether, 1-(4-Hydroxy-3-Methoxyphenyl)Decan-3-One, Alpha-Bisabolol, 2(4)-Ethyl-4(2),6-Dimethyldihydro-1,3,5-Dithiazine (Mixture Of Isomers), (2e,6e/Z,8e)-N-(2-Methylpropyl)-2,6,8-Decatrienamide, Amino-5,6-Dimethylthieno[2,3-D]Pyrimidin-2(1 h)One And 4-Amino-5,6-Dimethylthieno[2,3-D]Pyrimidin-2(1h)One Hydrochloride, 1,1-Propanedithiol, Z-5-Octenyl Acetate, (E)-4-Undecenal, Delta-Hexadecalactone, Trilobatin, L-Isoleucine, 1-(2-Furfurylthio)-Propanone, (+/−)-4-Methyl-2-Propyl-1,3-Oxathiane, N-(2-Methylcyclohexyl)-2,3,4,5,6-Pentaftuorobenzamide, Arachidonic Acid Enriched Oil, 5-Isopropyl-2,6-Diethyl-2-Methyltetrahydro-2h-Pyran, (1r,2s,5 r)-N-(4-Methoxyphenyl)-5-Methyl-2-(1-Methylethyl)Cyclohexanecarboxamide, Octahydro-4,8a-Dimethyl-4a(2h)-Naphthol, 2-Methyl-4,5-Dihydrofuran-3-Thiol, (2s,5r)-N -[4-(2-Amino-2-Oxoethyl)Phenyl]-5-Methyl-2-(Propan-2-Y1)Cyclohexanecarboxamide, (+/−)-6-Octyltetrahydro-2h-Pyran-2-One, (+/−)-2-Methyltetrahydrofuran-3-Thiol Acetate, (+/−)-3-Hydroxy-3-Methyl-2,4-Nonanedione, 1,1-Di propoxyethane, Chrysanthemum Extract, Honeysuckle Extract, Yuzunone, L-Methionylglycine, N-Cyclopropyl-5-Methyl-2-Isopropylcyclohexanecarboxamide, 3-Pentanethiol, 2-Ethyl-2,5-Dihydro-4-Methylthiazo le, 1-(Methyldithio)-2-Propanone , 5-Methylfurfurylmercaptan, 4-Mercapto-3-Methyl-2-Butanol, Ferrous L-Lactate, O-Trans-Cournaric Acid, 3-[(4-Amino-2,2-Dioxido-1h-2,1,3-Benzothiadiazin-5-Y1)Oxy]-2,2-Dimethyl-N-Propylpropanamide, 2(3),5-Dimethyl-6,7-Dihydro-5h-Cyclopentapyrazine, Cinnamyl Benzoate, Beta-Naphthyl Methyl Ether, Rosemary Oleoresin, 9-Decen -2-One, 1-(Methylthio)-3-Octanone, 3′,7-Dihydroxy4′-Methoxyflavan, Glutamyl-Valyl-Glycine, L-Threonine, Luo Han Fruit Concentrate, L-Alanyl-L-Glutamine, Sucrose Monopalmitate, Ethyl 2-Mercapto-2-Methylpropionate, 2-(3,4-Dihydroxyphenyl)-5,7-Dihydroxy-4-Chromanon, N-[N-[3-(3-Hydroxy-4-Methoxyphenyl)Propyl]-L-Alpha-Aspartyl]-L-Phenylalanine 1-Methylester, Monohydrate, Sweet Blackberry Leaves Extract, 2-[(2-(P-Menthyloxy)Ethoxy]Ethanol, Succinic Acid, Rebaudioside C, 1-(2-Hydroxyphemil)-3-(Pyridin-4-Y1)Propan-1-One, 1-(2-Hydroxy-4-Isobutoxyphenyl)-3-(Pyridin-2-Y1)Propan-1-One, 1-(2-Hydroxy-4-Methoxyphenyl)-3-(Pyridin-2-Y1)Propan-1-One, Trans-4-Tert-Butylcyclohexanol, 3-(1-((3,5-Dimethylisoxazol-4-Y1)Methyl)-1h-Pyrazol4-Y1)-1-(3-Hydroxybenzyl)Imidazolidine-2,4-Dione, 3-(1-((3,5-Dimenthylisoxazol-4-Y1)Methyl)-1h-Pyrazol-4-Y1)-1-(3-Hydroxybenzyl)-5,5-Dimethylimidazolidine-2,4-Dione, Clover Herb Distillate, Glucosyl Steviol Glycosides, D1-Isomenthol, O-Ethyl S-1-Methoxyhexan-3-Y1 Carbonothioate, Cassyrane, 1,5-Octadien-3-O1, (+/−)-2-Mercaptoheptan-4-O1, 3-(Methylthio)Decanal, (4z,7z)-Trideca-4,7-Dienal, Persicaria Odorata Oil, Amacha Leaves Extract, Glutamyl-2-Aminobutyric Acid, Glutatnyl-Norvalyl-Glycine, Glutatnyl-Norvaline, N1-(2,3-Dimethoxybenzyl)-N2-(2-(Pyridin-2-Y1)Ethyl) Oxalamide, 1-(2-Hydroxy-4-Methylcyclohexyl)Ethanone, Mexican Lime Oil, Expressed, Persian Lime Oil, Expressed, (+/−)-6-Methoxy-2,6-Dimethylheptanal, 3,5-Undecadien-2-One, (+/−)-2,5-Undecadien-1-O1, Triethylthialdine, 4-Methylpentyl 4-Methylvalerate, Cis-3-Hexenyl Salicylate, (R)-N-(1-Methoxy-4-Methylpentan-2-Y1)-3,4-Dimethylbenzamide, N-Acetyl Glutamate, 1,3-Propanediol, Szechuan Pepper Extract, Tasmannia Lanceolata Extract, Mentha Longifolia Oil, Mangosteen Distillate, Ethyl 3(2-Hydroxyphenyl)Propanoate, 1-Cyclopropanemethyl-4-Methoxybenzene, Prenyl Thioisobutyrate, Prenyl Thioisovalerate, (−)-Matairesinol, Stevioside, 1-(2,4-Dihydroxyphenyl)-3-(3-Hydroxy-4-Methoxyphenyl)Propan-1-One, Ethyl 5-Formyloxydecanoate, 3-[3-(2-Isopropyl-5-Methyl-Cyclohexyl)Ureido]Butyric Acid Ethyl Ester, 2-Isopropyl-4-Methyl-3-Thiazoline, 2,6,10-Trimethyl-9-Undecenal, 5-Mercapto-5-Methyl-3-Hexanone, Meyer Lemon Oil, Cold Pressed, Citrus X Meyeri, Steviol Glycoside Extract, Stevia Rebaudiana, Rebaudioside A 60%, Steviol Glycoside Extract, Stevia Rebaudiana, Rebaudioside A 80%, (E)-N-[2-(1,3-Benzodioxol-5-Y1)Ethyl]-3-(3,4-Dimethoxyphenyl)Prop-2-Enamide, 4-Amino-5-(3-(Isopropylamino)-2,2-Dimethyl-3-Oxopropoxy)-2-Methylquinoline-3-Carboxylic Acid, 3-Methyl-5-(2,2,3-Trimethylcyclopent-3-En-1-Y1)Pent-4-En-2-O1, (1-Methyl-2-(1,2,2-Trimethylbicyclo[3.1.0]Hex-3-Y1methyl)Cyclopropyl)Methanol, Erospicata Oil, Mentha Spicata, Curly Mint Oil, Mentha Spicata Var. Crispa, (+/−)-2-Mercapto-5-Methylheptan-4-One, Caryophylla-3(4),8-Dien-5-O1, L-Cysteine Methyl Ester Hydrochloride, 2(3)-Hexanethiol, Mixture Of 1-Vinyl-3-Cyclohexenecarbaldehyde And 4-Vinyl-1-Cyclohexenecarbaldehyde, (+/−)-4-Hydroxy-6-Methyl-2-Heptanone, 2-Octyl-2-Dodecenal., 2-Hexyl-2-Decenal, Trans-6-Octenal, (E)-3-Benzo[1,3]Dioxol-5-Y1-N,N-Diphenyl-2-Propenamide, 2,6-Dimethyl-5-Heptenol, (+/−)-Bicyclo[2.2.1]Hept-5-Ene-2-Carboxylic Acid, Ethyl Ester, 3-(Acetylthio)Hexanal, (+/−)-3-Mercapto-1-Pentanol, (3r,3s)-3-[[(4-Amino-2,2-Dioxido-1h-2,1,3-Benzothiadiazin-5-Y1)Oxy]Methyl]-N-Cyclopentyl-2-Oxo-3-Piperidinecarboxamide, (+/−))-1-Cyclohexylethanol, (+/−)-8-Methyldecanal, Steviol Glycoside Extract, Stevia Rebaudiana, Rebaudioside C 30%, (+/−)-Naringenin, 2-(((3-(2,3-Dimethoxyphenyl)-1h-1,2,4-Triazol-5-Y1)Thio)Methyl)Pyridine, (2r)-3′,5-Dihydroxy-4′-Methoxyflavanone, Glucosylated Rubus Suavissimus Extract, 20-30% Glucosylated Rubusoside Glycosides, Olive Fruit Extract, (S)-1-(3-(((4-Amino-2,2-Dioxido-1h-Benzo[C][1,2,6]Thiadiazin-5-Y1)Oxy)Methyl)Piperidin-1-Y1)-3-Methylbutan-1-One, 8-Methylnonanal, Mixture Of Ricinoleic Acid, Linoleic Acid, And Oleic Acid, Steviol Glycoside Extract, Stevia Rebaudiana, Rebaudioside A 22%, Steviol Glycoside Extract, Stevia Rebaudiana, Rebaudioside C 22%, Pinocarvyl Acetate, N-Ethyl-5-Methyl-2-(1-Methylethenyl)Cyclohexanecarboxamide, 2-(4-Methylphenoxy)-N-(1h-Pyrazol-3-Y1)-N-(Thiophen-2-Y1methyl)Acetamide, Ethyl-2-(4-Hydroxy-3-Methoxy-Phenyl)Acetate, Ginger Mint Oil (Mentha×Gracilis), Pahnitoylated Green Tea Extract Catechins, 2-(5-Isopropyl-2-Methyltetrahydrothiophen-2-Y1)Ethanol, Glucosylated Rubus Suavissimus Extract, 60% Glucosylated Rubusoside Glycosides, Sandalwood Austrocaledonicum Oil, and Sugar Cane Distillate.

In some embodiments, the combination of the umami flavor compound or umami flavor enhancing compounds with the flavorants described above has a synergistic effect. For example, the combination may further enhance the umami flavor of the composition or enhance the flavor properties of the flavorant.

### Sweeteners

In some embodiments, the composition comprises an umami flavor compound or umami flavor enhancing compound and one or more sweeteners, sweet flavorants, or sweet taste enhancers. Representative sweeteners, sweet flavorants, or sweet taste enhancers include but are not limited to: natural or synthetic carbohydrates or carbohydrate analogues, monosaccharides, disaccharides, oligosaccharides, and polysaccharides, rare sugars, sugars in either of the D- or L-conformations, sucrose, fructose, glucose, L-arabinose, L-fucose, L-glucose, L-ribose, D-arabino-hexulose, psicose, altrose, arabinose, turanose, abequose, allose, abrusoside A, aldotriose, threose, xylose, xylulose, xylo-oligosaccharide (such as xylotriose and xylobiose), lyxose, polydextrose, oligofructose, fucose, galacto-oligosaccharide, galactosamine, galactose, gentio-oligosaccharide (such as gentiobiose, gentiotriose, and gentiotetraose), dextrose, cellobiose, D-leucrose, D-psicose, D-ribose, D-tagatose, trehalose (mycose), neotrehalose, isotrehalose, raffinose, idose, tagatose, melibiose, mannan-oligosaccharide, rhamnose, ribose, ribulose, malto-oligosaccharide (such as maltotriose, maltotetraose, maltopentaose, maltohexaose, and maltoheptaose), maltose, sucrose acetate isobutyrate, dextrose, erythrose, erythrulose, deoxyribose, gulose, ketotriose, lactose, lactulose, kestose, nystose, mannose, sucralose, palatinose, polydextrose, sorbose, sugaridextrose (blended sugar), talose, sweetener compositions comprising one or more natural or synthetic carbohydrate, corn syrup, high fructose corn syrup, high maltose corn syrup, glucose syrup, sucralose syrup, hydrogenated glucose syrup (HGS), hydrogenated starch hydrolyzate (HSH), other syrups or sweetener concentrates derived from natural fruit and vegetable sources, semi-synthetic “sugar alcohol” sweeteners, polyols, erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, sugar alcohols, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatfolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside 1, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, periliartine, petphyllum, phenylalanine, phlomisoside I, phlorodizin, phynodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, chemically or enzymatically modified natural high potency sweeteners, glycosylated natural high potency sweeteners, glucosyl-derivatives containing 1-50 glycosidic residues, galactosyl-derivatives containing 1-50 glycosidic residues, fructosyl-derivatives containing 1-50 glycosidic residues, Theasaponin E1, Acesulfame K Alitame, Aspartame, CH 401, Dulcin, Erythritol, Guanidine Sweetener, Isoralt, Isomaltosylfructoside, Isoraffinose, NC 174, Neotame, Perillartine, Phenylacetylglycyl-L-Lysine, Saccharin, SC 45647, sodium Cyclamate, Sorbitol, Sucralose, Sucrononic Acid, Suosan, Superaspartame, Methyl alpha-L-arabinoside, Methyl beta-L-arabinoside, Methyl beta-D-Glucoside, Methyl a-D-mannoside, Methyl beta-L-xylopyranoside, Methyl alpha-D-xyloside, Methyl alpha-D-Glucoside 2,3-Di-threonine, Methyl alpha-D-Glucoside 2,3-Di-isoleucine, Protocatechuic Acid, Cynarin, Glycyphyllin, Rebaudioside C, Abrusoside A, Abrusoside B, Abrusoside C, Abrusoside D, Abrusoside E, Apioglycyrrhizin, Araboglycyrrhizin, Baiyunoside, Brazzein, Bryodulcoside, Carnosifloside V, Carnosifloside VI, D. cumminsii, Cyclocarioside A, Cyclocarioside I, Dulcoside A, Fluorene-4-alpha,6-dicarboxylic acid, 4-beta,10-alpha-dimethyl-1,2,3,4,5,10-hexahydor-Gaudichaudioside A, Glycyrrhizic Acid, Hernandulcin, Hernandulcin, 4beta-hydroxy-Hesperitin-7-Glucoside Dihydrochalcone, Huangqioside E, Huangqioside E, 3-Hydroxyphloridzin, Kaempferol, 2,3-Dihydro-6-Methoxy 3-O-Acetate, Mabinlin Maltosyl-Alpha-(1,6)-Neohesperidin Dihydrochalcone, Mogroside IIE, Mogroside III, Mogroside IIIE, Mogroside IV, Mogroside V, 11-Oxo Mogroside V, Monatin, Monellin, Monoammonium Glycyrrhizinate (Mag), Mukurozioside lib, Naringin Dihydrochalcone, Neoastilbin, Neohesperidin Dihydrochalcone (NHDHC), Neomogroside, Osladin, Pentadin, Periandrin I, Periandrin II, Periandrin lll, Periandrin IV, Periandrin V, Phlomisoside I, Phlorizin, Phyllodulcin, Polypodoside A, Potassium magnesium calcium glycyrrhizin, Pterocaryosides A, Pterocaryosides B, Quercetin, 2,3-Dihydro-3-O-Acetate, Quercetin, 2,3-Dihydro-6-Methoxy-Quercetin, 2,3-Dihydro-6-Methoxy-3-O-Acetate, Rebaudioside A, Rebaudioside B, Rubusoside, Scandenoside R6, Siamenoside I, Sodium glycyrrhizinate, Steviolbioside, Stevioside, Stevioside, alpha-Glycosyl Suavioside A, Suavioside B, Suavioside G, Suavioside H, Suavioside I, Suavioside J, Thaumatin, Triammonium Glycyrrhizinate (TAG), Trilobatin Selligueain A, Haematoxylin, Maltitol, Mannitol, Methyl alpha-D-Glucoside 2,3-Di-aspartic acid, Benzoic Acid, 2-(4-Dimethylaminobenzoyl)-Benzoic Acid, 2-Hydroxy-4-aminomethyl-Benzoic Acid, 2-(3-Hydroxy-4-Methoxybenzoyl)-Methyl beta-D-fructoside, Methyl alpha-D-galactoside, Methyl beta-D-galactoside, Cureulin, Strogin 1, Strogin 2, Strogin 4, Miraculin, Phenylacetic Acid, 3,4-Dimethoxy-Aminobenzoic Acid, 3-Anisic Acid, Benzyl alcoho, 3-Amino-4-n-propoxyl, 3,4-Caffeic Acid, Cinnamic Acid, Dihydroxycinnamic Acid, 2,4-Ferulic Acid, Hydrolyzed Guar Gum, Hydroxyaminobenzoic Acid, 2,4-Nigerooligosaccharides, Sugarcane Bagasse Extract, Dihydroxybenzoic Acid, 2,3-Dihydroxybenzoic Acid, 2,4-Coumaric Acid, p-Dihydroxybenzoic Acid, 3,5-Hydroxybenzoic Acid, 3-Gurmarin, Gymnemasaponin III, Gymnemasaponin IV, Gymnemasaponin V, Gymnemic Acid I, Gymnemic Acid II, Gymnemic Acid III, Gymnemic Acid IV, Hodulcin, Jujubasaponin II, Jujubasaponin III, Propionic Acid, (−)-2-(4-Methoxyphenoxy)-Ziziphin, Ethyl Maltol, Maltol, Butanoic Acid, 2-Oxo-3-Methyl-Alanine, N-(1-Methyl-4-oxo-2-imidazolin-2-yl) Creatinine, Abrusoside E, mono-methyl ester, Lactitol, Periandrinic acid I, monoglucuronide, Periandrinic acid II, monoglycuronide, Xylitol, Tagatose, d-Benzoyloxyacetic acid, 4-Methoxy Hoduloside I, 4-Nitrophenyl a-D-galactoside, 4-Nitrophenyl alpha-D-glucoside, 4-Nitrophenyl beta-D-glucoside, 4-Nitrophenyl alpha-D-mannopyranoside, Urea, (N-(4-cyanophenyl)-N′-((sodiosulfo)methyl)-Chloramphenicol, Chlorogenic Acid, Methyl alpha-D-Glucoside, Methyl alpha-D-Glucoside 2,3-Di-alanine, Methyl alpha-D-Glucoside 2,3-Di-glycine, Methyl alpha-D-Glucoside 2,3-Di-proline, Methyl alpha-D-Glucoside 2,3-Di-valine, Aniline, 2-Butoxy-5-Nitro-Aniline, 2-Ethoxy-5-Nitro-Aniline, 2-Methoxy-5-Nitro-Aniline, 3-Nitro-(+)-Baiyunol-beta-D-g glucoside-alpha-D-glucoside, Aniline, 1,3-Hydroxy-4-methoxybenzylAniline, 2-Propxy-5-Nitro-(P4000)Benzo-1,4-dioxane 2-(3-Hydroxy-4-Methoxyphenyl)-Benzoe-1,3-dioxan-4-one 2-(3-Hydroxy-4-methoxyphenyl)-Benzoic Acid, 2-Benzoyl-4-Methoxy-Benzoic Acid, 2-(4-Methoxybenzoyl)-Benzo-1,3(4H)-xathiane, 2-(3-Hydroxy-4-Methoxyphenyl)-Benzo-1,4-xathiane 3-(3-Hydroxy-4-methoxyphenyl)-Butanoic acid, 4-[3,5-dihydroxy-4[3-(3-hydroxy-4-methoxyphenyl)-1-oxopropyl]phenoxy]-2-h-ydroxy-monosodium salt, Butanoic acid, 4-[3,5-dihydroxy-4-[3-(3-hydroxy-4-methoxyphenyl)-1-oxopropyl]phenoxy]-3-oxo-monosodium salt, Cyclohexadiene-1,4 1-Carboxaldehyde-4-(Methoxymethyl)-, (E)oxime Ethylbenzene, beta-(1,3-Hydroxy-4-methoxybenzyl)-Hespertin Dihydrochalcone, 3′-Carboxy-Hespertin Dihydrochalcone, 3′-Formyl-Isocoumarin, 3,4-Dihydro-3-(3-Hydroxy-4-methoxy) Perillartine, 8,9-epoxy-Phenyl 3-Hydroxy-4-methoxybenzyl Ether, Phosphonic acid, [3-[3,5-dihydroxy-4-[3-(3-hydroxy-4-methoxyphenyl)-1-oxopropyl]phenoxy]propyl] monopotassium salt, Stevioside analogue, Sulfamic acid, [2-[3,5-dihydroxy-4-[3-(3-hydroxy-4-methoxyphenyl)-1-oxopropyl]phenoxy] ethyl]-monopotassium salt, Urea, and N-(4-cyanophenyl)-N′-(2-carboxyethyl)-L-Theanine.

In some embodiments, the combination of the umani flavor compound or umami flavor enhancing compounds with the sweeteners described above has a synergistic effect. For example, the combination may further enhance the umami flavor of the composition or enhance the flavor or other properties of the sweetener.

### Bitterants

In some embodiments, the composition comprises an umami flavor compound or umami flavor enhancing compound and one or more bitterants, bitter flavor compounds, or bitterness-enhancing compounds. Representative bitterants, bitter flavor compounds, and bitterness-enhancing compounds include but are not limited to: Acteoside, Adhumulone, Adlupulone, Aesculetin, Aesculin, L-Alanine, L-alanyl-L-alanyl-L-Alanine, L-alanyl-L-isoleucyl-Alanine L-, L-valyl-L-valyl-Amarogentin, Amaropanin Amaroswerin, Amygdalin, Angustifoline, Antiacetylhumulone, Antiisohumulone, Arginine, L-Arginyl Leucine, Arginyl Leucy Leucine, Arginyl Proline, Asaronaldehyde, Aspartyl Aspartic acid, Asparasaponin I, Atropine, Benzyl beta-D-arabinoside, Benzyl beta-L-arabinoside, Benzyl beta-D-fructoside, Benzyl beta-D-galactoside, Benzyl alpha-D-glucoside, Benzyl beta-D-glucoside, Benzyl alpha-D-mannoside, Bitter Peptides, Bitter Peptides from Soy Proteins, Butyl alpha-D-glucoside, Butyl beta-D-glucoside, Caffeine, Carnosifloside II, Carnosifloside III, Carnosifloside IV, cascara sagrada solid extract Catechin, Epicatechin, Epicatechin gallate, Chaconine, alpha-Chaconine, beta2-Chloramphenicol, Cholic Acid, Cichoriin, Cohumulone, Colupulone, Cryptochlorogenic Acid, gamma-lactone, Cucurbitacin B, Cucurbitacin D, Cyclo Alanine-glycine, Cyclo Alanine-phenylanaline, Cyclo Alanine-valine, Cyclo(L-arginylglycyl-L-prolyl-L-prolyl-L-phenylalanyl-L-isoleticyl-L-valyl), Cyclo Asparagine-phenylalanine, Cyclo Glycine-phenylalanine, Cycloheximide Cyclo Lucine-Tryptophan, Cyclopent(b)azepin-8(1H)-one, 7-Methyl-2,3,6,7-Tetrahydro-Cyclopent(b)azepin-8(1H)-one, 2,3,6,7-tetrahydro-7-hydroxy-7-methyl-Cyclopent-2-en-1-one, 2,5-dihydroxy-5-methyl-3-(1-piperidinyl)-Cyclopent-2-en-1-one, 2,5-dihydroxy-5methyl-3-(1-pyrrolidinyl) Cyclopent-2-en-1-one, 2,3-di-1-pyrrolidinyl-Cyclopent-2-en-1-one, 5-hydroxy-5-methyl-2,3-di-1-piperidinyl-Cyclopent-2-en-1-one, 5-hydroxy-5-methyl-2,3-di-1-pyrrolidinyl-Cyclopent-2-en-1-one, 5-methyl-2,3-di-1-pyrrolidinyl-Cyclopent-2-en-1-one, 5-methylene-2,3-di -1-pyrrolidinyl-Cyclopent-2-en-1-one, 3-methyl-2-(1-pyrrolidinyl)-Cyclo Phenyalanine-aspartic acid, Cyclo Proline-alanine, Cyclo Proline-asparagine, Cyclo Proline-glycine, Cyclo Proline-isolucine, Cyclo Proline-leucine, Cyclo Proline-methionine, Cyclo Proline-phenylalanine, Cyclo Proline-proline, Cyclo Proline-valine, Cyclo Valine-phenylalanine, Cynaratriol, Cynaropicrin, Cynaropicrin, Daidzein, Daidzin Denatonium benzoate, Denatonium saccharide, Dhurrin, Dihydroxybenzoic Acid, 2,3-Dihydroxybenzoic Acid, 2,4-Ethyl b-L-arabinoside, Ethyl alpha-D-Glucoside, Ethyl beta-D-Glucoside, Eustomoroside, Eustomoside, Gallic Acid, Epigallocatechin, Epigaliocatechin gallate, Gaudichaudioside F, Gelidoside, Genistein, Genistin, Gentiopicroside, Gentistic Acid, Gentomoside, Geshoidin, 6′-O-beta-D-Glucosylgentiopicroside, ucozaluzanin C, Glutamyl Aspartic Acid, Glutamyl Glutamic Acid, Glycyl Leucine, Goitrin, Gramine, Grosshemin, Hacmatoxylin Tetramethyl Ether Helicin, Heptadeca-16-ene, 1-Acetoxy-2,4-Dihydroxy-Heptadeca-16-ene, 1,2,4-Trihydroxy-Histidine, L-Hulupone, Humulinone, Humulone, Hydroxybenzoic Acid, 4-Hymenoside A, Hymenoside B, Hymenoside C, Hymenoside D, Hymenoside E, Hymenoside F, Isohumulone, cis-Isohumulone, trans-Isoleucine, L-Isolupanine, Isosparteine, beta-Isosparteine, 10,17-Dioxo-beta-Isosparteine, 10-oxo-beta-Lactucin, L-Leucine, L-alanyl-L-alanyl-L-Leucine,N-[(R2R) -6-arnino-2-[(4S)-2,5-dioxo-4-(phenylmethyl)-1-imidazolidinyl]-1-oxohexyl]-L-leucyl-L-methionyl-N-methyl-L-phenylalanyl-, (4-1)-lactam, L-Leucine, glycyl-L-alanyl-Leucine, L-L-Leucine, N-(N2-L-leucyl-L-glytaminyl)-L-Leucine, N-(N-L-leucyl-L-a-glutamyl)-L-Leucine, N-[N2-[N2-[N-(1-L-leucyl-L-prolyl)-L-phenylalanyl]-L-asparaginyl]-L-gluta-minyl]-L-Leucine, N[N2-[N-[N-(1-L-leucyl-L-prolyl)-L-phenylalanyl]-L-seryl]-L-glutaminyl]-L-Leucine, L-leucyl-L-valyl-Leucy Leucine, Leucyl Phenylalanine, Limonin, Limoninmonolactone, Linamarin, Lotaustratin, Lupine, Lupanine, 13-Hydroxy-Lupanine, 7-hydroxy-Lupinine, Epilupinine Lupoxes B, Lupoxes C, Lupulone, Luputrione, Mellein, 6-Methoxy-Methionine, L-Methyl alpha-L-arabinoside, Methyl beta-L-arabinoside, Methyl beta-D-Glucoside, Methyl alpha-D-Glucoside 2,3-Di-isoleuceine, Methyl alpha-D-Glucoside 2,3-Di-leucine, Methyl alpha-D-Glucoside 2,3-Di-L-phenylalanine, Methyl alpha-D-Glucoside 2,3-Di-threonine, Methyl alpha-D-Glucoside 2,3-Di-tyrosine, Methyl a-D-mannoside, Methyl beta-L-xylopyranoside, Methyl alpha-D-xyloside, Naringin, Neochlorogenic Acid, gamma-Lactone, Neohesperidin dihydrochalcone, Nuezhenide, Oleonuezhenide, Oleuropein, Olivieroside A, Olivieroside B, Olivieroside C, Perrottetin H, Phenylalanine, L-Phenyl alpha-D-galactoside, Phenyl alpha-D-glucoside, Phenyl beta-D-glucoside, Phenylthiourea. Phlomisoside II, Piperidine-2-carboxylic acid, 4-[(2-carboxy-2-hydroxyethyl(thio]-Piperidinecarboxylic acid-2, 4-[(2-carboxy-2-hydroxyethyl)thio]-Prehumulone, Prelupulone, Propyl beta-D-fructoside, Propyl alpha-D-glucoside, Propyl beta-D-glucoside, Protocatechuic Acid, Prunasin, Pulcherrimine, Quinidine, Quinine, Quinolizinium-7-olate, Ranitidine, Rebaudioside C, Salicin, Salidroside, Scabraside, Scandenoside R5, Sclareolide, Scopolin, Septemfidoside, Seryl Lysyl Glycyl Leucine, Sinapine, Solanine, alpha-Sparteine, Sparteine, 17-oxo-Stevisalioside A, Strychnine, Suavioside C1, Suavioside D2, Suavioside F, Sucrose Octaacetate, Sweroside, Swertiamarin, Swertiapunimarin, Taxiphyllin, TFI (Furostan, beta-D-galactomanoside), Theaflavin, Theaflavin Gallate A, Theaflavin Gallate B, Tomatidine, Tomatine, alpha-Tricyclodehydroisohumulone, Trifloroside, Trihydroxyhenzoic Acid, 2,4,6-Tryptophan, L-Uracil, 6-propyl-2-thio-L-Valine, L-arginylglycyl-L-prolyl-L-prolyl-L-phenylalanyl-L-isoleucyl-(BPIa)Valine, L-Yohimbine, extract of wild cucumber, Denatonium, denatonium benzoate, and denatonium saccharide.

In some embodiments, the combination of the umami flavor compound or umami flavor enhancing compounds with the bitterants described above has a synergistic effect. For example, the combination may further enhance the umami flavor of the composition or enhance the properties of the bitterant.

### Sour Flavorants

In some embodiments, the composition comprises an umami flavor compound or umami flavor enhancing compound and one or more acids or sour flavorants. Representative sour flavorants include but are not limited to: ascorbic acid, benzoic acid, gallic acid, glucuronic acid, adipic acid, glutaric acid, malonic acid, succinic acid, malic acid, acetic acid, lactic acid, citric acid, tartaric acid, fumaraic acid, phosphoric acid, pyrophosphoric acid, tannic acid, vinegar, lemon juice, lime juice, acidic fruit juices, and acidic fruit extracts.

In some embodiments, the combination of the umami flavor compound or umami flavor enhancing compounds with the sour flavorants described above has a synergistic effect. For example, the combination may further enhance the umami flavor of the composition or enhance the properties of the sour flavorant.

### Salty Flavorants

In some embodiments, the composition comprises an umami flavor compound or umami flavor enhancing compound and one or more salts or salt flavor enhancers. Representative salts or salt flavor enhancers include but are not limited to: mineral salts, sodium chloride, potassium chloride, magnesium chloride, ammonium chloride, sodium gluconate, sodium phosphates, glycine, L-alanine, L-valine, L-leucine, L-isoleucine, L-phenylalanine, L, tyrosine, L-glutamine, L-glutamic acid, L-asparagine, L-aspartic acid, L-serine, L-threonine, L-cysteine, L-methionine, L-proline, L-Lysine, L-arginine, L-tryptophan, L-histidine, L-pyrolysine, L-pyroglutamine, L4-trans-hydroxyproline, L-3-cis-hydroxyproline, L-homoserine, L-homocysteine, L-cystine, L-ornithine and L-citrulline, L-glutamine, L-glutamic acid, L-asparagine, L-aspartic acid, L-valine, L-arginine and L-lysine.

In some embodiments, the combination of the umami flavor compound or umami flavor enhancing compounds with the salty flavorant described above has a synergistic effect. For example, the combination may further enhance the umami flavor of the composition or enhance the properties of the salty flavorant. In some embodiments, the combination imparts a synergistic salt flavor.

### Combination of Umami Flavorants

In some embodiments, the composition comprises an umami flavor compound or umami flavor enhancing compound and one or more additional umami flavor compounds or umami flavor enhancing compounds. Representative umami flavor compounds or umami flavor enhancing compounds include but are not limited to the compounds identified in U.S. Patent Application Publications 2005/0084506A1, U.S. 2009/0111834A1, U.S. 2012/0201763A1, U.S. 2015/0093339A1, U.S. 2006/0263411A1, U.S. 2012/0226047A1,and 2009/0220662A1. Additional representative umami flavor compounds or umami flavor enhancing compounds include but are not limited to: meats and meat products including cured meats and extracts thereof, hydrolyzed soy protein, hydrolyzed corn protein, wheat and wheat gluten, hydrolyzed wheat protein, fresh and dried seafood, fish and fish products and extracts thereof, including tuna, mackerel, anchovy, anchovy paste, Bonito flakes, fish sauce, shellfish, including shrimp and prawns, shrimp powder, fish powder, shrimp sauce, yeast extract, including Marmite™ and Vegemite™, nutritional yeast, hydrolyzed yeast extract, autolyzed yeast extract mushrooms, mushroom powder, dehydrated mushrooms, mushroom extract, kombucha, hydrolyzed vegetable protein, oyster sauce, soy sauce, soy extract, tamari, miso powder, miso paste, dashi, cheeses including aged cheeses, parmesan cheese, parmesan cheese solids, kombu powder, kombu, dehydrated kombu, kombu paste, nori, nori powder, nori paste, seaweed, dehydrated seaweed, seaweed powder, seaweed extract, tomato, dehydrated tomato, tomato powder, tomato sauce, tomato paste, tomato extract, ketchup, banana sauce, Worcestershire sauce, vegetables, including cabbage, Chinese cabbage, spinach, celery, potatoes, sweet potatoes, asparagus, soybeans, vegetable powder, artichoke, artichoke powder, cabbage powder, Chinese cabbage powder, spinach powder, celery powder, potatoes powder, sweet potato powder, asparagus powder, soybean powder, vegetable extract, artichoke extract, cabbage extract, Chinese cabbage extract, spinach extract, celery extract, potatoes extract, sweet potato extract, asparagus extract, soybean extract, green tea, green tea extract, green tea powder, whey powder, whey solids, whey, collagen, gelatin, textured vegetable protein, sodium caseinate, calcium caseinate, magnesium caseinate, potassium caseinate, glyoxylic acid, 3-methyl-2-oxo-butanoic acid, 3-methyl-2-oxo-pentanoic acid, 4-methyl-2-oxo-pentanoic acid, 3-hydroxy-2-oxo-propanoic acid, oxalacetic acid, 2-oxo-glutaric acid, 2-oxo-3-phenyl-propanoic acid, 3-(4-hydroxyphenyl)-2-oxo-propanoi c acid, 2-oxo-1H-indole-3-propanoic acid, 2-oxo-1H-imidazole-4-propanoic acid, 4-methylthio-2-oxo-butanoic acid, 3-mercapto-2-oxo-propanoic acid, 3-hydroxy-2-oxo-butanoic acid, 6-amino-2-oxo-hexanoic and 5-guanidino-2-oxo-pentanoic acid, 2-amino-butanoic acid, alpha.-alanine, glycine, norvaline, valine, aspartic acid, norleucine, leucine, isoleucine, serine, threonine, glutamic acid, phenylalanine, tyrosine, cysteine, methionine, lysine, tryptophane, histidine, arginine, asparagine, glutamine, cystine, citrulline theanine, .gamma.-methylene-glutamic acid, isoeugcmol, 2-propylphenol, p-vinylguaiacol, 2-acetylpyrazine, 2-ethyl-3,5-dimethylpyrazine, 2,3,5-trimethylpyrazine, 2,3-diethyl-5-methylpyrazine, 3-ethyl-2-methylpyrazine, dimethyl sulfide, dimethyl disulfide, dimethyl trisulfide, methylpropyl disulfide, 2-methylthiophenol, methional (3-methylthiopropanal), 2-octenal, 2,4-nonadienal, 2,4-decadienal, 2,4-undecadienal, 2-methoxybenzaldehyde, 2,4-dodecadienal, decenal, methyl 2-furanecarboxylate, 2-ethyl-4-hydroxy-3-methyl 5(2H)-furanone, 2,6-dimethylbenzenethiol 2-nonen-1-ol, 10-undecenoic acid, undecanoic acid, isodecanoic acid and isononanoic acid, 2-oxo-butanoic acid, oxalacetic acid, 3-methyl-2-oxo-butanoic acid, 3-methyl-2-oxo-pentanoic acid, 2-oxo-glutaric and 3-mercapto-2-oxo-propanoic acid, NaCl, KCl, MSG, guanosine monophosphate (GMP), inosin monophosphate (IMP), ribonucleotides such as disodium inosinate, disodium guanylate, N-(2-hydroxyethyl)-lactamide, N-lactoyl-GMP, N-lactoyl tyramine, gamma amino butyric acid, allyl cysteine, 1-(2-hydroxy-4-methoxylphenyl)-3-(pyridine-2-yl)propan-1-one, arginine, potassium chloride, ammonium chloride, succinic acid, N-(2-methoxy-4-methyl benzyl)-N′-(2-(pyridin-2-yl)ethyl) oxalamide, N-(heptan-4-yl)benzo(D)(1,3)dioxole-5-carboxamide, N-(2,4-dimethoxybenzyl)-N′-(2-(pyridin-2-yl)ethyl) oxalamide, N-(2-methoxy-4-methyl benzyl)-N′-2(2-(5-methyl pyridin-2-yl)ethyl) oxalamide, cyclopropyl-E,Z-2,6-nonadienamide, glutamic acid, glutamate, monosodium glutamate, monopotassium glutamate, monoammonium glutamate, calcium diglutamate, magnesium diglutamate, L-asparagine or a salt thereof, 5′-ribonucleotides or their salts, calcium 5′-ribonucleotides, disodium 5′-ribonucleotides, dipotassium 5′-ribonucleotides, inosinic acid, guanylic acid, adenosinic acid, inosinates, guanylates, and adenylates, guanosine 5′-monophosphate, inosine 5′-monophosphate, 5′-adenylate, disodium guanylate, disodium inosinate, disodium adenylate; dipotassium guanylate, dipotassium inosinate, dipotassium adenylate, calcium guanylate, calcium inosinate, calcium adenylate, maltol, ethyl maltol, glycine, L-leucine, autolyzed or hydrolyzed proteins (e.g. autolyzed yeast, hydrolyzed yeast, hydrolyzed vegetable proteins), Koji-Aji (Ajinomoto Food Ingredients), fermented wheat gluten, Glutathione, Glutamyl Glutamic Acid, (Z)-6-Dodeeen-4-olide, Inosinic acid, Dodee-Z6-en-4-olide, Glutamic Acid, L-Aconitic Acid, N-(1-deoxy-fructos-1-yl) glutamate, hydrolyzed vegetable protein, Methyl alpha-D-Glucoside, 2,3-Di-lysine, Methyl alpha-D-Glucoside 2,3-Di-ornithine, L-Asparagine, L-a-glutamyl-L-a-glutamyl-L-Glutamic acid, L-a-aspartyl-L-a-glutamyl-Glutamyl valine, Wheat gluten hydrolyzate, Aspartic acid L-, L-a-aspartyl-L-a-aspartyl-L-a-aspartyl-Docosahexaenoic acid, and (4Z,7Z,10Z,13Z,16Z,19Z)-L-Theanine, allyl cysteine, propenyl cysteine, S-(.alpha.,.beta.-dicarboxyethyl) .gamma.-L-glutamyl-L-cysteinyl-glycine, S-(.alpha., .beta.-dicarboxyethyl) cysteine, 3-(carboxymethoxy)-alanine, S-carboxymethyl-glutathione (glutaramic acid), S-carboxymethyl-cysteinyl-glycine, (S-carboxymethyl)-lysyl-cysteine, S-dicarboxymethyl-glutathione, S-carboxytnethyl-cysteine, S-(1,2-dicarboxyethyl)-glutathione, and S-(1,2-dicarboxyethyl)-cysteine, N-acetyl GMP, N-formyl GMP, N-propanoyl GMP, N-butanoyl GMP, N-pentanoyl GMP, N-hexanoyl GMP, N-heptanoyl GMP, N-octanoyl GMP, N-oxalyl GMP, N-succinyl GMP, N-glutaryl GMP, N-fumaryl GMP, N-maleyl GMP, N-adipyl GMP, N-citryl GMP N-galloyl GMP, N-oxalacetyl-GMP, N-feruloyl GMP, N-pyruvyl GMP, N-benzoyl GMP, N-vanilloyl GMP, N-anthranoyl GMP, N-caffeoyl GMP, N-cinnamoyl GMP, N-acetyl AMP, N-formyl AMP, N-propanoyl AMP, N-butanoyl AMP, N-pentanoyl AMP, N-hexanoyl AMP, N-heptanoyl AMP, N-octanoyl AMP, N-oxalyl AMP, N-succinyl AMP, N-glutaryl AMP, N-fumaryl AMP, N-maleyl AMP, N-adipyl AMP, N-citryl AMP, N-galloyl AMP, N-oxatacetyl-AMP, N-feruloyl AMP, N-pyruvyl AMP, N-benzoyl AMP, N-vanilloyl AMP, N-anthranoyl AMP, N-caffeoyl AMP, N-cinnamoyl AMP, N-acetyl CMP, N-formyl CMP, N-propanoyl CMP, N-butanoyl CMP, N-pentanoyl CMP, N-hexanoyl CMP, N-heptanoyl CMP, N-octanoyl CMP, N-oxalyl CMP, N-succinyl CMP, N-glutaryl CMP, N-fumaryl CMP, N-maleyl CMP, N-adipyl CMP, N-citryl CMP, N-galloyl CMP, N-oxatacetyl-CMP, N-feruloyl CMP, N-pyruvyl CMP, N-benzoyl CMP, N-vanilloyl CMP, N-anthranoyl CMP, N-caffeoyl CMP, N-cinnamoyl CMP, N-acetyl GMP, N-formyl GMP, N-propanoyl GMP, N-butanoyl GMP, N-pentanoyl GMP, N-hexanoyl GMP, N-heptanoyl GMP, N-octanoyl GMP, N-oxalyl GMP, N-succinyl GMP, N-glutaryl GMP, N-fumaryl GMP, N-maleyl GMP, N-adipyl GMP, N-citryl GMP, N-galloyl GMP, N-oxatacetyl-GMP, N-feruloyl GMP, N-pyruvyl GMP, N-benzoyl GMP, N-vanilloyl GMP, N-anthranoyl GMP, N-caffeoyl GMP, N-cinnamoyi GMP, flavor modifiers created by maillard reactions, S-(.alpha.,.beta.-dicarboxyethyl) .gamma.-L-glutamyl-L-cysteinyl-glycine, S-(.alpha., .beta.-dicarboxyethyl) cysteine, 3-(carboxymethoxy)-alanine, S-carboxymethyl-glutathione (glutaramic acid), S-carboxymethyl-cysteinyl-glycine, (S-carboxymethyl)-lysyl-cysteine, S-dicarboxymethyl-glutathione, S-carboxymethyl-cysteine, S-(1,2-dicarboxyethyl)-glutathione, and S-(1,2-dicarboxyethyl)-cysteine, Gamma-L-glutamyl-L-cysteinyl-glycine or y-Glu-Cys-Gly, Rubemamine, rubemamide, rubescenamine, Rubescenamide, zanthosine, zanthosinamide, dioxamide, dioxamine, zanthomamine, and zanthomamide.

Umami flavor compounds or umami flavor enhancing compounds may also include peptides with the sequence Lys-Ile-His-Pro-Phe, Gly-Pro-Phe-Pro-Ile, or Lys-Lys-Tyr-Lys-Val-Pro-Gln, Glu-Glu-Leu, Glu(Glu-Leu), Glu-Asp-Phe, Glu-Glu-Ile, Asp-Glu-Leu and Glu-Leu-Glu. And their N-lactoyl derivatives, Lys-Gly Asp.-Glu-Glu-Ser-Leu-Ala, Ser-Leu-Ala-Lys-Gly-Asp-Glu-Glu, Ser-Leu-Ala-Asp-Glu-Glu-Lys-Gly, Lys-Gly-Ser-Leu-Ala,-Asp-Glu-Glu, Lys-Gly-Asp-Glu-Glu, Glu-Glu-Asp-Gly-Lys, or Asp-Glu-Glu (See Nakata et al., Biosci. Biotechnol. Biochem. (1995) 59(4):689-93).

In some embodiments, the combination of multiple umami flavor compound or umami flavor enhancing compounds described above has a synergistic effect. For example, the combination may further enhance the umami flavor of the composition beyond a merely additive effect.

### Plant or Animal Products

In some embodiments, the composition comprises an umami flavor compound or umami flavor enhancing compound and one or more plant or animal products. Representative plant or animal products include but are not limited to: zedoary bark extract (curcuma zedoaria (berg.) rosc.), zedoary (curcuma zedoaria (berg.) rosc.), alfalfa extract (medicago sativa l.), alfalfa herb and seed (medicago sativa l.), algae brown extract (macrocystis and laminaria spp.), algae red (porphyra spp. and gloiopeltis furcata and rhodymenia palmata (l.)), algae red extract (porphyra spp. and gloiopeltis furcata and rhodymenia, alkanet root extract (alkanna tinctoria tausch), allspice (pimenta officinalis lindl.), allspice, oil (pimenta officinalis lindl.), allspice, oleoresin (pimenta officinalis lindl.), almond, bitter, oil (ffpa) (prunus spp.), aloe, extract (aloe spp.), ambrette, absolute, oil (hibiscus abelmoschus l.), ambrette seed (hibiscus abelmoschus l.), ambrette seed, oil (hibiscus abelmoschus l.), ambrette, tincture (hibiscus abelmoschus l.) amyris (amyris balsamifera l.), amyris, oil (amyris balsamifera l.) angelica root (angelica spp), angelica root, extract (angelica archangelica l.), angelica root, oil (angelica archangelica l.), angelica seed (angelica spp.), angelica seed, extract (angelica archangelica l.), angelica seed, oil (angelica archangelica L), angelica stem, oil (angelica archangelica l.), angola weed (roccella fuciformis ach.), angostura (galipea offincinalis hancock), angostura, extract (galipea officinalis hancock), anisaldehyde propyleneglycol acetal, anise (pimpinella anisum l.), anise, oil (pimpinella anisum l.), anise, star (illicium verum hook, f.), anise, star, oil (illicium verum hook, f.), annatto, extract (bixa orellana l.) annatto, seed (bixa orellana l.), apple essence, natural, apricot kernel, oil (prunus armeniaca l.), arnica flowers (arnica spp.), arrowroot starch, artemisia (artemisia spp.), artemisia extract, artemisia oil, artichoke leaves (cynara scolymus l.), asafetida., fluid extract (ferula assafoetida l.), asafetida, gum (ferula assafoetida l.), asafetida, oil (ferula assafoetida l.), asparagus, seed and root, extract, bakers yeast extract, baker's yeast glycan, baker's yeast protein, balm (melissa officinalis l.), balm leaves (melissa officinalis l.), balm leaves, extract (melissa officinalis l.), balm, oil (melissa officinalis l.) fir, balsam, needles and twigs (abies balsamea (l.) mill.), balsam fir, oil (abies balsamea (l.) mill.), balsam fir, oleoresin (abies balsamea (l.) mill.), balsam, peru (myroxylon pereirae klotzsch), balsam, peru, oil (myroxylon pereirae klotzsch), basil (ocimum basificum l.), basil bush (ocimum minimum l.), basil, extract (ocimum basilicum l.), basil, oil (ocimum basilicum l.), basil, oleoresin (ocimum basilicum l.), bay (laurus nobilis l.), bay leaves, sweet, extract (laurus nobilis l.), bay leaves, sweet, oil (laurus nobilis l.), bay leaves, west indian, extract (pimenta acris kostel), bay leaves, west indian, oil (pimenta racemosa (mill.) j. w. moore), bay leaves, west indian, oleoresin (pimenta acris kostel), beechwood, creosote (fagus spp.), benzoin, resin (styrax spp.), bergamot, oil (citrus aurantium l. subsp. bergamia Wright et arn.), birch, sweet, oil (betula lenta l.), birch tar, oil (betula pendula roth and related betula spp.), blackberry bark, extract (rubus, spp. of section eubatus), blackberry fruit extract, bois de rose, oil (aniba rosaeodora ducke), boldus leaves (peumus boldus mol.), bonito, dried, boronia, absolute (boronia megastigma nees), bouillon, vegetable, smoke, bryonia root (bryonia spp.), buchu leaves (barosma betulina and crenulata), buchu leaves extract, buchu leaves, oil (barosma spp.), buckbean leaves (menyanthes trifoliata l.), buckbean leaves, extract (menyanthes trifoliata l.), cajeput, oil (melaleuca leucadendron l.), calumba root (jatrorrhiza palinata (lam.) calumba root, extract (jatrorrhiza palmata (lam) miers), d-camphor, camphor, japanese, white, oil (cinnamomum camphora (l.) nees et eberm.), camphor oil, formosan ho-sho, leaves (cinnamomum camphora), cananga, oil (cananga odorata hook. f and thoms.), candelilla wax (wax from stems and branches of euphorbia cerifera), capers (capparis spinosa l.), capsicum (capsicum spp.), capsicum extract (capsicum spp.), capsicum, oleoresin (capsicum spp.), caramel, caraway (carum carvi l.), caraway, black (nigella sativa l.), caraway, oil (carum carvi l.), cardamom (elletaria cardamomum (l.) maton), cardamom oleoresin, cardamom seed, oil (elletaria cardamomum (l.) maton), carob bean, extract (ceratonia siliqua l.), cascara, bitterless, extract (rhamnus purshiana dc.), cascarilla bark, extract (croton spp.), cascarilla bark, oil (croton spp.), casein, cassia buds (cinnamomum cassia blume), cassie, absolute (acacia farnesiana (l.) willd.), castoreum, extract (castor spp.), castoreum, liquid (castor spp.), castor oil (ricinus communis l.), catechu, black, extract (acacia catechu willd.), catechu, black, powder (acacia catechu willd.), cedar leaf, oil (thuja occidentalis l.), cedarwood oil alcohols, cedarwood oil terpenes, celery seed (apium graveolens l.), celery seed, extract (apium graveolens l.), celery seed, extract solid (apium graveolens l.), celery seed, oil (apium graveolens l.), celery seed, centaury (centaurium umbellatum gilib.), cereal solids, hydrolyzed, chamomile flower (matricaria chamomilla l.), chamomile flower (anthemis nobilis l.), chamomile flower, hungarian, oil (matricaria chamomilla l.), chamomile flower, oil (anthemis nobilis l.), chamomile flower, roman, extract (anthemis nobilis l.), char smoke flavor, cherry bark, wild, extract (prunus serotina ehrh.), cherry-laurel leaves (prunus laurocerasus l.), cherry laurel, oil (prunus laurocerasus l.) (ffpa), cherry-laurel water (prunus laurocerasus l.), cherry pits, extract (prunus spp.), chervil (anthriscus cerefolium (l.) hoffm,), chervil, extract (anthriscus cerefolium l.), chestnut leaves (castanea dentata (marsh.) borkh.), chestnut leaves, extract (castanea dentata (marsh) borkh.), chestnut leaves, extract solid (castanea dentata (marsh.) borkh.), chicory, extract (cichorium intybus l.), chilte (cnidoscolus (also known as jatropha) spp.), chiquibul (manilkara zapotilla gilly), chirata (swertia chirata buch.-ham.), chirata, extract (swertia chirata buch.-ham.), chives (allium schoenoprasum l.), cinchona bark, red (cinchona succirubra pav. or its hybrids), cinchona bark, red, extract (cinchona succirubra pav. or its hybrids), cinchona bark, yellow (cinchona spp.), cinchona bark, yellow, extract (cinchona spp.), cinchona, extract (cinchona spp.), cinnamon (cinnamomum spp.), cinnamon bark, extract (cinnamomum spp.), cinnamon bark, oil (cinnamomum spp.), cinnamon bark oleoresin, ceylon, chinese, or Saigon (cinnamomum spp.), cinnamon leaf, oil (cinnamomum spp.), cinnamon leaf oil, rectified, citronella, oil (cymbopogon nardus rendle), citrus peels, extract (citrus spp.), clary (salvia sclarea l.), clary sage, absolute, clary, oil (salvia sclarea l.), clary sage, clove bud, extract (eugenia spp.), clove bud, oil (eugenia spp.), clove bud, oleoresin (eugenia spp.), clove leaf, oil (eugenia spp.), clover (trifolium spp.), clover, extract (trifolium spp.), clover herb distillate, clover, oil (trifolium spp.), clover tops, red, extract solid (trifolium pratense l.), cloves (eugenia spp.), clove stem, oil (eugenia spp.), coca leaf, extract (decocainized) (erythroxylon coca lam.), cocoa extract, cocoa with dioctyl sodium sulfosuccinate, refined, coffee concentrate, pure, coffee extract (coffea spp.), coffee extract, solid, cognac, green, oil, cognac, white, oil, copaiba (south american spp. of copaifera l.), copaiba, oil (south american spp. of copaifera ), coriander (coriandrum sativum l.), coriander leaf oil (coriandrum sativum l.), coriander, oil (coriandrum sativum l.), cork, oak (quercus spp.), corn silk extract (zea mays l.), corn silk, oil (zea mays l.), costmary (chrysanthemum balsamita l.), costus root, oil (saussurea lappa clarke), cottonseed flour, defatted, cottonseed flour, partially defatted, cooked, cottonseed flour, partially defatted, cooked, toasted, cottonseed kernels, glandless, raw, cottonseed kernels, glandless, roasted, cubeb (piper cubeba l. f.), cubeb, oil (piper cubeba l. f.), cubebol, cumin (cuminum cyminum l.), cumin, oil (cuminum cyminum l.), currant buds, black, absolute (ribes nigrum l.), currant juice, black, currant leaves, black (ribes nigrum l.), daidai peel oil, damiana leaves (turnera diffusa willd.), dandelion, fluid extract (taraxacum spp.), dandelion root, extract solid (taraxacum spp.), davana oil (artemesia pallens wall.), dehydrated beets, dill (anethum graveolens l.), dill, oil (anethum graveolens l.), dill seed, indian (anethum spp.), dill seed oil (anethum sowa roxb.), dittany of crete (origanum dictamnus l.), dittany (fraxinella) roots (dictamnus albus l.), dog grass, extract (agropyron repens (l.) beauv.), dragon's blood, extract (daemonorops spp. or other botanical sources), dried algae meal, elder flowers (sambucus canadensis l. or sambucus nigra l.), elder flowers, extract (sambucus canadensis l. or sambucus nigra l.), elder tree leaves (sambucus nigra l.), elecampane root, extract (inula helenium l.), elecampane root, oil (inula helenium l.), elemi, gum, elemi, oil (canarium spp.), erigeron, oil (erigeron canadensis l.), eucalyptol, eucalyptus, oil (eucalyptus globulus labille), eugenol, fennel, common (foeniculum vulgare mill.) fennel, sweet (foeniculum vulgare mill. var. dulce (d.c.) alef.), fennel, sweet, oil (foeniculum vulgare mill. var. duke (d.c.) alef.), fenugreek (trigonella foenum-graecum l.), fenugreek, extract (trigonella foenum-graecum l.), fenugreek, oleoresin (trigonella foenum-graecum l.), fir (pine) needles and twigs (abies sibirica ledeb.), fir needles and twigs, oil (abies spp.), fish oil (hydrogenated), fish protein concentrate, whole, fish protein isolate, fruit juice, galanga, greater (alpinia galanga willd), galangal root (alpinia spp.), galangal root, extract (alpinia spp.), galangal root, oil (alpinia spp.), galbanum, oil (ferula spp.), galbanum, resin (ferula spp.), gambir (uncaria gambir roxb.), gardenia gummifera distillate, garlic, garlic extract, garlic, oil (allium sativum l.), gelatin, genet, absolute (spartium junceum l.), genet, extract (spartium junceum l.), gentian root, extract (gentiana lutea l.), gentian, stemless (gentiana acaulis l.), geranium (pelargonium spp.), geranium, east indian, extract (cymbopogon martini stapf.), geranium, east indian, oil (cymbopogon martini stapf.), geranium extract (pelargonium spp.), geranium, oil (pelargonium spp.), geranium, rose, oil (pelargonium graveolens l'her.), germander, chamaedrys (teucrium chamaedrys l.), germander, chamaedrys, extract (teucrium chamaedrys l.), germander, chamaedrys, extract solid (teucrium chamaedrys l.), germander, golden (teucrium polium l.), ghatti, gum (anogeissus latifolia wall.), ginger (zingiber officinale rosc.), ginger, extract (zingiber officinale rosc.), ginger, oil (zingiber officinale rosc.), ginger, oleoresin (zingiber officinale rosc.), glycyrrhizin, ammoniated (glycyrrhiza spp.), grains of paradise (aframomum melegueta (rosc.) k. schum.), grape color extract, grape essence, natural, grapefruit essence, natural, grapefruit, extract, grapefruit, juice, grapefruit, oil (citrus paradisi macf.), grapefruit oil, conc., grapefruit, oil, terpeneless (citrus paradisi), grape seed extract, grape skin extract, ground limestone, guaiac gum (guaiacum spp.), guaiac gum, extract (guaiacum spp.), guaiac wood, extract (guaiacum spp.), guaiac wood, oil (guaiacum spp.), guarana, gum (paullinia cupana hbk), guarana seed, extract, guava (psidium spp.), guava extract, gum arabic, gutta hang kang (palaquium leiocarpum boerl. and p. oblongifolium burck.), haematococcus algae meal, haw bark, black, extract (viburnum prunifolium l.), heliopsis longipes extract, hemlock (tsuga spp.), hemlock needles and twigs, oil (tsuga spp.), hickory bark, extract (carya spp.), hickory smoke dist., honeysuckle extract, hops, extract (humulus lupulus l.), hops extract, modified, hops, extract solid (humulus lupulus l.), hops, oil (humulus lupulus l.), horehound extract (maruubium vulgare l.), horehound (marrubium vulgare l.), horehound solid, extract, horsemint leaves, extract (monarda spp.), horseradish (armoracia lapathifolia gilib.), horseradish oil, hyacinth, absolute (hyacinthus orientalis l.), hyacinth flowers (hyacinthus orientalis l.), hyssop, extract (hyssopus officinalis l.), hyssop (hyssopus officinalis l.), hyssop, oil (hyssopus officinalis l.), iceland moss (cetraria islandica ach.), immortelle, absolute (helichrysum angustifolium dc), immortelle, extract (helichrysum angustifolium dc.), imperatoria (peucedanum ostruthium (l.) koch (imperatoria ostruthium l.)), iva (achillea moschata jacq.), iva, extract (achillea moschata jacq.), jambu oleoresin, japan wax, jasmine, absolute (jasminum spp.), jasmine, concrete (jasminum spp.), jasmine, oil (jasminum grandiflorum l.), jasmine, spiritus (jasminum grandiflorum l.), jelutong (dyera costulata hook, f. and d. lowii hook, f.), juniper (berries) (juniperus communis l.), juniper, extract (juniperus communis l.), juniper oil (juniperus communis l.), karaya, gum (sterculia urens roxb.), kelp, kola nut, extract (cola acuminata schott et endl.), labdanum, absolute (cistus spp.), labdanum, oil (cistus spp.), labdanum, oleoresin (cistus spp.), laurel berries (laurus nobilis l.), lavandin absolute, lavandin, concrete, lavandin, oil, lavender, absolute (lavandula officinalis chaix), lavender, concrete (lavandula officinalis chaix), lavender (lavandula officinalis chaix), lavender, oil (lavandula officinalis chaix), lavender, spike (lavandula latifolia bill.), lavender, spike, oil (lavandula spp.), leche carpi (couma macrocarpa barb. rodr.), leche de vaca (brosimum utile (h.b.k.) pittier, and poulsenia spp.), leek oil, lemon essence, lemon, extract (citrus limon (l) burm. f), lemon grass, oil (cymbopogon citratus dc. and cymbopogon flexuosusstapf), lemon, juice, lemon, oil (citrus limon (l.) burm. f.), lemon, oil, terpeneless (citrus limon burm. f.), lemon peel extract, lemon peel granules, lemon terpenes, lemon-verbena (lippia citriodora hbk.), lemon verbena, oil (lippia citriodora), licorice extract (glycyrrhiza spp.), licorice extract powder (glycyrrhiza spp.), licorice (glycyrrhiza spp.), lime, essence, lime, juice, lime juice, dehydrated, lime oil, distilled, lime oil, expressed, lime, oil, terpeneless (citrus aurantifolia (christman) swingle), linaloe wood, oil (bursera delpechiana poiss. and other bursera spp.), linden flowers, extract (tilia spp.), linden flowers (tilia glabra vent.), linden leaves (tillia spp.), litsea cubeba berry oil, locust (carob) bean gum, toyage, extract (levisticum officinale koch), lovage (levisticum officinale koch), lovage, oil (levisticum officinale koch), luo han fruit concentrate, lupulin (humulus lupulus l.), mace (myristica fragrans houtt.), mace, oil (myristica fragrans houtt.), mace, oleoresin (myristica fragrans houtt.), maidenhair fern (adiantum capillus-veneris l.), malt syrup (malt extract), mandarin, oil (citrus reticulata blanco), marigold, pot (calendula officinalis l.), marjoram, oleoresin (marjorana hortensis moench (origanum majorana l.)), marjoram, pot (majorana onites (l.) benth. (origanum vulgare l.)), marjoram seed (majorana hortensis moench (origanum majorana l.)), marjoram, sweet (majorana hortensis moench (origanum majorana l.)), marjoram, sweet, oil (majorana hortensis moench (origanum majorana l.)), massaranduba balata (manilkara huberi (ducke) chevalier), massaranduba balata, solvent-free resin extract, massaranduba chocolate (manilkara solimoesensis gilly), massoia bark oil, mastic gum, mate, absolute (ilex paraguariensis st. hil.), mate, leaves, menhaden oil, menhaden oil, hydrogenated, menhaden oil, partially hydrogenated, menthol, mesquite wood extract, milk powder, whole, enzyme-modified, mimosa, absolute (acacia decurrens willd. var. dealbata), mimosa concrete (acacia decurrens willd. var. dealbata), mineral oil, white, molasses, concentrate, molasses, extract (saccharum officinarum l.), molasses (saccharum officinarum l.), mountain maple (acer spicatum lam.), mountain maple bark (acer spicatum lam.), mountain maple, extract solid (acer spicatum lam.), mullein flowers (verbascum spp.), mushroom oil, distilled, musk ambrette, musk, ketone, musk tonquin (moschus moschiferus l.), mustard, brown (brassica spp.), mustard, brown, extract (brassica spp.), mustard flour, mustard oil, mustard, oriental, mustard, yellow (brassica spp.), mustard, yellow, extract (brassica spp.), myrrh, extract, myrrh, gum (commiphora spp.), myrrh, oil (commiphora spp.), myrtle leaves (myrtus communis l.), myrtle, oil (myrtus communis l.), naringin, extract (citrus paradisi macf.), nisin preparation, nutmeg (myristica fragrans houtt.), nutmeg, oil (myristica fragrans houtt.), white, extract (quercus alba l.), oak moss, absolute (evernia spp.), oak moss, concrete (evernia prunasti spp.), oak wood, english (quercus robur l.), oat gum, oiticica oil, oleic acid, oleic acid, from tall oil fatty acids, olestra, oleyl alcohol, olibanum, absolute (boswellia spp.), olibanum, gum, resin (boswellia spp.), olibanum, oil (boswellia spp.), olibanum, resinoid (boswellia spp.), onion, oil (allium cepa l.), opopanax, gum, opopanax, non-specific, opopanax, oil, opopanax tincture, orange b, orange essence, natural, orange essence oil, natural, orange, extract, orange flowers, absolute (citrus aurantium l.), orange flowers, bitter (citrus aurantium l.), orange, juice, orange leaf, absolute (citrus aurantium l.), orange, oil, distilled (citrus sinensis (l.) osbeck), orange, oil, terpeneless (citrus sinensis (l.) osbeck), orange peel, orange peel, bitter, extract (citrus aurantium l.), orange peel, bitter, oil (citrus aurantium l.), orange peel, sweet, extract (citrus sinensis (l.) osbeck), orange peel, sweet, oil (citrus sinensis (l.) osbeck), orange peel, sweet, oil, terpeneless (citrus sinensis (l.) osbeck), oregano, european (origanum spp.), oregano (lippia spp., usually l. graveolens hbk), oregano (other genera including coleus, lantana and hyptis), origanum oil (extractive) (thymus capitates hoff. et link), orin lactone, 1-ornithine monochlorohydrate/ornithine, orris, concrete, liquid, oil (iris florentina l.), orris root, extract (iris florentina l.), osmanthus absolute, ox bile extract, pansy (viola tricolor l.), papain (carica papaya l.), paprika (capsicum annuum l.), paprika oleoresin (capsicum annuum l.), parsley, oil (petroselinum spp.), parsley, oleoresin (petroselinum spp.), parsley (petroselinum spp.), passion flower extract, passion flower (passiflora incarnata l.), patchouly, oil (pogostemon spp.), peach kernel, extract (prunus persica sieb et zucc.), peach leaves, extract (prunus persica (l.) batsch), peach leaves (prunus persica (l.) batsch), peanut oil, peanut stearine (arachis hypogaea l.), pecan shell flour, pennyroyal, oil, american (hedeoma pulegiodes (l.)), pennyroyal, oil, european (mentha pulegium l.), pepper, black, oil (piper nigrum l.) pepper, black, oleoresin (piper nigrum l.), pepper, black (piper nigrum l.), pepper, cayenne, peppermint leaves (mentha piperita l.), peppermint, oil (mentha piperita l.), peppermint plant, pepper, red, pepper, white, oil (piper nigrum l.), pepper, white, oleoresin (piper nigrum l.), pepper, white (piper nigrum l.), perilla leaf oil, petitgrain, lemon, oil (citrus limon (l.) burm. f.), petitgrain, mandarin, oil (citrus reticulata blanco var. mandarin), petitgrain, oil (citrus aurantium l.), pimenta leaf, oil (pimenta officinalis lindl.), 3-pinanone, pine bark, white, extract solid (pinus strobus l.), pine bark, white, oil (pinus strobus l.), pine bark, white (pinus strobus l.), pine needle, dwarf, oil (pinus mugo turra var. pumilio (haenke) zenari), pine, scotch, oil (pinus sylvestris l.), pine tar, oil (pinus spp.), pine, white, oil (pinus spp.), pomegranate bark, extract (punica granatum l.), poplar buds (populus spp.), poppy seed (papaver somniferum l.), prickly ash bark extract (xanthoxylum spp.), prickly ash bark, oil, protein, animal, hydrolyzed, protein hydrolysate, unspecified, protein, milk, hydrolyzed, protein, vegetable, hydrolyzed, pulegone, extract (picrasma exceisa (sw.) planch or quassia amara l.), quebracho bark extract, quillaia extract (quillaja saponaria molina), quillaia (quillaja saponaria molina), quince seed, extract (cydonia spp.), rhatany, extract (krameria spp.), rhubarb, garden root (rheum rhaponticum l.), rhubarb root (rheum spp.), rose, absolute (rosa spp.), rose, bud (rosa spp.), rose flowers (rosa spp.), rose hips, extract (rosa spp.), rose leaves (rosa spp.), roselle (hibiscus sabdariffa l.), rosemary, extract (rosmarinus officinalis l.), rosemary, oil (rosemarinus officinalis l.), rosemary, oleoresin, rosemary (rosemarinus officinalis l.), rose, oil (rosa spp.), rose water, stronger (rosa centifolia l.), rosidinha (micropholis (also known as sideroxylon) spp.), rosin, glycerol ester, rosin, gum, glycerol ester, rosin, gum or wood, partially hydrogenated, glycerol ester, rosin, gum or wood, partially hydrogenated, pentaerythritol ester, rosin, gum or wood, pentaerythritol ester, rosin, limed, rosin, methyl ester, partially hydrogenated, rosin, partially dimerized, calcium salt, rosin, partially dimerized, glycerol ester, rosin, partially hydrogenated, rosin (pinus spp.) and rosin derivatives, rosin, polymerized, glycerol ester, rosin, tall oil, glycerol ester, rosin, wood, rosin, wood, glycerol ester, rosin, wood, oil (ruta graveolens l.), rue (ruta graveolens l.), rum, saffron (crocus sativus l.), saffron, extract (crocus sativus l.), safrole-free extract of sassafras, safrole-prohibited, sage, greek (salvia triloba l.), sage, oil (salvia officinalis l.), sage, oleoresin (salvia officinalis l.), sage (salvia officinalis l.), sage, spanish, oil (salvia lavandulaefolia vahl.), sandalwood, red (pterocarpus santalinus Lf.), sandalwood, white (santalum album l.), sandalwood, yellow, oil (santalum album l.), sandarac (tetraclinis articulate (vahl.) mast.), sarcodactylis oil, sarsaparilla, extract (smilax spp.), sassafras bark, extract (safrole-free) (sassafras albidum (nutt.) nees), sassafras leaves (safrole-free) (sassafras albidum (nutt.) nees), sausage casing (hcl and cellulose fibers), savory, summer, oil (satureja hortensis l.), savory, summer, oleoresin (satureja hortensis l.), savory, summer (satureja hortensis l.), savory, winter, (satureja montana l.), savory, winter, oleoresin (satureja montana l.), savory, winter (satureja l.) montana schinus molle, oil (schinus molle l.), scotch spearmint oil, senna, alexandria (cassia acutifolia delile), serpentaria (aristolochia serpentaria l.), sesame (sesamum indicum l.), silver fir, needles and twigs, oil (abies alba mill.), silver-silver dragees, simaruba bark (simaruba amara aubl.), sloe berries, extract (prunus spinosa l.), sloe berries, extract solid (prunus spinosa l.), sloe berries (prunus spinosa l.), snakeroot, canadian, oil (asarurn canadense l.), soya bean oil fatty acids, hydroxylated, soya fatty acid amine, ethoxylated, soybean oil, epoxidized, soybean oil, hydrogenated, soy protein concentrate, enzyme activated, soy protein, isolate, spearmint, extract (mentha spicata l.), spearmint (mentha spicata l.), spearmint, oil (mentha spicata l.), spikenard extract, spruce needles and twigs, extract (picea spp.), spruce needles and twigs, oil (picea spp.), st. johnswort leaves, flowers and caulis (hypericum perforatum l.), storax extract (liquidambar spp.), storax (liquidambar spp.), storax oil, sugar beet juice extract, sugar beet extract flavor base, sweet blackberry leaves extract, tagetes meal & extract, tagetes, oil (tagetes spp.), tallow alcohol, hydrogenated, tallow, beef, tallow flakes, tallow, hydrogenated, tallow, hydrogenated, oxidized or sulfated, tamarind extract (tamarindus indica l.), tamarinds, tangerine, essence, tangerine, extract (citrus reticulata blanco), tangerine, oil (citrus reticulata blanco), tannic acid, tansy, oil (tanacetum vulgara l.), tansy (tanacetum vulgara l.), tarragon (artemisia dracunculus l.), tarragon extract (artemisia dracunculus l.), tarragon oil (artemisia dracunculus l.), tea extract (thea sinensis l.), tea tree oil (melaleuca alternifolia), blessed (cnicus benedictus l.), thistle, blessed, extract (cnicus benedictus l.), thistle, blessed, extract solid (cnicus benedictus l.), thistle, blessed, oil (cnicus benedictus l.), thyme, extract, thyme oil (thymus vulgaris l. and t. zygis var. gracilis boiss.), thyme oleoresin, thyme (thymus serpyllum l.), thyme (thymus vulgaris l.), thyme, wild or creeping, extract (thymus serpyllum l.) thymol, balsam, extract (myroxylon spp.), tolu, balsam, gum (myroxylon spp.), tragacanth, gum (astragalus spp.), trefoil, sweet (melilotus coerulea), triacetin (glycerol triacetate), oil (polianthes tuberosa l.), tunu (castilla fallax cook), turmeric (curcuma longa l.), turmeric, extract (curcuma longa l.), turmeric, oleoresin (curcuma longa l.), valerian root, extract (valeriana officinalis l.), valerian root, oil (valeriana officinalis l.), vanilla, absolute (vanilla spp.), vanilla, extract (vanilla spp.), vanilla, oleoresin (vanilla spp.), vanilla (vanilla spp.), vegetable juice, veronica (veronica officinalis l.), vervain, european (verbena officinalis l.), vetiver, oil (vetiveria zizanioides stapf), vetiverol, vetiver (vetiveria zizaniodes stapf), violet leaves absolute (viola odorata l.), violet, swiss (viola calcarata l.), walnut hull, extract (juglans spp.), walnut leaves, extract (juglans spp.), wheat gluten, whey, whey, delactosed, whey, demineralized, whey, partially dimineralized and partially delactosed, whey protein concentrate, wintergreen, extract (gaultheria procumbens l.), wintergreen, oil (gaultheria procumbens l.), woodruff, sweet (asperula odorata l.), wort, yarrow, herb (achillea millefolium l.), yarrow, oil (achillea millefolium l.), yeast autolysate, yeast, dried irradiated, yeast extract autolyzed, yeast-malt sprout extract, yeasts, yeasts, dried, yerba santa, fluid extract (eriodictyon californicum (hook and arn) torr), ylang-ylang, oil (cananga odorata hook. f. and thomas), yucca, joshua-tree (yucca brevifolia engelm.), yucca, mohave, extract (yucca spp.), and acai berry extract

In some preferred embodiments, the composition comprises an umami flavor compound or umami flavor enhancing compound and one or more plant or animal products where the plant or animal product is a culinary herb or spice. Representative culinary herbs and spices include but are not limited to: carrot, dehydrated carrot, carrot extract, parsnip, dehydrated parsnip, parsnip extract, onion, dehydrated onion, onion powder, onion flakes, onion extract, garlic, dehydrated garlic, garlic flakes, garlic powder, garlic extract, buttermilk, buttermilk powder, buttermilk solids, whey, whey powder, whey solids, milk, reduced fat milk, milk powder, milk solids, cream, cheese, cheese powder, cheese solids, cheese cultures, annatto, turmeric, sodium caseinate, milk protein, enzymatic digest of milk protein, tomato, tomato paste, tomato powder, tomato sauce, tomato extract, dehydrated tomato, sour cream, sour cream solids, caramel color, soy flour, chives, dehydrated chives, chive powder, ajwain, carom seeds (trachyspermum ammi), akudjura (solanum centrale), alkanet (alkanna tinctoria), for red color, alligator pepper, mbongo spice (mbongochobi), hepper pepper (aframomum danielli, a. citratum, a. exscapum), allspice (pimenta dioica), angelica (angelica archangelica), anise (pimpinella anisum), anise hyssop (agastache foeniculum), aniseed myrtle (syzygium anisatum), annatto (bixa orellana), apple mint (mentha suaveolens, mentha×rotundifolia and mentha×villosa), artemisia (artemisia spp.), asafbetida (ferula assafoetida), asarabacca (asarum europaeum), avens (geum urbanum), avocado leaf (persea americana), barberry (berberis vulgaris and other berberis spp.), basil, sweet (ocimum basilicum), basil, holy (ocimum tenuiflorum), basil, lemon (ocimum×citriodorum), basil, thai (o. basilicum var. thyrsiflora), bay leaf (laurus nobilis), bay leaf, iridian, tejpat, malabathrum, bee balm (monarda didyma), boldo (peumus boldus), borage (borago officinalis), blue fenugreek, blue metilot (trigonella caerulea), caper (capparis spinosa), caraway (carum carvi), cardamom (elettaria cardamomum), cardamom, black (amomum subulatum, amomum costatum), cassia (cinnamomum aromaticum), catnip (nepeta cataria), cayenne pepper (capsicum annuum), celery leaf (apiumi graveolens), celery seed (apiumi graveolens), chervil (anthriscus cerefolium), chicory (cichorium intybus), chili pepper (capsicum spp.), cicely, sweet cicely (myrrhis odorata), cilantro, coriander greens, coriander herb (coriandrum sativum), cinnamon, indonesian (cinnamomum burmannii, cassia vera), cinnamon, saigon or vietnamese (cinnamomum loureiroi), cinnamon, true or ceylon (cinnamomum verum, c. zeylanicum), cinnamon, white (canella winterana), cinnamon myrtle (backhousia myrtifolia), clary, clary sage (salvia sclarea), clove (syzygium aromaticum), coriander seed (coriandrum sativum), coriander, vietnamese (persicaria odorata), costmary (tanacetum balsamita), cubeb pepper (piper cubeba), cudweed (gnaphalium spp.), culantro, culangot, long coriander (eryngium foetidum), cumin (cuminum cyminum), curry leaf (murraya koenigii), curry plant (helichrysum italicum), dill herb or weed (anethum graveolens), dill seed (anethum graveolens), elderflower (sambucus spp.), epazote (dysphania ambrosioides), fennel (foeniculum vulgare), fenugreek (trigonella foenum-graecum), file powder, gumbo filé (sassafras albidum), fingerroot, krachai, tem kuntji (boesenbergia rotunda), galangal, greater (alpinia galanga), galangal, lesser (alpinia officinarum), gatingale (cyperus spp.), garlic chives (allium tuberosum), ginger (zingiber officinale), ginger, torch, bunga siantan (etlingera elatior), golpar, persian hogweed (heracleum persicum), grains of paradise (aframomum melegueta), grains of selim, kani pepper (xylopia aethiopica), horseradish (armoracia rusticana), houttuynia cordata (vietnam), huacatay, mexican marigold, mint marigold (tagetes minuta), hyssop (hyssopus officinalis), indonesian bay leaf, daun salam (syzygium polyanthum), jasmine flowers (jasminum spp.), jiaogulan (gynostemma pentaphyllum), jimbu (allium hypsistum), juniper berry (juniperus communis), kaffir lime leaves, makrud lime leaves (citrus hystrix), kala zeera (or kala jira), black cumin (bunium persicum), kawakawa seeds (macropiper excelsum), ketuak, kluwak, kepayang (pangium edule), kencur, galangal, kentjur (kaempferia galanga), kink gioi, vietnamese balm (elsholtzia ciliata), kokam seed (garcinia indica), korarima, ethiopian cardamom, false cardamom (aframomum corrorima), koseret leaves (lippia adoensis), lavender (lavandula spp.), lemon balm (melissa officinalis), lemon ironbark (eucalyptus staigeriana), lemon myrtle (backhousia citriodora), lemon verbena (lippia citriodora), lemongrass (cymbopogon citratus, c, flexuosus, and other cymbopogon spp.), leptotes bicolor, lesser calamint (calamintha nepeta), niptella, nepitella , licorice, liquorice (glycyrrhiza glabra), lime flower, linden flower (tilia spp.), lovage (levisticum officinale), mace (myristica fragans), mahleb, st. lucie cherry (prunes mahaleb), marjoram (origanum majorana), mastic (pistacia lentiscus), mint (mentha spp.), 25 species, hundreds of varieties, mountain horopito (pseudowintera colorata), ‘pepper-plant’, musk mallow, abelmosk (abelmoschus moschatus), mustard, black, mustard plant, mustard seed (brassica nigra), mustard, brown, mustard plant, mustard seed (brassica juncea), mustard, white, mustard plant, mustard seed (sinapis alba), mustard, yellow (brassica hirta=sinapis alba), nigella, kalonji, black caraway, black onion seed (nigella sativa), njangsa, djansang (ricinodendron heudelotii), nutmeg (myristica fragrans), olida (eucalyptus olida), oregano (origanum vulgare, o. heracleoticum, and other species), oregano, cuban (plectranthus amboinicus), orris root (iris germanica, i. florentina, i. pallida), pandan flower, kewra (pandanus odoratissimus), pandan leaf, screwpine (pandanus amaryllifolius), paprika (capsicum annuum), paracress (spilanthes acmella, soleracea), parsley (petroselinum crispum), pepper, black, white, and green (piper nigrum), pepper, brazilian, or pink pepper (schinus terebinthifolius), pepper, dorrigo (tasmannia stipitata), pepper, long (piper longum), pepper, mountain, cornish pepper leaf (tasmannia lanceotata), peppermint (mentha piperata), peppermint gum leaf (eucalyptus dives), perilla, shiso (perilla spp.), peruvian pepper (schinus molle), quassia (quassia amara), rice paddy herb (limnophila aromatica), rosemary (rosmarinus officinalis), rue (ruta graveolens), safflower (carthamus tinctorius), saffron (crocus sativus), trade and use of saffron, sage (salvia officinalis), Saigon cinnamon (cinnamomum loureiroi), salad burnet (sanguisorba minor), salep (orchis mascula), sassafras (sassafras albidum), savory, summer (satureja hortensis), savory, winter (satureja montana), shiso (perilla frutescens), silphium, silphion, laser, laserpicium, lasarpicium , sorrel (rumex acetosa), sorrel, sheep (rumex acetosella), spearmint (mentha spicata), spikenard (nardostachys grandiflora or n. jatamansi), star anise (illicium verum), sumac (rhos coriaria), sweet woodruff (galium odoratum), szechuan pepper, sichuan pepper (zanthoxylum piperitum), tarragon (artemisia dracunculus), thyme (thymus vulgaris), thyme, lemon (thymus×citriodorus), turmeric (curcuma longa), vanilla (vanilla planifolia), voatsiperifery (piper borbonense), wasabi (wasabia japonica), water-pepper, smartweed (polygonum hydropiper), watercress (rorippa nasturtium-aquatica), waffleseed, wild thyme (thymus serpyllum), willow herb (epilobium parviflorum), wintergreen (gaultheria procumbens), wood avens, herb bennet (geum urbanum), woodruff (galium odoratum), wormwood, absinthe (artemisia absinthium), yerba buena, yarrow (achillea millefolium), za'atar (herbs from the genera origanum, calamintha, thymus, and satureja), zedoary (curcuma zedoaria), herb mixes, curly, tandoor masala, garam masala, chaat masala, tikka masala, masala dosa, paan masala, biryani masala, berbere, jerk seasonings, and spice rubs.

In some embodiments, the combination of the umami flavor compound or umami flavor enhancing compounds with the plant or animal products described above has a synergistic effect. For example, the combination may further enhance the umami flavor of the composition or enhance the properties of the plant or animal product.

### Fats, Oils, and Emulsions

In some preferred embodiments, the composition comprises an umami flavor compound or umami flavor enhancing compound and one or more fats, oils, or emulsions. Representative fats, oils, and emulsions include but are not limited to: corn oil, peanut oil, soybean oil, palm oil, cottonseed oil, coconut oil, canola oil, rapeseed oil, olive oil, safflower oil, sunflower oil, sesame oil, almond oil, beech nut oil, brazil nut oil, cashew oil, linseed oil, flaxseed oil, hazelnut oil, mongongo nut oil, pecan oil, pine nut oil, pistachio oil, walnut oil, grapeseed oil, grapefruit seed oil, lemon oil, orange oil, mineral oil, petrolatum, cucumber oil, bitter gourd oil, bottle gourd oil, buffalo gourd oil, butternut squash oil, squash oil, pumpkinseed oil, watermelon seed oil, hemp oil, castor oil, butter, lard, margarine, cocoa butter, butterfat, whale oil, sperm oil, fish oil, cod liver oil, shark liver oil, krill oil, shrimp oil, codfish oil, salmon oil, herring oil, anchovy oil, smelt oil, candlefish oil, sprat oil, sardine oil, mackerel oil, tilefish oil, tuna (Thunnus) oil, swordfish oil, macadamia oil, acai oil, blackcurrant seed oil, borage seed oil, evening primrose oil, amaranth oil, wheat oil, wheat germ oil, apricot oil, argan oil, artichoke oil, avocado oil, babassu oil, ben oil, tallow nut oil, borneo tallow nut oil, carob pod oil, carob oil, coriander seed oil, false flax oil, kapok seed oil, lallemantia oil, meadowfoam seed oil, mustard oil, okra seed oil, perilla seed oil, pequi oil, prune kernel oil, quinoa oil, ramtil oil, rice bran oil, rice oil, barley oil, barley seed oil, tea oil, tea tree oil, thistle oil, jatropha oil, cork tree oil, apple seed oil, balanos oil, bladderpod oil, brucea javanica oil, burdock oil, candlenut oil, carrot seed oil, chaulmoogra oil, crambe oil, cuphea oil, jojoba oil, mango oil, palm kernel oil, mowrah butter, neem oil, orange oil, rosehip seed oil, sea buckthorn oil, shea butter, snowball seed oil, tall oil, tamanu oil, tonka bean oil, chicken fat, schmaltz, beef fat, lamb fat, animal fat, tallowate, tallow, beef tallow, bacon fat, ham fat, suet, milk fat, olestra, stearic acid, lauric acid, linoleic acid, palmitic acid, palmitoleic acid, myristic acid, goose fat, duck fat, drippings from roasting, mayonnaise, and oil-in water or water-in oil emulsions.

In some embodiments, the combination of the umami flavor compound or umami flavor enhancing compounds with the fats, oils, or emulsions described above has a synergistic effect. For example, the combination may further enhance the umami flavor of the composition or enhance the properties of the fat, oil, or emulsion.

### Food or Beverage Products

In some embodiments, a composition incorporating an umami flavor agent as described herein may be included in food or beverage products or formulations. Examples of food and beverage products or formulations include, but are not limited to any foodstuff or item intended for ingestion, including any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon-like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to Bloody Mary, beer, malt liquor, malt beverages, cider/perry, flavored alcoholic beverages (FABs), wine, whiskey, flavored whiskey, rum, flavored rum, and other spirits. The soft drinks include, but are not limited to carbonates, such as colas and non-cola carbonates; fruit juice, such as juice, nectars, juice drinks and fruit flavored drinks; bottled water, which includes sparkling water, spring water, purified/table water, and vitamin water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips, crisps, flavored chips, flavored crisps, extruded snacks, tortilla/corn chips, flavored corn/tortilla chips, popcorn, flavored popcorn, pretzels, flavored pretzels, nuts, flavored nuts, and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, prepared snack cakes, doughnuts, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible products that are sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; marinated or seasoned fresh meats, and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fishlseafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chilled Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for ingestible compositions, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, soy and soy-based drinks, soy milk-based drinks, nut milk and nut-based drinks, including almond and cashew-based drinks, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, fresh sausages, smoked sausages, bacon, ham, smoked meats, frankfurters, luncheon meats, cold cuts, prepared deli meats, smoked fish/seafood, marinated fresh meat, flavor-treated fresh meat, broth-injected fresh meat, dry bread mixes, stuffings, dressings, stuffing mixes, dressing mixes, meat analogues, meat substitutes, frozen meat analogues, frozen meat substitutes, tempeh, tofu, seitan, textured vegetable protein, prepared meat substitutes, soy burgers, soy franks, soy and/or grain-based hamburger patty substitutes, rice, rice mixes, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seathod products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof.

Some embodiments provide medicaments, supplements, nutraceuticals, functional food products (e.g., any fresh or processed food claimed to have a health-promoting and/or disease-preventing properties beyond the basic nutritional function of supplying nutrients), pharmaceutical product, over the counter (OTC) product, oral care product, cosmetic products such as sweetened lip balms, and other personal care products including a flavor modifying composition as described herein.

In general, over the counter (OTC) product and oral care product generally refer to product for household and/or personal use which may be sold without a prescription and/or without a visit to a medical professional. Examples of the OTC products include, but are not limited to Vitamins and dietary supplements; Topical analgesics and/or anesthetic; Cough, cold and allergy remedies; Antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anesthetic include any topical creams/ointments/gels used to alleviate superficial or deep-seated aches and pains, e.g., muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral care product include, but are not limited to mouth cleaning strips, toothpaste, toothbrushes, mouthwashes/dental rinses, denture care, mouth fresheners at-home teeth whiteners, dentifrices, and dental floss.

In some embodiments, a flavor modifying composition as described herein may be provided in a flavoring concentrate formulation, e.g., suitable for subsequent processing to produce a ready-to-use (i.e., ready-to-serve) product. By “a flavoring concentrate formulation,” it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal.

In some embodiments the compositions described herein are included in a pet food, animal treat, or animal feed. In other embodiments, the compositions are applied to a veterinary medicament. In other embodiments, the compositions are applied to an animal oral health care product.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

1. A composition comprising one or more of the umami flavor compounds from the group consisting of: 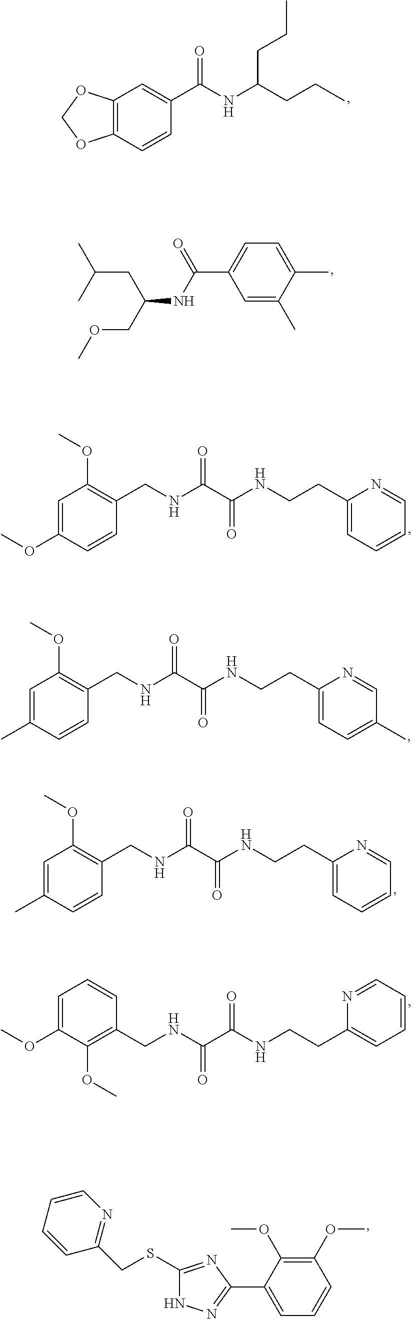 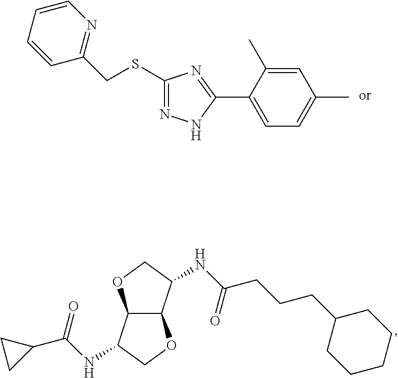 [SMILES: Cc1ccc(-c2nc(SCc3ccccn3)n[nH]2)c(C)c1 ; O=C(CCCC1CCCCC1)N[C@H]1CO[C@H]2[C@@H]1OC[C@@H]2NC(=O)C1CC1]
or salts thereof, and one or more food additives.

2. The composition according to claim 1 wherein the food additive is a flavorant or flavor enhancer.

3. The composition according to claim 2 wherein the food additive is a bitter flavorant, an acid or sour flavorant, a salt or salt flavor enhancer, a sweetener or sweet flavorant, or another umami flavorant or umami flavor enhancer.

4. (canceled)

5. (canceled)

6. (canceled)

7. (canceled)

8. The composition according to claim 2 wherein the food additive is an animal or plant product.

9. The composition according to claim 8 wherein the animal or plant product is an herb or spice.

10. The composition according to claim 2 wherein the food additive is a fat, oil, or emulsion.

11. The composition according to claim 1 comprising more than one food additive, such additives selected from the group consisting of bitter flavorants, acids, sour flavorants, salts, salt flavor enhancers, sweeteners, sweet flavorants, umami flavorants, umami flavor enhancers, animal products, plant products, herbs, spices, oils, emulsions, and non-flavorant food additives.

12. A method of modifying the flavor of a foodstuff, comprising incorporating the composition according to claim 1 into said foodstuff, wherein the composition comprises one, or optionally more than one food additive.

13. The foodstuff produced by the method of claim 12.

14. A method of modifying the flavor of a medicament, comprising incorporating the composition according to claim 1 into said medicament, wherein the composition comprises one, or optionally more than one food additive.

15. The medicament produced by the method of claim 14.

16. A method of modifying the flavor of an oral health product, comprising incorporating the composition according to claim 1 into said oral health product, wherein the composition comprises one, or optionally more than one food additive.

17. The oral health product produced by the method of claim 16.

18. A method of modifying the flavor of a pet food, animal feed, pet treat, veterinary medication, or animal health product, comprising incorporating the composition according to claim 1 into said pet food, animal feed, pet treat, veterinary medication, or animal health product, wherein the composition comprises one, or optionally more than one food additive.

19. The pet food, animal feed, pet treat, veterinary medication, or animal health product produced by the method of claim 18.

20. The foodstuff produced by the method of claim 12, wherein the composition comprises more than one food additive, such additives selected from the group consisting of bitter flavorants, acids, sour flavorants, salts, salt flavor enhancers, sweeteners, sweet flavorants, umami flavorants, umami flavor enhancers, animal products, plant products, herbs, spices, oils, emulsions, and non-flavorant food additives.

21. The medicament produced by the method of claim 14, wherein the composition comprises more than one food additive, such additives selected from the group consisting of bitter flavorants, acids, sour flavorants, salts, salt flavor enhancers, sweeteners, sweet flavorants, umami flavorants, umami flavor enhancers, animal products, plant products, herbs, spices, oils, emulsions, and non-flavorant food additives.

22. The oral health product produced by the method of claim 16, wherein the composition comprises more than one food additive, such additives selected from the group consisting of bitter flavorants, acids, sour flavorants, salts, salt flavor enhancers, sweeteners, sweet flavorants, umami flavorants, umami flavor enhancers, animal products, plant products, herbs, spices, oils, emulsions, and non-flavorant food additives.

23. The pet food, animal feed, pet treat, veterinary medication, or animal health product produced by the method of claim 18, wherein the composition comprises more than one food additive, such additives selected from the group consisting of bitter flavorants, acids, sour flavorants, salts, salt flavor enhancers, sweeteners, sweet flavorants, umami flavorants, utnami flavor enhancers, animal products, plant products, herbs, spices, oils, emulsions, and non-flavorant food additives.
